(19) United States
(12) Patent Application Publication
(10) Pub. No.: US 2016/0361270 A1
(43) Pub. Date: Dec. 15, 2016

(54) UNIFORM, HIGH BASIS WEIGHT NANOFIBER FABRICS FOR MEDICAL APPLICATIONS

(71) Applicant: University of Washington, Seattle, WA (US)

(72) Inventors: Ryan Stoddard, Seattle, WA (US); Richard Alan Edmark, Seattle, WA (US); Edward P. Roberts, Seattle, WA (US); Joseph-Tin Chan Phan, Seattle, WA (US); Kim Woodrow, Seattle, WA (US)

(73) Assignee: University of Washington, Seattle, WA (US)

(21) Appl. No.: 15/178,081

(22) Filed: Jun. 9, 2016

Related U.S. Application Data
(60) Provisional application No. 62/173,256, filed on Jun. 9, 2015.

(51) Int. Cl. A61K 9/70; A61K 31/683; A61K 31/46; A61K 31/505; A61K 31/513; D01D 5/00; A61K 31/567

(52) U.S. Cl. CPC A61K 9/70; D01D 5/0092; A61K 31/683; A61K 31/567; A61K 31/505; A61K 31/513; A61K 31/46; D10B 2509/00

(57) ABSTRACT

Described herein are devices, compositions and methods relating to the production of high basis weight, non-woven nanofiber polymer fabrics. In certain embodiments, described herein are modifications to free-surface, needle-less or nozzle-less electrospinning devices that permit the production of such high basis weight, non-woven nanofiber polymer fabrics. Also described are the fabrics themselves and the fabrics including one or more biologically active agents to be released upon contact with a biological tissue. Such fabrics can incorporate biologically active agents in various combinations that permit, for example, burst and/or sustained release kinetics of one or more, preferably two or more biologically active agents.

### CROSS-REFERENCE TO RELATED APPLICATIONS

This application claims benefit under 35 U.S.C. §119(e) to U.S. provisional patent application No. 62/173,256, filed Jun. 9, 2015, the contents of which are herein incorporated by reference in its entirety.

### GOVERNMENT SUPPORT

This invention was made with government support under Grant No. RO 1 AI112002 awarded by the National Institutes of Health (NIH). The government has certain rights in the invention.

### TECHNICAL FIELD

The technical field relates to nanofiber compositions and devices and methods for producing the same.

### BACKGROUND

Electrospun nanofibers have been broadly investigated for use as medical fabrics in applications of drug delivery, tissue engineering, and wound healing. Pharmaceutical applications of nanofibers require a scalable process and precise fabric homogeneity and drug loading, which have not previously been demonstrated on a manufacturing scale instrument. Free surface or “needleless” electrospinning is a versatile and scalable method being evaluated for high throughput nanofiber production. A recent development in manufacturing scale needleless electrospinning equipment is the oscillating carriage method for solution entrainment onto a stationary wire electrode. However, a narrow physical understanding of this method has constrained its applications exclusively to low basis weight nanofiber coatings in the filtration industry. In contrast to filtration coatings, electrospun medical fabrics are more challenging to manufacture due to requirements for fabricating high basis weight, stand-alone materials that are needed to realize certain clinical applications.

### SUMMARY

Provided herein, in some aspects, are nozzle-less electrospinning devices, such devices comprising:
an electrospinning electrode and a collecting electrode comprising connections for a DC power supply, the electrospinning electrode and the collecting electrode spaced apart and establishing an electric field between the electrospinning electrode and the collecting electrode when DC power is supplied;
the collecting electrode comprising a first end and a second end,
the electrospinning electrode comprising a continuously fed or static charged electrode member partially submerged in or carrying an entrained polymer solution to permit electrospinning of fibers of the polymer towards the collecting electrode;
a substrate located between the electrospinning electrode and the collecting electrode such that electrospun polymer fibers become deposited on the substrate when the device is in use;
a first insulating material member encircling the collecting electrode and extending along the collecting electrode from the first end of the collecting electrode towards the second end of the collecting electrode such that a portion of the electrode is covered by the insulating material member and a gap of exposed collecting electrode is formed extending from an end of the insulating material member towards the second end of the collecting electrode;
such that the first insulating material member increases the uniform area of an electrospun polymer mat deposited on the substrate relative to the uniform area of a polymer mat deposited in the absence of the insulating material member.

In some embodiments of these devices and all such devices described herein, the device further comprises a second insulating material element encircling the collecting electrode and extending along the collecting electrode from the second end of the collecting electrode toward the first end of the collecting electrode such that a portion of the collecting electrode is covered by the second insulating material member and the gap of exposed collecting electrode extends between the first and second insulating material elements.

In some embodiments of these devices and all such devices described herein, the first insulating material member has a dielectric constant of at least 1.2.

In some embodiments of these devices and all such devices described herein, the first insulating material is selected from rubber, glass, cotton, perlite, charcoal, wood, fiberglass, fiberglass insulation, polyethylene, high density polyethylene, low density polyethylene, polypropylene, polystyrene and foamed versions of polyethylene, polypropylene and polystyrene, polyvinyl alcohol and polytetra fluoroethylene. In some embodiments of these devices and all such devices described herein, the first insulating material is butyl rubber.

In some embodiments of these devices and all such devices described herein, the uniform area of the electrospun polymer mat deposited on the substrate by the device in the presence of the first insulating material member is at least 25 cm (e.g., in the “cross-direction” or “carriage direction” (CD) dimension) by 100 cm (e.g., in the “machine direction” (MD) dimension).

In some embodiments of these devices and all such devices described herein, the substrate is substantially planar.

In some embodiments of these devices and all such devices described herein, the substrate is selected from waxed paper, parchment paper, silicone coated paper, Quilon coated paper, glassine paper, polypropylene spunbond, cellulosic paper, aluminum foil, copper foil and polytetra fluoroethylene sheeting.

In some embodiments of these devices and all such devices described herein, the substrate is configured to move perpendicular to the direction of the collecting electrode when the device is in use. In some embodiments of these devices and all such devices described herein, the substrate is arranged on one or more rollers to permit the substrate to move perpendicular to the direction of the collecting electrode when the device is in use.

In some embodiments of these devices and all such devices described herein, the collecting electrode is substantially parallel to the electrospinning electrode.

In some embodiments of these devices and all such devices described herein, the electrospinning electrode comprises a charged surface from which fibers are electrospun, such that the length of the gap of exposed collecting electrode is aligned with and substantially the same length or less than the charged surface of the electrospinning electrode from which fibers are electrospun.

Also provided herein, in some aspects, are nozzle-less electrospinning devices, such devices comprising:
an electrospinning electrode and a collecting electrode comprising connections for a DC power supply,
the electrospinning electrode and the collecting electrode spaced apart and establishing an electric field between the electrospinning electrode and the collecting electrode when DC power is supplied;
   the collecting electrode comprising a first end and a second end;
   the electrospinning electrode comprising a continuously fed or static charged electrode member partially submerged in or carrying an entrained polymer solution to permit electrospinning in a polymer solution to permit electrospinning fibers of the polymer towards the collecting electrode;
   a substrate located between the electrospinning electrode and the collecting electrode such that electrospun polymer fibers become deposited on the substantially planar substrate when in use;
   a first shield comprised of a first insulating material member situated between the substrate and the collecting electrode and extending from the first end of the collecting electrode towards the second end of the collecting electrode such that a portion of the electric field is shielded by the first insulating material member and a gap of unshielded collecting electrode is formed extending from an end of the first insulating material member towards the second end of the collecting electrode;
   such that the first shield increases the uniform area of an electrospun polymer mat deposited on the substrate relative to the uniform area of a polymer mat deposited in the absence of the first shield.

In some embodiments of these devices and all such devices described herein, the device further comprises a second shield comprised of a second insulating material member situated between the substrate and the collecting electrode and extending from the second end of the collecting electrode towards the first end of the collecting electrode, such that a portion of the electric field is shielded by the second insulating material member and the gap of unshielded collecting electrode extends between an end of the first insulating material member and an end of the second insulating material member, such that the second shield further increases the uniform area of an electrospun polymer mat deposited on the substrate relative to the uniform area of a polymer mat deposited in the absence of the second shield.

In some embodiments of these devices and all such devices described herein, the device further comprises a first collimating shield comprised of a third insulating material member, the first collimating shield supported and situated adjacent to the substrate and between the substrate and the electrospinning electrode, the first collimating shield extending substantially perpendicular to the collecting electrode, an edge of the first collimating shield facing the gap of unshielded collecting electrode aligned with the end of the first shield insulating material member adjacent the gap of unshielded collecting electrode, such that the first collimating shield increases the uniform area of an electrospun polymer mat deposited on the substrate relative to the uniform area of a polymer mat deposited in the absence of the collimating shield.

In some embodiments of these devices and all such devices described herein, the device further comprises a second collimating shield comprised of a fourth insulating material member, the second collimating shield supported and situated adjacent to the substrate and between the substrate and the electrospinning electrode, the second collimating shield extending substantially perpendicular to the collecting electrode, an edge of the second collimating shield facing the gap of unshielded collecting electrode aligned with the end of the first shield insulating material member adjacent the gap of unshielded collecting electrode, such that the first collimating shield increases the uniform area of an electrospun polymer mat deposited on the substrate relative to the uniform area of a polymer mat deposited in the absence of the collimating shield.

In some embodiments of these devices and all such devices described herein, the device further comprises a first encircling insulating material member encircling the collecting electrode and extending along the collecting electrode from the first end of the collecting electrode towards the second end of the collecting electrode such that a portion of the electrode is covered by the first encircling insulating material member and a gap of exposed collecting electrode is formed extending from an end of the first encircling insulating material member towards the second end of the collecting electrode.

In some embodiments of these devices and all such devices described herein, the device further comprises a second encircling insulating material member encircling the collecting electrode and extending along the collecting electrode from the second end of the collecting electrode towards the first end of the collecting electrode such that a portion of the electrode is covered by the second encircling insulating material member and a gap of exposed collecting electrode is defined extending from an end of the first encircling insulating material member to an end of the second encircling insulating material member.

In some embodiments of these devices and all such devices described herein, the first shield has a dielectric constant of at least 1.2.

In some embodiments of these devices and all such devices described herein, the first shield comprises a material selected from the group consisting of rubber, glass, cotton, perlite, charcoal, wood, fiberglass, fiberglass insulation, polyethylene, high density polyethylene, low density polyethylene, polypropylene, polystyrene and foamed versions of polyethylene, polypropylene and polystyrene, polyvinyl alcohol and polytetra fluoroethylene. In some embodiments of these devices and all such devices described herein, the first shield comprises polyethylene foam.

In some embodiments of these devices and all such devices described herein, the second shield has a dielectric constant of at least 1.2.

In some embodiments of these devices and all such devices described herein, the second shield comprises a material selected from the group consisting of rubber, glass, cotton, perlite, charcoal, wood, fiberglass, fiberglass insulation, polyethylene, high density polyethylene, low density polyethylene, polypropylene, polystyrene and foamed versions of polyethylene, polypropylene and polystyrene, polyvinyl alcohol and polytetra fluoroethylene. In some embodiments of these devices and all such devices described herein, the second shield comprises polyethylene foam.

In some embodiments of these devices and all such devices described herein, the collimating shield has a dielectric constant of at least 1.2.

In some embodiments of these devices and all such devices described herein, the collimating shield comprises a material selected from the group consisting of rubber, glass, cotton, perlite, charcoal, wood, fiberglass, fiberglass insulation, polyethylene, high density polyethylene, low density polyethylene, polypropylene, polystyrene and foamed versions of polyethylene, polypropylene and polystyrene, polyvinyl alcohol and polytetra fluoroethylene. In some embodiments of these devices and all such devices described herein, the collimating shield comprises polyethylene foam.

In some embodiments of these devices and all such devices described herein, the encircling, insulating material has a dielectric constant of at least 1.2.

In some embodiments of these devices and all such devices described herein, the encircling, insulating material comprises a material selected from the group consisting of rubber, glass, cotton, perlite, charcoal, wood, fiberglass, fiberglass insulation, polyethylene, high density polyethylene, low density polyethylene, polypropylene, polystyrene and foamed versions of polyethylene, polypropylene and polystyrene, polyvinyl alcohol and polytetra fluoroethylene. In some embodiments of these devices and all such devices described herein, the encircling, insulating material comprises polyethylene foam.

In some embodiments of these devices and all such devices described herein, the uniform area of the electrospun polymer mat deposited on the substrate by the device in the presence of the first, and preferably second shield member(s) is at least 25 cm (e.g., in the “cross-direction” or “carriage direction” (CD) dimension) by 100 cm (e.g., in the “machine direction” (MD) dimension).

In some embodiments of these devices and all such devices described herein, the substrate is substantially planar.

In some embodiments of these devices and all such devices described herein, the substrate is waxed paper.

In some embodiments of these devices and all such devices described herein, the substrate is configured to move perpendicular to the direction of the collecting electrode when the device is in use. In some embodiments of these devices and all such devices described herein, the substrate is arranged on one or more rollers to permit the substrate to move perpendicular to the direction of the collecting electrode when the device is in use.

In some embodiments of these devices and all such devices described herein, the collecting electrode is substantially parallel to the electrospinning electrode.

In some embodiments of these devices and all such devices described herein, the electrospinning electrode comprises a charged surface from which fibers are electrospun, such that the length of the gap of exposed collecting electrode is aligned with and substantially the same length as the charged surface of the electrospinning electrode from which fibers are electrospun.

Also provided herein, in some aspects, are uniform high basis weight, non-woven, polymer nanofiber fabric compositions.

In some embodiments of these compositions and all such compositions described herein, the nanofiber non-woven fabric composition is uniform over an area of at least 25 cm (e.g., in the CD dimension) by at least 100 cm (e.g., in the MD dimension).

In some embodiments of these compositions and all such compositions described herein, the weight of any 1 cm disc obtained from the area of at least 25 cm (e.g., in the CD dimension) by at least 100 cm (e.g., in the MD dimension) is within 10% of the mean basis weight over the entire area of at least 25 cm by at least 100 cm.

In some embodiments of these compositions and all such compositions described herein, the basis weight is in the range of 50-500 gm/m², inclusive.

In some embodiments of these compositions and all such compositions described herein, the nanofiber non-woven fabric composition is produced by an electrospinning method. In some embodiments of these compositions and all such compositions described herein, the electrospinning is performed using a nozzle-less electrospinning method.

In some embodiments of these compositions and all such compositions described herein, the polymer is rapidly water soluble. In some embodiments of these compositions and all such compositions described herein, the rapidly water soluble polymer provides burst biologically active agent release. In some embodiments of these compositions and all such compositions described herein, the rapidly water soluble polymer is selected from polyvinyl alcohol, polyethylene oxide, polyvinylpyrrolidone, poly-2-ethyl-2-oxazoline, and polyacrylic acid.

In some embodiments of these compositions and all such compositions described herein, the polymer provides sustained biologically active agent release.

In some embodiments of these compositions and all such compositions described herein, the polymer is selected from poly[lactic-co-glycolic] acid, polycaprolactone, and ethyl cellulose.

In other aspects, provided herein are biologically active agent-delivery compositions comprising uniform high basis weight, non-woven, polymer nanofiber fabric compositions.

In some embodiments of these compositions and all such compositions described herein, the nanofiber non-woven fabric comprises a uniform distribution of one or more biologically active agents.

In some embodiments of these compositions and all such compositions described herein, the nanofiber non-woven fabric composition comprises at least 5-60% by weight of the one or more biologically active agents.

In some embodiments of these compositions and all such compositions described herein, the compositions comprise a uniform distribution of at least two biologically active agents.

In some embodiments of these compositions and all such compositions described herein, the one or more biologically active agents are selected from tenofovir, dapivirine, levonorgestrel, etravirine, raltegravir, and maraviroc.

In some embodiments of these compositions and all such compositions described herein, the biologically active agents are electrospun in different solid states, for example, where one is a crystalline solid dispersion and the other is molecularly dispersed.

In some embodiments of these compositions and all such compositions described herein, the two or more biologically active agents are selected from tenofovir, dapivirine, levonorgestrel, etravirine, raltegravir, and maraviroc.

Also provided herein, in some aspects, are composite biologically active agent-delivery compositions comprising a first layer of uniform high basis weight, non-woven, polymer nanofiber fabric composition comprising a first biologically active agent, and a second layer of uniform high basis weight, non-woven, polymer nanofiber fabric composition comprising a second biologically active agent, such that the polymer is the same in the first and second layers.

In some embodiments of these compositions and all such compositions described herein, each of the nanofiber non-woven fabric compositions is uniform over an area of at least 25 cm (e.g., in the CD dimension) by at least 100 cm (e.g., in the MD dimension).

In some embodiments of these compositions and all such compositions described herein, the weight of any 1 cm disc obtained from the area of at least 25 cm (e.g., in the CD dimension) by at least 100 cm (e.g., in the MD dimension) is within 10% of the mean basis weight over the entire area of at least 25 cm by at least 100 cm.

In some embodiments of these compositions and all such compositions described herein, the basis weight is in the range of 50-500 gm/m², inclusive.

In some embodiments of these compositions and all such compositions described herein, at least one of the nanofiber non-woven fabric compositions is produced by an electrospinning method. In some embodiments of these compositions and all such compositions described herein, the electrospinning is performed using a nozzle-less electrospinning method.

In some embodiments of these compositions and all such compositions described herein, the polymer is rapidly water soluble. In some embodiments of these compositions and all such compositions described herein, the rapidly water soluble polymer provides burst biologically active agent release of the first and second biologically active agents. In some embodiments of these compositions and all such compositions described herein, the rapidly water soluble polymer is selected from polyvinyl alcohol, polyethylene oxide, polyvinylpyrrolidone, poly-2-ethyl-2-oxazoline, and polyacrylic acid.

In some embodiments of these compositions and all such compositions described herein, the polymer provides sustained biologically active agent release. In some embodiments of these compositions and all such compositions described herein, the polymer is selected from poly[lactic-co-glycolic] acid, polycaprolactone, and ethyl cellulose.

In some embodiments of these compositions and all such compositions described herein, the first and second biologically active agents are selected from tenofovir, dapivirine, levonorgestrel, etravirine, raltegravir, and maraviroc.

In some aspects, provided herein are composite biologically active agent-delivery compositions comprising a first layer of uniform high basis weight, non-woven, polymer nanofiber fabric composition comprising a first biologically active agent, and a second layer of uniform high basis weight, non-woven, polymer nanofiber fabric composition comprising a second biologically active agent, such that the polymer is different in the first and second layers.

In some embodiments of these compositions and all such compositions described herein, each of the nanofiber non-woven fabric compositions is uniform over an area of at least 25 cm (e.g., in the CD dimension) by at least 100 cm (e.g., in the MD dimension).

In some embodiments of these compositions and all such compositions described herein, the weight of any 1 cm disc obtained from the area of at least 25 cm (e.g., in the CD dimension) by at least 100 cm (e.g., in the MD dimension) is within 10% of the mean basis weight over the entire area of at least 25 cm by at least 100 cm.

In some embodiments of these compositions and all such compositions described herein, the basis weight of each layer is in the range of 50-500 gm/m², inclusive.

In some embodiments of these compositions and all such compositions described herein, at least one of the nanofiber non-woven fabric compositions is produced by an electrospinning method. In some embodiments of these compositions and all such compositions described herein, the electrospinning is performed using a nozzle-less electrospinning method.

In some embodiments of these compositions and all such compositions described herein, either or both of the different polymers is/are rapidly water soluble. In some embodiments of these compositions and all such compositions described herein, the rapidly water soluble polymer provides burst biologically active agent release of the first and second biologically active agents. In some embodiments of these compositions and all such compositions described herein, the rapidly water soluble polymer is selected from polyvinyl alcohol, polyethylene oxide, polyvinylpyrrolidone, poly-2-ethyl-2-oxazoline, and polyacrylic acid.

In some embodiments of these compositions and all such compositions described herein, either or both of the polymers provide(s) sustained biologically active agent release. In some embodiments of these compositions and all such compositions described herein, the polymer is selected from poly[lactic-co-glycolic] acid, polycaprolactone, and ethyl cellulose.

In some embodiments of these compositions and all such compositions described herein, the first layer polymer is rapidly water soluble and the second layer polymer provides sustained biologically active agent release. In some embodiments of these compositions and all such compositions described herein, the rapidly water soluble polymer provides burst biologically active agent release of the first biologically active agent.

In some embodiments of these compositions and all such compositions described herein, the rapidly water soluble polymer is selected from polyvinyl alcohol, polyethylene oxide, polyvinylpyrrolidone, poly-2-ethyl-2-oxazoline, and polyacrylic acid.

In some embodiments of these compositions and all such compositions described herein, the second layer polymer is selected from poly[lactic-co-glycolic] acid, polycaprolactone, and ethyl cellulose.

In some embodiments of these compositions and all such compositions described herein, The first and second biologically active agents are selected from tenofovir, dapivirine, levonorgestrel, etravirine, raltegravir, and maraviroc.

In some embodiments of these compositions and all such compositions described herein, the compositions are fabricated using methods of electrospinning comprising electrospinning fibers from a solution comprising a polymer dissolved in a solvent. In some embodiments of these compositions and all such compositions described herein, the method comprises nozzle-less electrospinning

In some embodiments of these compositions and all such compositions described herein, the nanofiber non-woven fabric composition is fabricated using any of the devices described herein.

In some embodiments of these compositions and all such compositions described herein, the solvent is selected from tetrahydrofuran, trifluoroethanol, dimethyl sulfoxide, dimethylformamide, dichloromethane, ethanol, methanol, isopropanol, hexafluoroisopropanol, chloroform and water.

Also provided herein, in some aspects, are methods of producing a uniform high basis weight, non-woven, polymer nanofiber fabric, biologically active agent-delivery composition, the method comprising electrospinning fibers from a solution comprising a polymer and one or more biologically active agents from a nozzle-less electrospinning device.

In some embodiments of these methods and all such methods described herein, the nanofiber non-woven fabric, biologically active agent-delivery composition is uniform over an area of at least 25 cm (e.g., in the CD dimension) by at least 100 cm (e.g., in the MD dimension).

In some embodiments of these methods and all such methods described herein, the weight of any 1 cm disc obtained from the area of at least 25 cm (e.g., in the CD dimension) by at least 100 cm (e.g., in the MD dimension) is within 10% of the mean basis weight over the entire area of at least 25 cm by at least 100 cm.

In some embodiments of these methods and all such methods described herein, the basis weight of the nanofiber non-woven fabric, biologically active agent-delivery composition is in the range of 50-500 gm/m², inclusive.

In some embodiments of these methods and all such methods described herein, the nanofiber non-woven fabric, biologically active agent-delivery composition comprises at least 5-60% by weight of the one or more biologically active agents.

In some embodiments of these methods and all such methods described herein, the nozzle-less electrospinning device is any of the devices described herein.

In some aspects, provided herein are methods of administering a biologically active agent to a subject, the method comprising contacting any of the biologically active agent-delivery compositions described herein with a tissue, organ, or other surface or cavity of a subject in need thereof.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

As used herein, “modulating” or “to modulate” generally means either reducing or increasing a desired outcome or desired parameter using the compositions, methods, or devices described herein compared to the outcome or parameter under the same conditions when not using the compositions, methods, or devices described herein. An “increase” or “decrease” refers to a statistically significant increase or decrease, respectively. For the avoidance of doubt, an increase or decrease will be at least 10% relative to a reference, such as at least 10%, at least 20%, at least 30%, at least 40%, at least 50%,a t least 60%, at least 70%, at least 80%, at least 90%, at least 95%, at least 97%, at least 98%, or more, up to and including at least 100% or more, inclusive, and in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more.

The term “statistically significant” or “significantly” refers to statistical significance and generally means a two standard deviation (2SD) or greater difference in the subject parameter. The term refers to statistical evidence that there is a difference. It is defined as the probability of making a decision to reject the null hypothesis when the null hypothesis is actually true. The decision is often made using the p-value.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows unrestrained lines of electric field between spinning and collecting electrodes of a free-surface, needle-less or nozzle-less electrospinning device. As demonstrated herein. undesirable lines can be removed or prevented to focus nanofiber distribution more uniformly in the cross-direction.

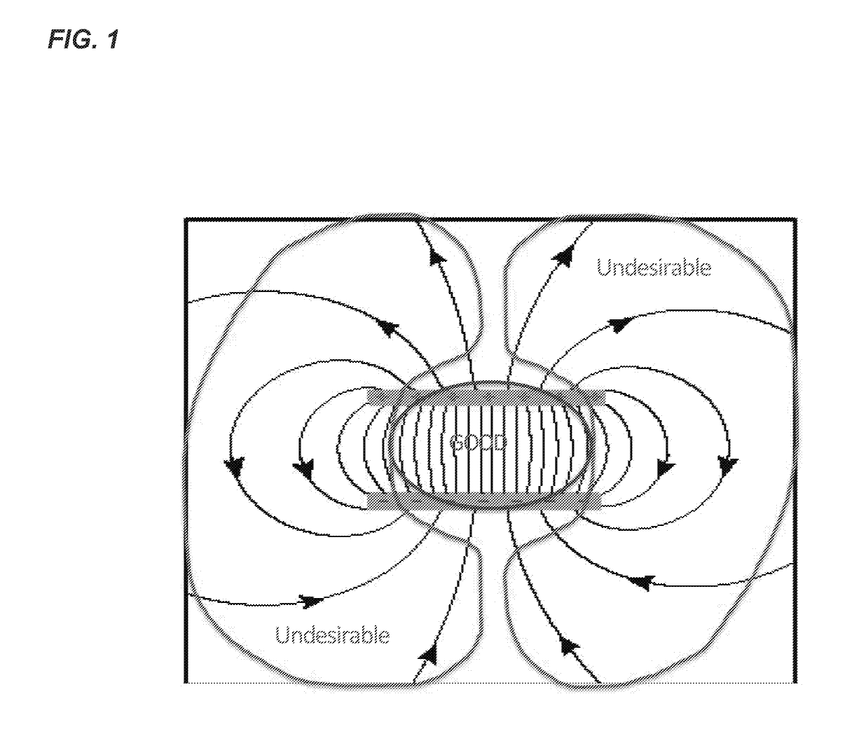

FIG. 2 shows an exemplary model to create controlled and directed electric field lines for uniform fiber deposition by employing shielding and insulation.

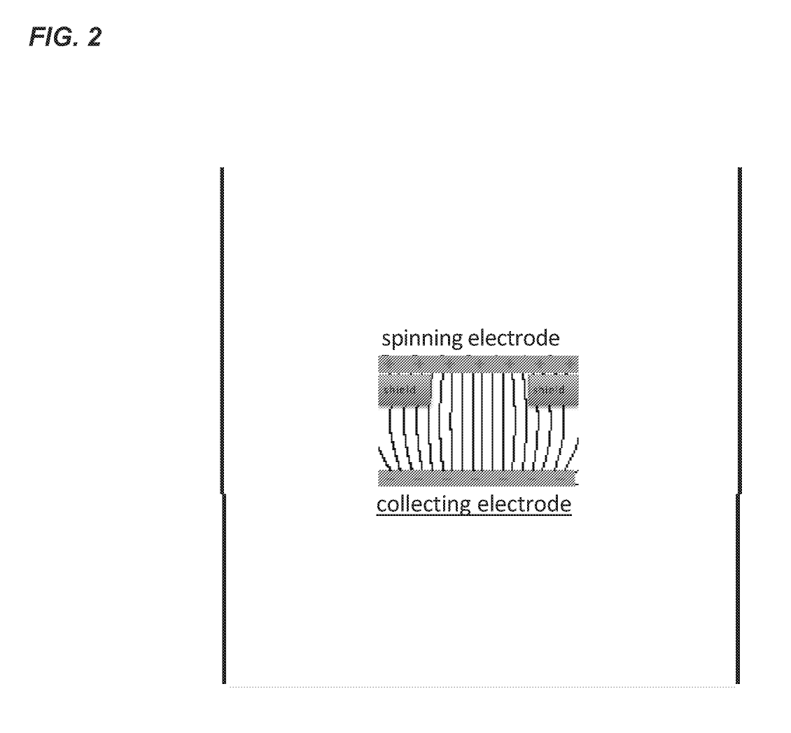

FIG. 3 shows that diverging field lines contribute to normal distribution of electrospun fibers, which need to be altered to create a more uniform, non-normal distribution. Collecting electrode, (CE) 10, substrate 20, electrospinning electrode (SE) 30, polymer film 40 on the SE 30 and nanofiber trajectories 50 are shown.

FIG. 4 shows a typical cross-direction (CD) mass profile of nanofibers using a 25 cm carriage length.

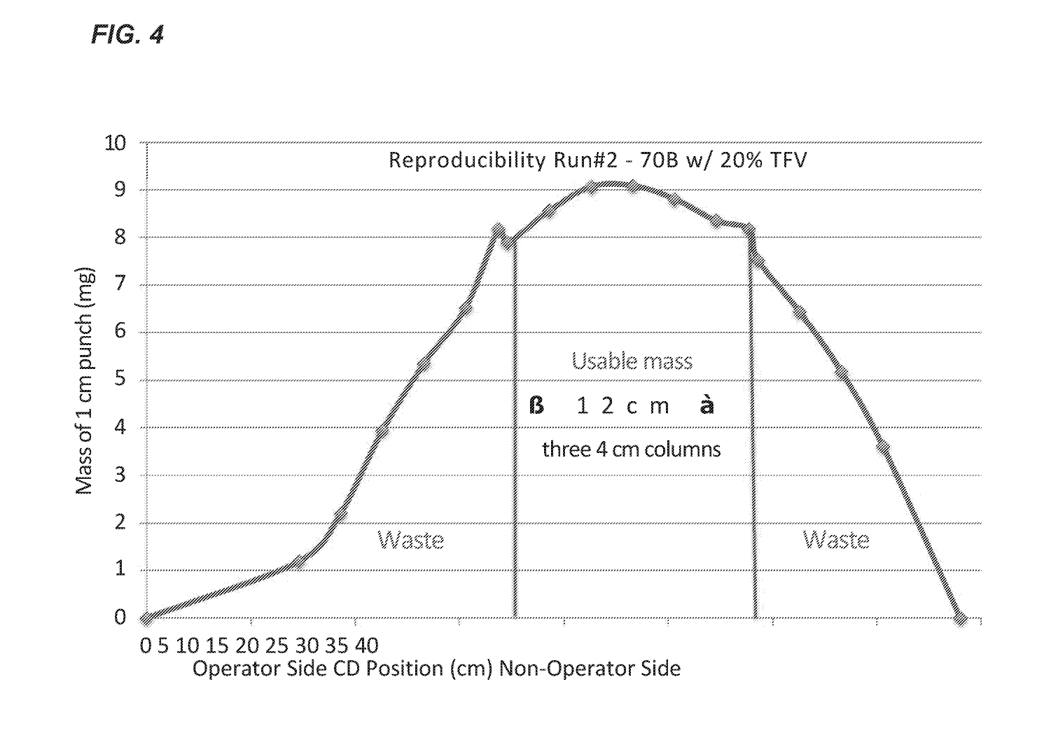

FIG. 5 shows an embodiment of a NANOSPIDER™ needle-less, nozzle-less or free-surface electrospinning device and standard electrospinning conditions when using it.

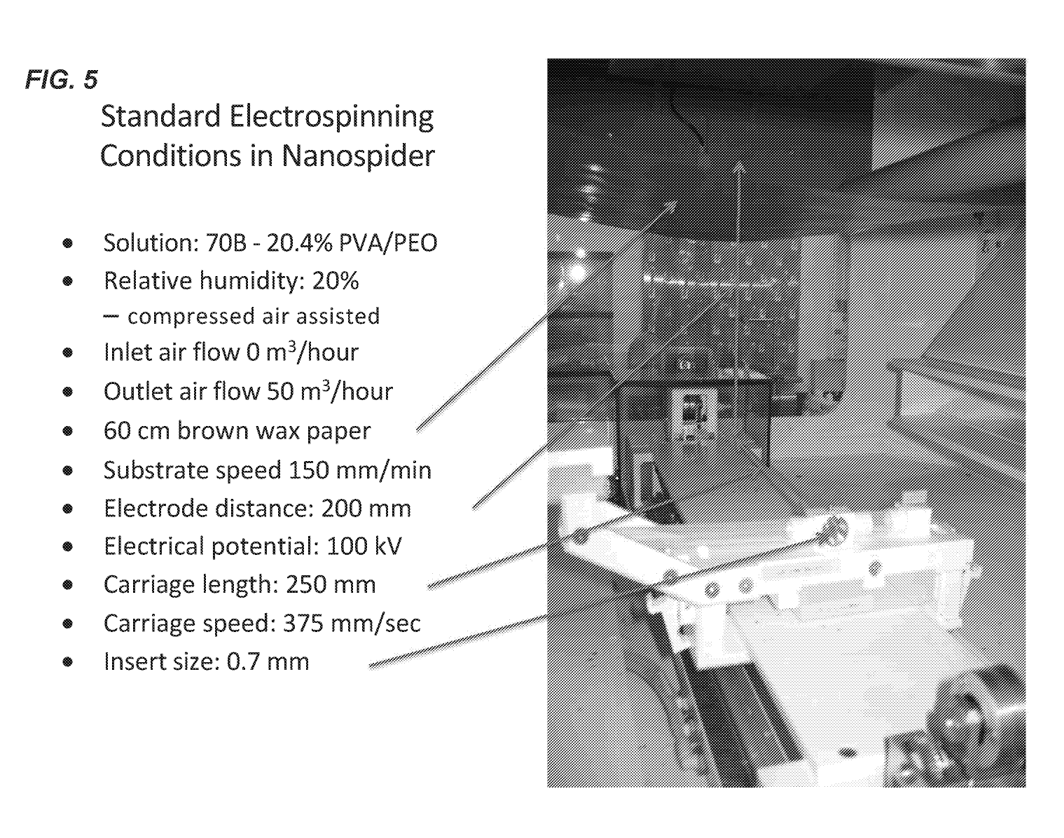

FIGS. 6A-6B show an embodiment in which an insulating material member 60 encircling a portion of the CE 10 provides for increased uniformity of non-woven, nanofiber electrospun polymer fabric. FIG. 6A. Insulating material member encircling a portion of the CE. FIG. 6B. Distribution of non-woven, nanofiber electrospun polymer fabric 70 deposited on a waxed paper substrate 20 using the insulating member arrangement shown in FIG. 6A.

FIGS. 7A-7B show an embodiment in which shielding 80a and 80b comprised of an insulating material placed between the substrate 20 and CE 10 shields a portion of the electric field and increases the uniformity of the resulting non-woven, nanofiber electrospun polymer fabric 70. FIG. 7A. 1″ thick polyethylene (PE) foam blocks 80a, 80b laid on top (back side) of brown wax paper substrate 20 below the CE with butyl rubber insulation 60. FIG. 7B. Distribution of non-woven, nanofiber electrospun polymer fabric 70 deposited on a waxed paper substrate 20 using the insulating member arrangement shown in FIG. 7A.

FIG. 8 demonstrates a dramatic change in cross direction (CD) mass profile when polyethylene block shielding and butyl rubber CE insulation were used.

FIG. 9A-9B show an embodiment including polystyrene foam shielding shaped to accommodate a CE with butyl rubber BR insulation in CE housing. FIG. 9A. Polystyrene (PS) foam shield shaped to accommodate BR-insulated CE. FIG. 9B. Shows non-woven, nanofiber electrospun polymer fabric results in one embodiment using BR insulation also added to SE having 25 cm gap, and notes that overspray of fibers beyond CD limits is not desirable.

FIG. 10 shows a CD mass profile for the embodiment of FIGS. 9A-9B, which used polystyrene (PS) foam shielding. While demonstrating an improvement over non-shielded electrospinning, the results indicate that polystyrene foam is less effective than polyethylene foam.

FIGS. 11A-11B show an embodiment in which polystyrene (PS) foam is used to shield electrical leads to the CE. FIG. 11A. PS foam shielding of the electrical leads to the CE. FIG. 11B. Shows non-woven, nanofiber electrospun polymer fabric resulting from the shielding arrangement shown in FIG. 11A.

FIG. 12 demonstrates that while polystyrene (PS) foam provides an improvement over a lack of shielding, the CD mass profile and fall-off rate is better with polyethylene (PE) foam than polystyrene (PS) foam.

FIG. 13 shows an investigation of CD profile with different carriage speed. A slower carriage speed of 250 mm/sec, which changes residence time and shear, affected mass distribution. The 375 mm/sec carriage speed provided improved CD profile relative to the slower carriage speed.

FIGS. 14A-14B show an embodiment in which the effects of additional CE electrical connections and shielding thereof were investigated for their effects on electric field and nanospun fabric uniformity in CD.

FIG. 15 demonstrates improved CD mass uniformity with a second CE electrical connection. The embodiment examined included polyethylene shielding, CE butyl rubber insulation, and a 2nd CE electrical connection.

FIG. 16 shows an embodiment including addition of second CE electrical connections.

FIG. 17 shows the results of experiments examining the effect of air flow on CD mass variability.

FIG. 18 demonstrates uniformity returned with typical air flow-50 m³/hour using polyethylene shielding, CE butyl rubber insulation, and a dual CE and SE connector.

FIG. 19 demonstrates that at high basis weight (BW), there is good CD mass standard deviation with broad plateau (20 cm) using an embodiment including polyethylene shielding, CE butyl rubber insulation, and a dual CE and SE connector.

FIG. 20 demonstrates that adding tenofovir (TFV) tends to improve uniformity.

FIG. 21 depicts an investigation of using intentionally unequal carriage length with CE gap. Current unshielded CE 10 length is 22 cm with a 25 cm carriage length. It was proposed that increasing carriage length to 33 cm and might further force fibers to accumulate at CD ends of CE 10 increasing fall-off rate and that an increased carriage speed could favor plateau shaped profile of fiber accumulation. Shield 80 and insulating material member 60 (hidden by shield 80) are indicated, as is SE 30.

FIG. 22 shows non-woven, electrospun nanofiber polymer fabric results of a static run with SE>>CE to force fibers to CD ends aided by 480 mm/sec carriage speed.

FIG. 23 shows the CD mass profile for fabric made using the design and run parameters noted for FIG. 21. The approach forced fibers to CD edges, but increased variability.

FIG. 24 shows an embodiment of the CE housing with polyethylene and polystyrene shielding.

FIG. 25 shows results when CE and SE length is increased to 33 cm from 22 and 25 cm.

FIG. 26 shows that a higher carriage speed can produce more variable results.

FIG. 27 shows results from using five 4 cm columns. 162 4×4 cm samples were used where X=99 gsm, COV=2.3%. 5 columns were used in MD using a 33 cm carriage length giving a yield of mass w/i 90% Max BW/total mass =57%. These results were a lower BW than Run #3 (FIG. 22) and less contiguous samples. Loading efficiency: X=19.8 mg TFV/100 mg fiber. Encapsulation efficiency: X=99%, COV=1.6%.

FIGS. 28A-28B show a set-up designed to further focus the electric field. Theoretically, a 33 cm carriage distance should yield six 4×4 cm columns with current fall-off rate and no CD fringe fibers. An embodiment was tested using vertical PE shields aligned exactly at edge of existing CE polyethylene shields and butyl rubber insulation that serves as an electric field barrier deflecting lines, not a physical barrier.

FIG. 29 depicts an embodiment making the CE gap equal to carriage length and adding PE foam collimating side shields at edges. CE 10, Substrate 20, SE 30, polymer film 40, electrospinning trajectories 50, and CE shielding 80a and 80b are as shown in earlier figures. Collimating shielding 90a and 90b is as indicated.

FIG. 30 shows 8 machine-direction (MD) passes with PE foam CD collimating side shields, with CE PE shields, BR CE insulation, and dual SE and CE connections.

FIG. 31 shows 20 MD passes with PE foam CD collimating side shields, with CE PE shields, BR CE insulation, and dual SE and CE connections.

FIG. 32 shows an investigation of why fibers collect on the face of cross direction PE collimating side shields.

FIG. 33 depicts optimizing CE gap to actual carriage length.

FIG. 34 shows a favorable fiber footprint and no fringe fibers, demonstrating that PE CD collimating side shields are effective.

FIG. 35 shows results using a 70B polymer blend formulation comprised of 14 wt/wt % of 400 kDa polyethylene oxide and 86% wt/wt 50 kDa polyvinyl alcohol, which is typically made into a 20% wt/vol solution in water for electrospinning, without shielding and a 25 cm carriage width.

FIG. 36 shows results using a 70B polymer blend formulation, without shielding and a 25 cm carriage width.

FIG. 37 shows optimizing CE gap to actual carriage length and using CD collimating side shields.

FIG. 38 shows performance of monthly maintenance (clean, lube & adjust carriage) to address variability and low non-operator side shoulder using CE PE shield, CD PE shield, dual SE & CE connections in place.

FIG. 39 shows results of performance of monthly maintenance using CE PE shield, CD PE shield, and dual SE & CE connections in place.

FIG. 40 shows modifications based on observation that carriage contacting carriage platform may contribute to low non-op side shoulder, such that carriage platform was shimmed to prevent carriage and platform contact and impact on carriage speed was removed.

FIG. 41 shows results from shimmed carriage platform using CE PE shields, CD PE foam side shields, BR CE insulation and dual SE & CE connections in place.

FIG. 42 shows results from six MD columns of 4×4 cm samples cut.

FIG. 43 shows results using six columns of 4×4 cm using 35 cm carriage distance.

FIG. 44 shows that careful measurements show that PE shims slightly increase electrode distance on non-operator side, ˜1 cm. Shims removed from both ends of CE and may be contributing to low shoulder.

FIG. 45 demonstrates that PE shim removal equalizes both shoulders using PE foam CD side shields with CE PE shields, BR CE insulation and dual SE CE connections.

FIGS. 46A-46C show that raltegravir (RAL) exhibits more variable distribution within RAL/miraviroc (MVC)/etravirine (ETR) triple drug fibers.

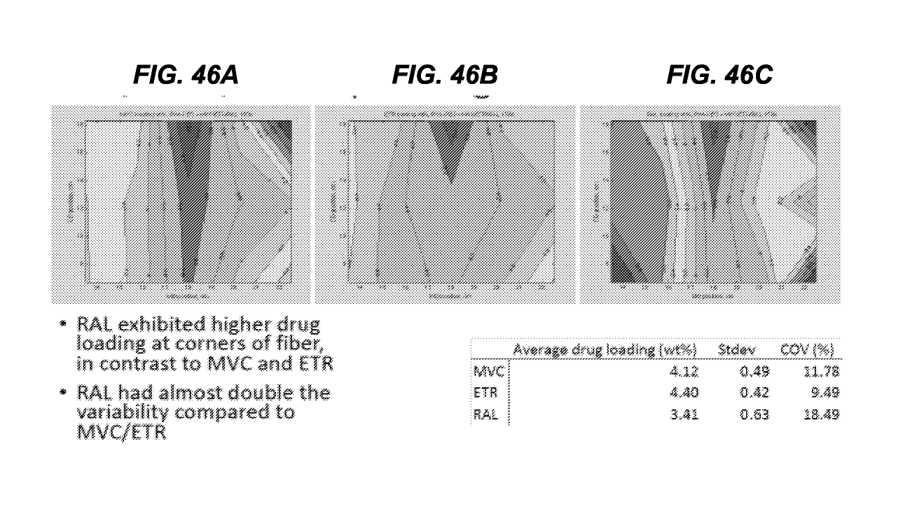

FIGS. 47A-47B show MVC/ETR only fibers exhibit similar coefficient of variance (COV) to triple drug fibers.

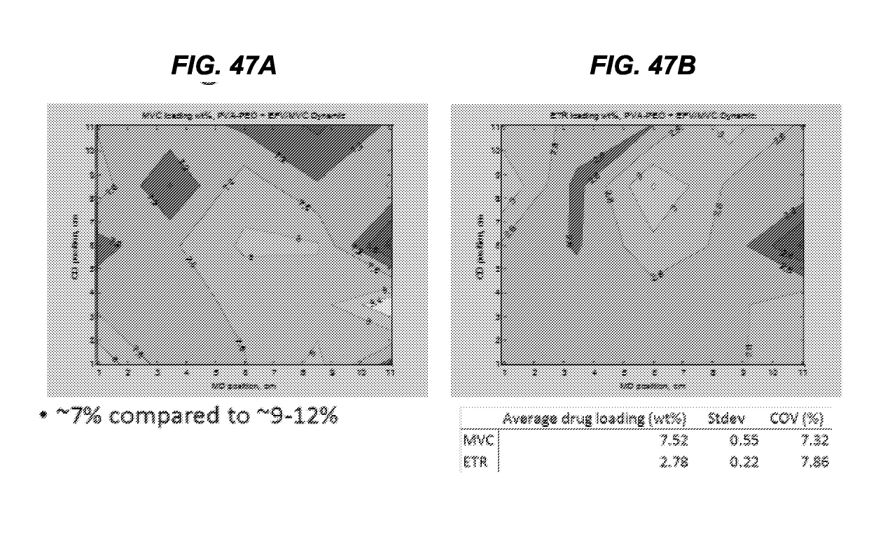

FIG. 48 shows RAL only fibers exhibit similar COV to RAL in triple drug fibers. Results show 17% compared to 18.5% in the triple combination, indicating that individual properties of RAL, and not drug interactions, are causing increased variability.

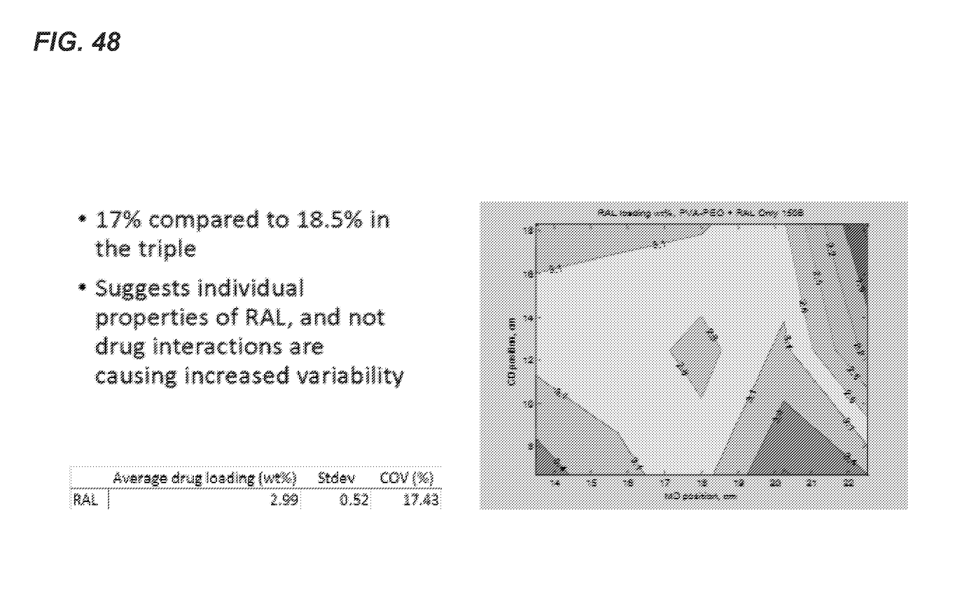

FIG. 49 demonstrates that solubilized RAL shows improved distribution. RAL was solubilized using NaOH. Solubilized RAL showed improved drug distribution (10% vs. 17/18%).

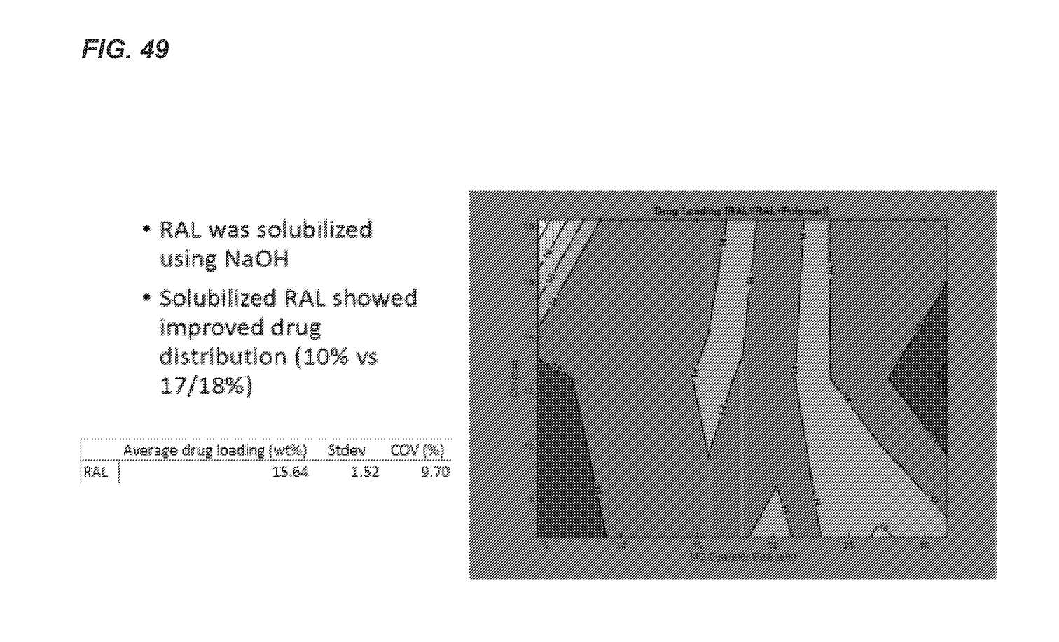

FIGS. 50A-50C demonstrate that solubilized RAL layer exhibited low variability in comparison to MVC/ETR. Dual layer fiber was spun, where RAL was solubilized first, followed by an MVC/ETR layer spun over the RAL. Solubilized RAL showed low variability in this format, similar to solubilized RAL alone. MVC exhibited higher variability due to homogenization issues.

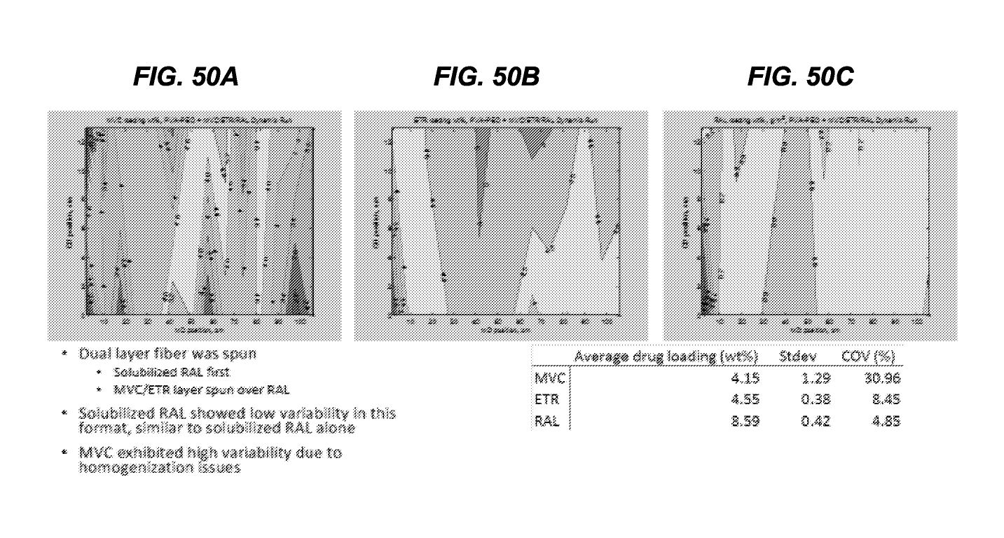

FIGS. 51A-51D show an embodiment using the optimization modules described herein to determine response indicators of productivity and uniformity.

FIGS. 52A-52D show an embodiment using the optimization modules described herein to determine response indicators of productivity and uniformity.

FIGS. 53A-53D show an embodiment using the optimization modules described herein to determine response indicators of productivity and uniformity.

FIGS. 54A-54D show an embodiment using the optimization modules described herein to determine response indicators of productivity and uniformity.

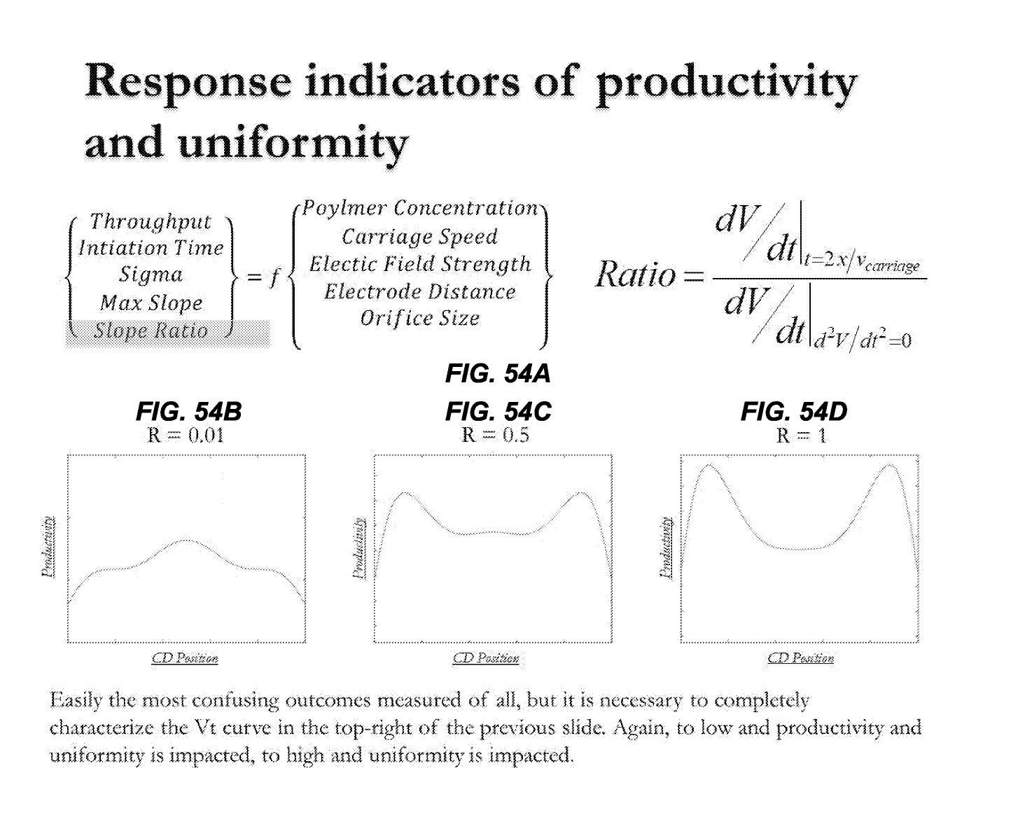

FIGS. 55A-55C show an embodiment using the optimization modules described herein to determine productivity and uniformity from a static wire electrode.

FIGS. 56A-56D show optimization module outcomes for various material polymer compositions.

FIG. 57 compares the results obtained using the optimization modules, video prediction, and actual empirically determined results.

### DETAILED DESCRIPTION

Provided herein are novel compositions, methods, and devices to increase nanofiber fabric yields, basis weights, and uniformity for scalable manufacturing of nanofibers by methods such as electrospinning

Electrospinning is a process for forming fibers, including nanofibers, through the action of electrostatic forces. When the electrical force at the interface of a polymer solution overcomes surface tension, a charged jet is ejected. The jet initially extends in a straight line, then undergoes various whipping motions during the flight from nozzle to collector. As it reaches a grounded target, the jet stream can be collected as an interconnected web of fine sub-micron size fibers. The polymer is commonly collected onto a grounded mesh or plate in the form of a nonwoven mat of high surface area. The resultant fibers have a fine thickness, ranging from micron-scale diameter to nano-scale. Polymer nanofibers, possessing high surface area to mass ratios, have great use in a variety of applications in a wide variety of fields, including filter media, tissue-engineering scaffold structures and devices, nanofiber-reinforced composite materials, sensors, electrodes for batteries and fuel cells, catalyst support materials, wiping cloths, absorbent pads, post-operative adhesion preventative agents, smart-textiles, as well as in artificial cashmere and artificial leather.

Large-scale electrospinning is typically done using multi-nozzle electrospinning methods with the use of multi-nozzle devices, for example as described in WO2005/073441, the contents of which are hereby incorporated by reference in their entirety, and via nozzle-free electrospinning methods with the use of nozzle free devices, for example using a NANOSPIDER™ apparatus, bubble-spinning or the like; or via electroblowing, for example as described in WO03/080905, the contents of which are hereby incorporated by reference in their entirety. In a nozzle-free or nozzle-less process, no spinneret with nozzles is present, and another device is present to which the solution is fed, and from which the jet streams are formed. For example, the solution can be entrained on a rotating electrode, or use a wire passing through a small orifice (0.5-0.8 mm) as in a NANOSPIDER™ apparatus, or bubbles can be formed from the solution by purging gas through the solution (as in bubble spinning) In a nozzle-free process, the solution fed by such means produces a series of Taylor cones under the influence of the high voltage, such as between 30-120 kVolts. From these Taylor cones, charged jet streams are formed above a critical voltage that end up as the nanofibers.

One embodiment of a basic nozzle-less electrospinning apparatus comprises a rotating surface, such as a rotating drum dipped into a bath of polymer solution, where the thin layer of polymer is carried on the drum surface and exposed to a high voltage electric field. Another embodiment uses a wire passing through a small orifice (e.g., 0.5-0.8 mm, as noted above) that is continuously fed with polymer. In either instance, if the voltage exceeds the critical value, a number of electrospinning jets are generated. The jets are distributed over the electrode surface with a mathematically determined periodicity. This is one of the main advantages of nozzle-less electrospinning: the number and location of the jets is set up naturally in their optimal positions. See, for example, U.S. Pat. No. 6,743,273, Patent Application Nos. US2006/0290031, and WO2006/131081, the contents of which are herein incorporated by reference in their entireties. Other “nozzle-less” or “needle-less” free surface approaches to continuously feed polymer solution to an electrospinning electrode are known to those of skill in the art.

When using a nozzle-less electrospinning apparatus, first, a polymer material is dissolved in a solvent until completely dissolved—this can be, for example, overnight or over multiple days. Solvents suitable for electrospinning can be selected by the ordinarily skilled artisan on the basis of solubility of the selected polymer and any biologically active agent(s) to be included in the electrospun fiber compositions. Examples of solvents include, but are not limited to tetrahydrofuran, trifluoroethanol, dimethyl sulfoxide, dimethylformamide, dichloromethane, ethanol, methanol, isopropanol, hexafluoroisopropanol, chloroform, acetic acid, formic acid, trifluoracetic acid, trichloracetic acid, acetone, and water. Once the chosen polymer is/are dissolved, any active ingredients can be added, as well as any additives to affect solution properties, such as, for example, viscosity, surface tension, pH, and conductivity. Next, this polymer solution is loaded into a carriage of a nozzle-less electrospinning device. While reference is made herein to the NANOSPIDER™ nozzle-less or needle-less device, it should be understood that other nozzle-less or needle-less electrospinning devices can be adapted in the manner described herein by one of skill in the art to achieve uniformity and high basis weight of nanofiber fabrics as described herein.

The NANOSPIDER™ electrospinning technology involves a carriage that oscillates along a wire with an applied voltage (30 to 60 kV), with the wire passing through a small orifice (0.5-0.8 mm diameter) in the carriage, entraining the spinning solution on the wire. A second wire (or other geometry) is positioned directly above the spinning wire, with a positively biased voltage applied (10 to 40 kV). A nanofiber collecting substrate is positioned between the two charged wires, and fibers are collected on the bottom of the substrate. The substrate can also move perpendicular to the two electrodes, which can convert this electrospinning arrangement into a continuous, rather than batch, process. Various processing parameters can be chosen and adjusted, including substrate type, carriage traveling distance (wire length), carriage speed, wire rewinding speed, collecting electrode type, applied voltage, distance between electrodes, proportion of voltage applied to each electrode, pass speed, and air flow. The substrate speed, number of passes, and run time dictate, in part, how high the basis weight and total fiber area will be.

Technical barriers for large-scale manufacturing of nanofibers by electrospinning include low yield, lack of uniformity, low speed of fabrication, and the limitation of the process to polymer solutions. For example, when using nozzle-less electrospinning, typically the polymer concentration required is 10% or more of the polymer solution, and the fiber diameters are between 80-1500 nm, with a standard deviation of ±30%. Further, when using current devices for nozzle-less electrospinning, the nanofibers produced have only +15% usable fiber width in the cross direction (CD), there is only a +30% material yield of total mass within 90% maximum basis weight (BW), and uniformity is inconsistent. These values are low and not practical economically for large scale manufacturing of nanofibers, and are unsuitable for biomedical applications.

Accordingly, as described herein, novel compositions, devices, and methods are provided to increase nanofiber fabric yields, basis weights, and uniformity for scalable manufacturing of nanofibers.

### Nozzle-less Electrospinning Devices

Provided herein are nozzle-less electrospinning devices for producing uniform high basis weight, non-woven, polymer nanofiber fabric compositions. Such devices comprise, in part, insulating materials that are used to shield or cover various parts of the nozzle-less electrospinning device, resulting in increased uniformity and high basis weights of electrospun nanofiber fabrics. As understood by one of ordinary skill in the art, a “nozzle-free” or “nozzle-less” electrospinning device refers to any device, apparatus, or machine that can be used to electrospin nanofiber material or fabric in which the polymer solution being electrospun is not fed to a spinneret with nozzles.

As noted above, the NANOSPIDER™ electrospinning technology involves a carriage that oscillates along a wire (referred to herein as the “electrospinning wire” or “electrospinning electrode” or simply, the “spinning wire” or “spinning electrode”) with an applied voltage (30 to 60 kV), with the wire passing through a small orifice (0.5-0.8 mm diameter) in the carriage, entraining the spinning solution on the wire. A second wire (or other electrode geometry, referred to herein as the “collecting wire” or “collecting electrode”) is positioned directly above the electrospinning wire, with a negatively biased voltage applied (−10 to −40 kV). A nanofiber collecting substrate is positioned between the two charged wires, and fibers are collected on the bottom of the substrate. The substrate can also move perpendicular to the two electrodes, which can convert this electrospinning arrangement into a continuous, rather than batch, process. With an arrangement such as this, in which the carriage oscillates a given distance in the “cross-direction” or “carriage-direction” (CD) and the substrate is moved perpendicular to the CD (the direction referred to herein as the “machine direction” (MD)) between the electrospinning electrode and the collecting electrode by way of rollers, the width of the nanospun fiber mat produced will depend upon the measure of the CD dimension, and the length of the nanospun fiber mat produced will depend upon the motion of the substrate during the production run in the MD dimension.

Existing nozzle-less electrospinning devices can only achieve uniformity, as the term is defined herein, at high basis weight, as that term is used herein, over a relatively small portion of the CD dimension, i.e., about 15% of the CD dimension. The improvements described herein can permit the production of uniform high basis weight nanofiber polymer mat or fabric over a significantly wider proportion of the CD dimension, i.e., at least 20%, at least 25%, at least 30%, at least 35%, at least 40%, at least 45%, at least 50%, at least 55%, at least 60%, at least 65%, or at least 70% or more. That is, the improvements described herein permit the production of a uniform, high basis weight nanofiber polymer fabric composition that is wider in the CD dimension than is possible to produce with existing technology. The NANOSPIDER™ device is presently available in 0.5 m, 1.0 m and 1.6 m CD widths. Using these CD sizes as a guide, the 0.5 m device, or its equivalent, can presently produce a high basis weight nanofiber polymer fabric of 10-15 cm in the CD dimension that is uniform as the term is used herein. The improvements described herein can increase that uniform, high basis weight nanofiber polymer fabric to as much as 40 cm in the CD dimension on the same machine. That is, using the improvements described herein, one can produce a high basis weight nanofiber polymer fabric that is uniform, as that term is used herein, over at least 16 cm, at least 18 cm, at least 20 cm, at least 22 cm, at least 24 cm, at least 26 cm, at least 28 cm, at least 30 cm, at least 32 cm, at least 34 cm, at least 36 cm, at least 38 cm or even 40 cm in the CD dimension on a 0.5 m NANOSPIDER™ device or its equivalent. Given the use of long rolls of substrate, such uniform, high basis weight nanofiber polymer fabrics made on a 0.5 m NANOSPIDER™ device or its equivalent using the improvements and methods described herein can be at least 100 cm, at least 200 cm, at least 300 cm, at least 400 cm, at least 500 cm or more in length in the MD dimension, including, for example, at least 600 cm, at least 700 cm, at least 800 cm, at least 900 cm or even 1000 cm in the MD dimension.

A 1.6 m NANOSPIDER™ device or its equivalent can presently produce a high basis weight nanofiber polymer fabric of approximately 24 cm in the CD dimension that is uniform as that term is defined herein. With the improvements described herein, uniformity at high basis weight can be achieved for a nanofiber polymer fabric or mat up to 1.4 m wide in the CD dimension. Thus, using the improvements described herein on a 1.6 m device or its equivalent, one can produce a high basis weight nanofiber polymer fabric that is uniform, as that term is used herein, over at least 25 cm, at least 30 cm, at least 35 cm, at least 40 cm, at least 50 cm, at least 55 cm, at least 60 cm, at least 65 cm, at least 70 cm, at least 75 cm, at least 80 cm, at least 85 cm, at least 90 cm, at least 95 cm, at least 100 cm, at least 105 cm, at least 110 cm, at least 115 cm, at least 120 cm, at least 125 cm, at least 130 cm, at least 135 cm or even 140 cm. Thus, in each instance, the proportion of the CD dimension of a high basis weight nanofiber polymer fabric that is uniform is increased significantly relative to the proportion in the CD dimension without the improvements described herein. The length of such a fiber mat or fabric in the MD dimension is determined by the length of substrate drawn between the electrospinning electrodes during a given run or amount of time. Where a machine can hold a roll of substrate many meters long (e.g., 500 m or more, depending upon the exact machine and the substrate), it is possible to generate high basis weight nanofiber polymer fabrics meters long in the MD dimension. The improvements described herein permit one to increase the proportion, and thereby the overall size, of the fabric that is uniform in the CD dimension. The improvements described herein thus provide for uniform high basis weight nanofiber polymer fabrics that are at least 25 cm wide in the CD dimension, including, for example, uniform high basis weight nanofiber polymer fabrics that are at least 30 cm wide, at least 35 cm wide, at least 40 cm wide, at least 50 cm wide, at least 55 cm wide, at least 60 cm wide, at least 65 cm wide, at least 70 cm wide, at least 75 cm wide, at least 80 cm wide, at least 85 cm wide, at least 90 cm wide, at least 95 cm wide, at least 100 cm wide, at least 105 cm wide, at least 110 cm wide, at least 115 cm wide, at least 120 cm wide, at least 125 cm wide, at least 130 cm wide, at least 135 cm wide or even 140 cm wide in the CD dimension. Given the use of long rolls of substrate, such fabrics can be, for example, at least 100 cm, at least 200 cm, at least 300 cm, at least 400 cm, at least 500 cm or more in length in the MD dimension, including, for example, at least 600 cm, at least 700 cm, at least 800 cm, at least 900 cm or even 1000 cm in the MD dimension. Additional details regarding high basis weight nanofiber polymer fabric compositions that can be produced using the methods, devices and improvements described herein are provided below in the section headed “Nanofiber Fabric Compositions and Methods Thereof”

Accordingly, in some aspects, provided herein are nozzle-less electrospinning devices comprising:
an electrospinning electrode and a collecting electrode comprising connections for a DC power supply, the electrospinning electrode and the collecting electrode spaced apart and establishing an electric field between the electrospinning electrode and the collecting electrode when DC power is supplied;
the collecting electrode comprising a first end and a second end;
the electrospinning electrode comprising a continuously fed or static charged electrode member partially submerged in or carrying an entrained polymer solution to permit electrospinning of fibers of the polymer towards the collecting electrode;
a substrate located between the electrospinning electrode and the collecting electrode such that electrospun polymer fibers become deposited on the substrate when the device is in use;
a first insulating material member encircling the collecting electrode and extending along the collecting electrode from the first end of the collecting electrode towards the second end of the collecting electrode such that a portion of the electrode is covered by the insulating material member and a gap of exposed collecting electrode is formed extending from an end of the insulating material member towards the second end of the collecting electrode;
wherein the first insulating material member increases the uniform area of an electrospun polymer mat deposited on the substrate relative to the uniform area of a polymer mat deposited in the absence of the insulating material member.

As used herein, an “electrospinning electrode” and a “collecting electrode” each comprise an electrically conductive surface, e.g., a conductive metal, such that, when each electrode is connected to a direct current (DC) source, they become electrically charged such that there is a sufficient difference of electric potentials or voltage difference between the two electrically conductive surfaces to induce an electric field strong enough to overcome the surface tension of a given polymer solution. Typically, the electrospinning electrode is connected to a high voltage, DC source and the collecting electrode is connected to the opposite pole of the high voltage DC source or is grounded, such that when the high voltage supply is provided, the polymer nanofibers are drawn from the electrospinning electrode in the direction of the collecting electrode. Typically, the voltage required is at least 10 kV, at least 20 kV, at least 30 kV, at least 40 kV, at least 50 kV, at least 60 kV, at least 70 kV, at least 80 kV, at least 90 kV, at least 100 kV, at least 110 kV, at least 120 kV, or more.

The electrospinning electrode and collecting electrode are typically configured to be parallel or substantially parallel to each other. As used herein, “substantially parallel” refers to two objects, such as two electrodes, that have the same, or approximately the same, distance between them along their entire lengths. In some embodiments of the nozzle-less electrospinning devices described herein, the collecting electrode is substantially parallel to the electrospinning electrode.

As used herein, “spaced apart,” when applied to an electrospinning electrode and collecting electrode refers to a distance between the electrodes sufficient to permit electrospinning of polymer from the electrospinning electrode toward the collecting electrode when DC power is applied to the electrodes. The distance should be sufficient to allow for evaporation and whipping of the nanofiber strands in Taylor cones from individual spinning locations, to permit nanofibers to be deposited upon a substrate situated between the two electrodes. In some embodiments, the electrospinning electrode is separated from the collecting electrode by at least 5 cm, by at least 6 cm, by at least 7 cm, by at least 8 cm, by at least 9 cm, by at least 10 cm, by at least 11 cm, by at least 12 cm, by at least 13 cm, by at least 14 cm, by at least 15 cm, by at least 16 cm, by at least 17 cm, by at least 18 cm, by at least 19 cm, by at least 20 cm, by at least 21 cm, by at least 22 cm, by at least 23 cm, by at least 24 cm, by at least 25 cm, by at least 26 cm, by at least 27 cm, by at least 28 cm, by at least 29 cm or more. In some embodiments, the electrospinning electrode is separated from the collecting electrode by at least 30 cm, by at least 40 cm, by at least 50 cm, by at least 60 cm, by at least 70 cm, by at least 80 cm, by at least 90 cm, by at least 100 cm, by at least 110 cm, by at least 120 cm, by at least 130 cm, by at least 140 cm, by at least 150 cm, by at least 160 cm, by at least 170 cm, by at least 180 cm, by at least 190 cm, by at least 200 cm, by at least 210 cm, by at least 220 cm and usually not more than about 250 cm, typically between 100 and 200 cm.

In regard to an electrode member, e.g., the electrospinning electrode, being “partially submerged” or “partially exposed” in some embodiments, the term refers to the situation in which an electrode is stretched substantially parallel to the surface of a polymer solution and placed in contact with the solution such that a lower surface or portion of the electrode is in contact with the polymer solution and an upper surface of the electrode is above the plane of the surface of the solution. Surface tension of the polymer solution, alone or in conjunction with the electrode, causes a film of polymer solution to cover the upper surface of the electrode to permit electrospinning of the polymer when a DC current is applied to the electrospinning and collecting electrodes. Where the electrospinning electrode is alternatively configured as a wire passing through a small orifice to continuously supply entrained polymer solution, the electrode is not “partially submerged” in the polymer solution and can be referred to as partially exposed to the polymer solution.

Where “a portion” of the collecting electrode is covered or encircled by an insulating material member, a “portion” will include at least 5% of the length of exposed electrode, but generally can be less than or equal to 25%, less than or equal to 20%, or less than or equal to 15%, less than or equal to 10% of the exposed surface length of the collecting electrode.

As used herein, a “substrate” refers to any suitable material upon which electrospun polymer nanofibers can be deposited by electrospinning Preferred substrates can be supplied in a sheet or roll form. Such substrates can include natural and synthetic substrates such as paper or waxed paper, spun-bonded fabrics, non-woven fabrics of synthetic fiber, non-wovens made from blends of cellulose materials, synthetics and glass fibers, non-woven and woven glass fabrics, plastic materials, and foils, such aluminum foil or copper foil. Ideally, the substrate used will permit deposition of the nanospun fabric on the substrate while also permitting removal of the fabric from the substrate, e.g., by peeling off the fabric. The non-adhesive or non-stick property of waxed paper provides advantages in this regard, in some embodiments.

Accordingly, in some embodiments of the nozzle-less electrospinning devices described herein, the substrate is selected from waxed paper, parchment paper, silicone coated paper, QUILON coated paper, glassine paper, polypropylene spunbond, cellulosic paper, aluminum foil, copper foil and polytetra fluoroethylene (Teflon) sheeting.

In some embodiments of the nozzle-less or needle-less electrospinning devices described herein, the substrate is substantially planar. As used herein, “substantially planar,” when used in reference to a substrate, refers to a material which forms a sheet stretched horizontally between the electrospinning electrode and collecting electrode. While the weight and composition of the substrate can permit some degree of sagging between the points at which it is suspended (in some embodiments, the substrate is stretched between rollers), the overall configuration of the substrate between the suspension points is a plane substantially parallel to the lines defined by the electrospinning and collecting electrodes, respectively.

In some embodiments of the nozzle-less electrospinning devices described herein, the substrate is configured to move perpendicular to the direction of the collecting electrode when the device is in use.

In some embodiments of the nozzle-less electrospinning devices described herein, the substrate is arranged on one or more rollers to permit the substrate to move perpendicular to the direction of the collecting electrode when the device is in use.

In some embodiments of the nozzle-less electrospinning devices described herein, the device further comprises a second insulating material element encircling the collecting electrode and extending along the collecting electrode from the second end of the collecting electrode toward the first end of the collecting electrode such that a portion of the collecting electrode is covered by the second insulating material member and the gap of exposed collecting electrode extends between the first and second insulating material elements.

Insulating materials useful in various aspects and embodiments of the nozzle-free electrospinning devices described herein include any material having a dielectric constant or relative permittivity of at least 1.2 that can be configured or designed to encircle and extend along a portion of the nozzle-free electrospinning device, such as the collecting electrode, or to block or shield a portion of the nozzle-free electrospinning device, such as the collecting electrode. As known to one of ordinary skill in the art, the dielectric constant or relative permittivity is the ratio of the capacitance of a capacitor using a given material as a dielectric, compared to a similar capacitor that has vacuum as its dielectric. If a material with a high dielectric constant is placed in an electric field, such as the electric filed of a nozzle-free electrospinning device, the magnitude of that field will be measurably reduced within the volume of the material with a high dielectric constant. Thus, in some embodiments of the aspects described herein, an insulating material for use in the nozzle-free electrospinning devices described herein has a dielectric constant of at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.1, at least 3.2, at least 3.3, at least 3.4, at least 3.5, at least 3.6, at least 3.7, at least 3.8, at least 3.9, at least 4.0, at least 5.0, at least 6.0, at least 7.0, at least 8.0, at least 9.0, at least 10.0, or more, and can be configured or designed to encircle and extend along a portion of the collecting electrode. In other embodiments, the insulating material can provide shielding or shaping of the electric field in other configurations, as described herein.

Accordingly, in some embodiments of the nozzle-less electrospinning devices described herein, the first insulating material member has a dielectric constant of at least 1.2. In some embodiments of the nozzle-less electrospinning devices described herein, a second insulating material member has a dielectric constant of at least 1.2. In some embodiments of the nozzle-less electrospinning devices described herein, such first and second insulating material members comprise the same insulating material. The dielectric constants of various materials are provided, for example, in Table 1, below.

Dielectric Constants

| Material | Dielectric Constant | Dielectric Loss Tangent |
|---|---|---|
| Air | 1.0 | — |
| Butyl Rubber (BR) | 2.35 | 0.001-0.0009 |
| Cement | 2.0 | — |
| Cotton | 1.3 | — |
| Glass | 3.7-10 | — |
| Polyethylene (PE) | 2.3-2.7 | 0.0002-0.00031 |
| Polystyrene (PS) | 2.5-2.9 | 0.0001-0.00033 |
| Teflon | 2.1 | 0.0005-0.00028 |
| Waxed Paper | 3.7 | — |

Polyethylene is used for wire insulation and intentionally foamed to reduce its dielectric constant and loss factor (Dielectric Properties of Polyethylene foams at Medium and High Frequencies; Strååt et al.)

Non-limiting examples of insulating materials for use in some embodiments of the nozzle-free electrospinning devices described herein (and, for some, their dielectric constants at room temperature under 1 kHz in parentheses) include rubber (7), glass (3.7-10), cotton, perlite, charcoal, wood, fiberglass, fiberglass insulation, polyethylene (2.25), high density polyethylene, low density polyethylene, polypropylene (2.2-2.36), polystyrene (2.4-2.7) and foamed versions of polyethylene, polypropylene and polystyrene, polyvinyl alcohol and polytetra fluoroethylene or Teflon (2.1).

In some embodiments of the nozzle-less electrospinning devices described herein, the first insulating material is selected from rubber, glass, cotton, perlite, charcoal, wood, fiberglass, fiberglass insulation, polyethylene, high density polyethylene, low density polyethylene, polypropylene, polystyrene and foamed versions of polyethylene, polypropylene and polystyrene, polyvinyl alcohol and polytetra fluoroethylene.

In some embodiments of the nozzle-less electrospinning devices described herein, the first insulating material, i.e., the insulating material encircling a portion of the collecting electrode, is butyl rubber.

In some embodiments of the nozzle-less electrospinning devices including insulating material encircling a portion of the collecting electrode, the modification permits the generation of uniform high basis weight nanofiber polymer fabrics that are at least 25 cm or more wide in the CD dimension, including, for example, uniform high basis weight nanofiber polymer fabrics that are at least 30 cm wide, at least 35 cm wide, at least 40 cm wide, at least 50 cm wide, at least at least 55 cm wide, at least 60 cm wide, at least 70 cm wide, at least 80 cm wide, at least 90 cm wide, at least 100 cm wide, at least 110 cm wide or more in the CD dimension. Such improved dimensions can be achieved, for example, on a 1.6 m NANOSPIDER™ device or its equivalent using the insulating material modification on the collecting electrode as described herein. Given the use of long rolls of substrate, such fabrics can be, for example, at least 100 cm, at least 200 cm, at least 300 cm, at least 400 cm, at least 500 cm or more in length in the MD dimension, including, for example, at least 600 cm, at least 700 cm, at least 800 cm, at least 900 cm or even 1000 cm in the MD dimension. Thus, this improvement provides uniform high basis weight nanofiber polymer fabrics at least 25 cm by 100 cm in size, at least 30 cm by 100 cm, at least 35 cm by 100 cm, at least 40 cm by 100 cm, at least 50 cm by 100 cm, at least 55 cm by 100 cm, at least 60 cm by 100 cm, at least 70 cm by 100 cm, at least 80 cm by 100 cm, at least 90 cm by 100 cm, at least 100 cm by 100 cm, at least 110 cm by 100 cm or more, including, for example, at least 25 or 30 cm by at least 1000 cm.

In some embodiments of the nozzle-less electrospinning devices described herein, the electrospinning electrode comprises a charged surface from which fibers are electrospun, and wherein the length of the gap of exposed collecting electrode is aligned with and substantially the same length or less than the charged surface of the electrospinning electrode from which fibers are electrospun.

In some aspects, provided herein are nozzle-less electrospinning devices comprising:
an electrospinning electrode and a collecting electrode comprising connections for a DC power supply, the electrospinning electrode and the collecting electrode spaced apart and establishing an electric field between the electrospinning electrode and the collecting electrode when DC power is supplied;
the collecting electrode comprising a first end and a second end;
the electrospinning electrode comprising a rotating continuously fed or static charged electrode member partially submerged in or carrying an entrained polymer solution to permit electrospinning fibers of the polymer towards the collecting electrode;
a substrate located between the electrospinning electrode and the collecting electrode such that electrospun polymer fibers become deposited on the substantially planar substrate when in use;
a first shield comprised of a first insulating material member situated between the substrate and the collecting electrode and extending from the first end of the collecting electrode towards the second end of the collecting electrode such that a portion of the electric field is shielded by the first insulating material member and a gap of unshielded collecting electrode is formed extending from an end of the first insulating material member towards the second end of the collecting electrode;
wherein the first shield increases the uniform area of an electrospun polymer mat deposited on the substrate relative to the uniform area of a polymer mat deposited in the absence of the first shield.

In regard to the electrospinning devices described herein, a “shield” refers to an element comprising any of the insulating materials described herein having a dielectric constant of at least 1.2 that is placed in the electric field between electrospinning and collecting electrodes to influence the direction of electrospinning In certain embodiments, one or more shields modulate the spread of the electric field generated between the electrospinning electrode and the collecting electrode, thereby increasing the uniform area of an electrospun polymer mat deposited on the substrate, when compared to the same electrospinning device in the absence of the shield.

In some embodiments of these nozzle-less electrospinning devices, the device further comprises a second shield comprised of a second insulating material member situated between the substrate and the collecting electrode and extending from the second end of the collecting electrode towards the first end of the collecting electrode such that a portion of the electric field is shielded by the second insulating material member and the gap of unshielded collecting electrode extends between an end of the first insulating material member and an end of the second insulating material member, wherein the second shield further increases the uniform area of an electrospun polymer mat deposited on the substrate relative to the uniform area of a polymer mat deposited in the absence of the second shield.

The inclusion of the first shield, alone, or preferably, together with the second shield, provides an increase in uniform area achievable with a free-surface electrospinning device relative to such device, e.g., a NANOSPIDER™ device or its equivalent, lacking such shielding. In such embodiments of the nozzle-less electrospinning devices including such shielding, the modification permits the generation of uniform high basis weight nanofiber polymer fabrics that are at least 25 cm or more wide in the CD dimension, including, for example, uniform high basis weight nanofiber polymer fabrics that are at least 30 cm wide, at least 35 cm wide, at least 40 cm wide, at least 45 cm wide, at least 50 cm wide, at least 55 cm wide, at least 60 cm wide, at least 70 cm wide, at least 80 cm wide, at least 90 cm wide, at least 100 cm wide, at least 110 cm wide or more in the CD dimension. Such improved dimensions can be achieved, for example, on a 1.6 m NANOSPIDER™ device or its equivalent using such shielding modification as described herein. The uniformity in the CD dimension using, for example, a 0.5 m NANOSPIDER™ device or its equivalent is improved from about 15 cm with no modification to at least 16 cm, at least 17 cm, at least 18 cm, at least 19 cm, at least 20 cm, at least 25 cm, at least 30 cm, at least 35 cm, up to about 40 cm with the shielding. Given the use of long rolls of substrate, such fabrics can be, for example, at least 100 cm, at least 200 cm, at least 300 cm, at least 400 cm, at least 500 cm or more in length in the MD dimension, including, for example, at least 600 cm, at least 700 cm, at least 800 cm, at least 900 cm or even 1000 cm in the MD dimension. Thus, this improvement provides uniform high basis weight nanofiber polymer fabrics at least 25 cm by 100 cm in size, at least 30 cm by 100 cm, at least 35 cm by 100 cm, at least 40 cm by 100 cm, at least 45 cm by 100 cm, at least 50 cm by 100 cm, at least 55 cm by 100 cm, at least 60 cm by 100 cm, at least 70 cm by 100 cm, at least 80 cm by 100 cm, at least 90 cm by 100 cm, at least 100 cm by 100 cm, at least 110 cm by 100 cm or more, including, for example, at least 25 or 30 cm by at least 1000 cm.

Also provided for use in the electrospinning devices described herein are one or more “collimating shields.” A collimating shield is a specific type of shield as described herein that is placed between the electrospinning electrode and the substrate, and can be comprised of any of the insulating materials described herein. As used herein, a “collimating shield” is configured and used to minimize the spread of the electrical field lines between the electrospinning electrode and the collecting electrode, thereby making the electrical field lines between them more parallel. By making the electrical field lines more parallel, as shown herein, there is an increase in the uniform area of an electrospun nanofiber polymer mat deposited on the substrate of the electrospinning device, when compared to the uniform area deposited in the absence of the collimating shield.

In some embodiments of these nozzle-less electrospinning devices, the device further comprises a first collimating shield comprised of a third insulating material member, the first collimating shield supported and situated adjacent to the substrate and between the substrate and the electrospinning electrode, the first collimating shield extending substantially perpendicular to the collecting electrode, an edge of the first collimating shield facing the gap of unshielded collecting electrode aligned with the end of the first shield insulating material member adjacent the gap of unshielded collecting electrode, wherein the first collimating shield increases the uniform area of an electrospun polymer mat deposited on the substrate relative to the uniform area of a polymer mat deposited in the absence of the collimating shield.

In some embodiments of these nozzle-less electrospinning devices, the device further comprises a second collimating shield comprised of a fourth insulating material member, the second collimating shield supported and situated adjacent to the substrate and between the substrate and the electrospinning electrode, the second collimating shield extending substantially perpendicular to the collecting electrode, an edge of the second collimating shield facing the gap of unshielded collecting electrode aligned with the end of the first shield insulating material member adjacent the gap of unshielded collecting electrode, wherein the second collimating shield increases the uniform area of an electrospun polymer mat deposited on the substrate relative to the uniform area of a polymer mat deposited in the absence of such collimating shield.

In some embodiments of the aspects described herein, where more than one collimating shield is used with a nozzle-less electrospinning device, the collimating shields are made of the same insulating material. In some embodiments of the aspects described herein, where more than one collimating shield is used in the nozzle-less electrospinning devices, the collimating shields are made of different insulating materials. It is specifically contemplated herein that first and/or second collimating shields as described herein can, alone, provide a benefit in uniform fabric area in the absence of the other modifications described herein. In practice, and as demonstrated herein, the collimating shields are most likely to be used with insulating, shielding and electrical contact modifications described herein and, for example, with the process improvements described herein.

The inclusion of the first collimating shield, alone, or preferably, together with the second collimating shield, provides an increase in uniform area achievable with a free-surface electrospinning device relative to such device, such as a NANOSPIDER™ device or its equivalent, lacking such collimating shield(s). In such embodiments of the nozzle-less electrospinning devices including such collimating shielding, the modification permits the generation of uniform high basis weight nanofiber polymer fabrics that are at least 25 cm or more wide in the CD dimension, including, for example, uniform high basis weight nanofiber polymer fabrics that are at least 30 cm wide, at least 35 cm wide, at least 40 cm wide, at least 45 cm wide, at least 50 cm wide, at least 55 cm wide, at least 60 cm wide, at least 70 cm wide, at least 80 cm wide, at least 90 cm wide, at least 100 cm wide, at least 110 cm wide or more in the CD dimension. Such improved dimensions can be achieved, for example, on a 1.6 m NANOSPIDER™ device or its equivalent using such collimating shielding modification as described herein. The uniformity in the CD dimension using, for example, a 0.5 m NANOSPIDER™ device or its equivalent is improved with collimating shielding from about 15 cm with no modification to at least 16 cm, at least 17 cm, at least 18 cm, at least 19 cm, at least 20 cm, at least 25 cm, at least 30 cm, at least 35 cm, up to about 40 cm. Given the use of long rolls of substrate, such fabrics can be, for example, at least 100 cm, at least 200 cm, at least 300 cm, at least 400 cm, at least 500 cm or more in length in the MD dimension, including, for example, at least 600 cm, at least 700 cm, at least 800 cm, at least 900 cm or even 1000 cm in the MD dimension. Thus, this improvement provides uniform high basis weight nanofiber polymer fabrics at least 25 cm by 100 cm, at least 30 cm by 100 cm, at least 35 cm by 100 cm, at least 40 cm by 100 cm, at least 45 cm by 100 cm, at least 50 cm by 100 cm, at least 60 cm by 100 cm, at least 70 cm by 100 cm, at least 80 cm by 100 cm, at least 90 cm by 100 cm, at least 100 cm by 100 cm, at least 110 cm by 100 cm or more, including, for example, at least 25 or 30 cm by at least 1000 cm.

In some embodiments of these nozzle-less electrospinning devices, the device further comprises a first encircling insulating material member encircling the collecting electrode and extending along the collecting electrode from the first end of the collecting electrode towards the second end of the collecting electrode such that a portion of the electrode is covered by the first encircling insulating material member and a gap of exposed collecting electrode is formed extending from an end of the first encircling insulating material member towards the second end of the collecting electrode.

In some embodiments of these nozzle-less electrospinning devices, the device further comprises a second encircling insulating material member encircling the collecting electrode and extending along the collecting electrode from the second end of the collecting electrode towards the first end of the collecting electrode such that a portion of the electrode is covered by the second encircling insulating material member and a gap of exposed collecting electrode is defined extending from an end of the first encircling insulating material member to an end of the second encircling insulating material member.

The inclusion of the first, and preferably the second, encircling insulating material member encircling the collecting electrode, along with the shielding described can further improve the length of uniformity in the CD dimension.

In some embodiments of the nozzle-less electrospinning devices, the first shield comprises an insulating material selected from rubber, e.g., butyl rubber, glass, cotton, perlite, charcoal, wood, fiberglass, fiberglass insulation, polyethylene, high density polyethylene, low density polyethylene, polypropylene, polystyrene and foamed versions of polyethylene, polypropylene and polystyrene, polyvinyl alcohol and polytetra fluoroethylene. In some embodiments of the nozzle-less electrospinning devices, the first shield comprises polyethylene foam.

In some embodiments of the nozzle-less electrospinning devices, the second shield comprises an insulating material selected from rubber, glass, cotton, perlite, charcoal, wood, fiberglass, fiberglass insulation, polyethylene, high density polyethylene, low density polyethylene, polypropylene, polystyrene and foamed versions of polyethylene, polypropylene and polystyrene, polyvinyl alcohol and polytetra fluoroethylene. In some embodiments of the nozzle-less electrospinning devices, the second shield comprises polyethylene foam.

In some embodiments of the nozzle-less electrospinning devices, the collimating shield comprises an insulating material selected from rubber, glass, cotton, perlite, charcoal, wood, fiberglass, fiberglass insulation, polyethylene, high density polyethylene, low density polyethylene, polypropylene, polystyrene and foamed versions of polyethylene, polypropylene and polystyrene, polyvinyl alcohol and polytetra fluoroethylene. In some embodiments of the nozzle-less electrospinning devices, the collimating shield comprises polyethylene foam.

In some embodiments of the nozzle-less electrospinning devices, the encircling, insulating material comprises a material selected from rubber, glass, cotton, perlite, charcoal, wood, fiberglass, fiberglass insulation, polyethylene, high density polyethylene, low density polyethylene, polypropylene, polystyrene and foamed versions of polyethylene, polypropylene and polystyrene, polyvinyl alcohol and polytetra fluoroethylene. In some embodiments of the nozzle-less electrospinning devices, the encircling, insulating material comprises polyethylene foam.

In some embodiments of the nozzle-less electrospinning devices, the substrate is selected from waxed paper, parchment paper, silicone coated paper, QUILON coated paper, glassine paper, polypropylene spunbond, cellulosic paper, aluminum foil, copper foil and polytetra fluoroethylene (TEFLON™) sheeting.

In some embodiments of the nozzle-less electrospinning devices, the substrate is configured to move perpendicular to the direction of the collecting electrode when the device is in use.

In some embodiments of the nozzle-less electrospinning devices, the substrate is arranged on one or more rollers to permit the substrate to move perpendicular to the direction of the collecting electrode when the device is in use.

In some embodiments of the nozzle-less electrospinning devices, the collecting electrode is substantially parallel to the electrospinning electrode.

In some embodiments of the nozzle-less electrospinning devices, the electrospinning electrode comprises a charged surface from which fibers are electrospun, and wherein the length of the gap of exposed collecting electrode is aligned with and substantially the same length as the charged surface of the electrospinning electrode from which fibers are electrospun.

Various embodiments of the technology disclosed are described with reference to the figures in the following.

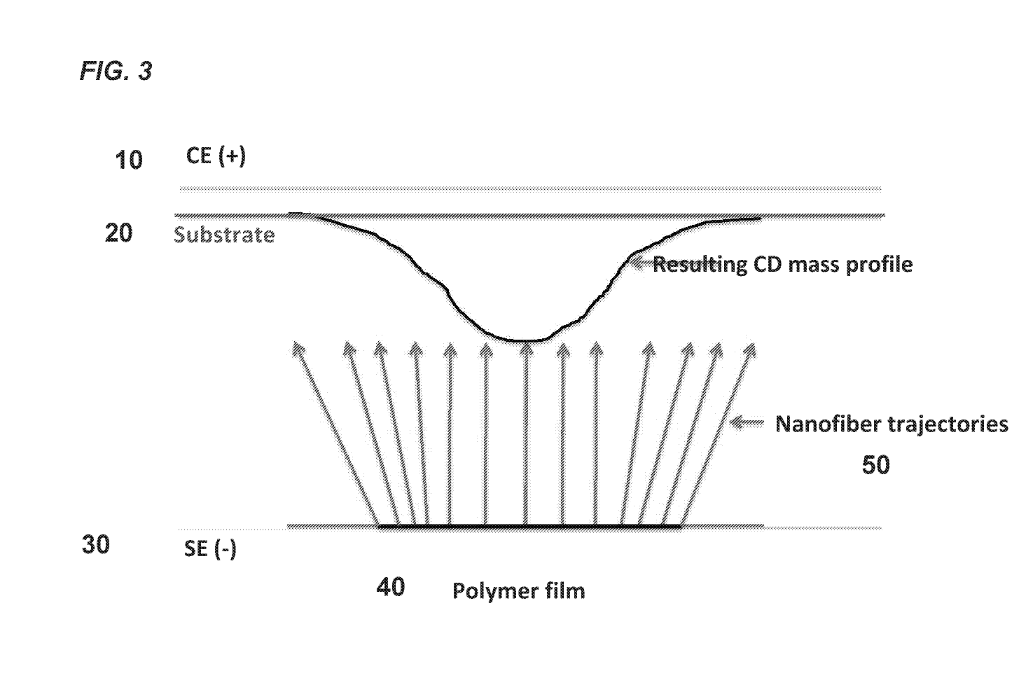

FIG. 3 shows a schematic of an electrospinning apparatus prior to the modifications described herein that permit greater uniformity for electrospun nanofiber fabrics. Electrospinning electrode (SE) 30, collecting electrode, (CE) 10, substrate 20, polymer film 40 on the SE 30 and nanofiber trajectories 50 are shown. In use, nanofibers are ejected from the polymer film 40 on the electrospinning electrode 30 towards the collecting electrode 10 and become deposited on the substrate 20 located between the electrospinning electrode 30 and collecting electrode 10. The standard arrangement shown generally results in fiber deposition with a cross-direction (CD) mass profile having a bell-shaped distribution. The various modifications to this general design described herein flatten and spread the bell-shaped distribution, thereby achieving uniformity over a greater area and minimizing waste, which renders them well-suited for production of nanofiber compositions for the delivery of biologically active agents.

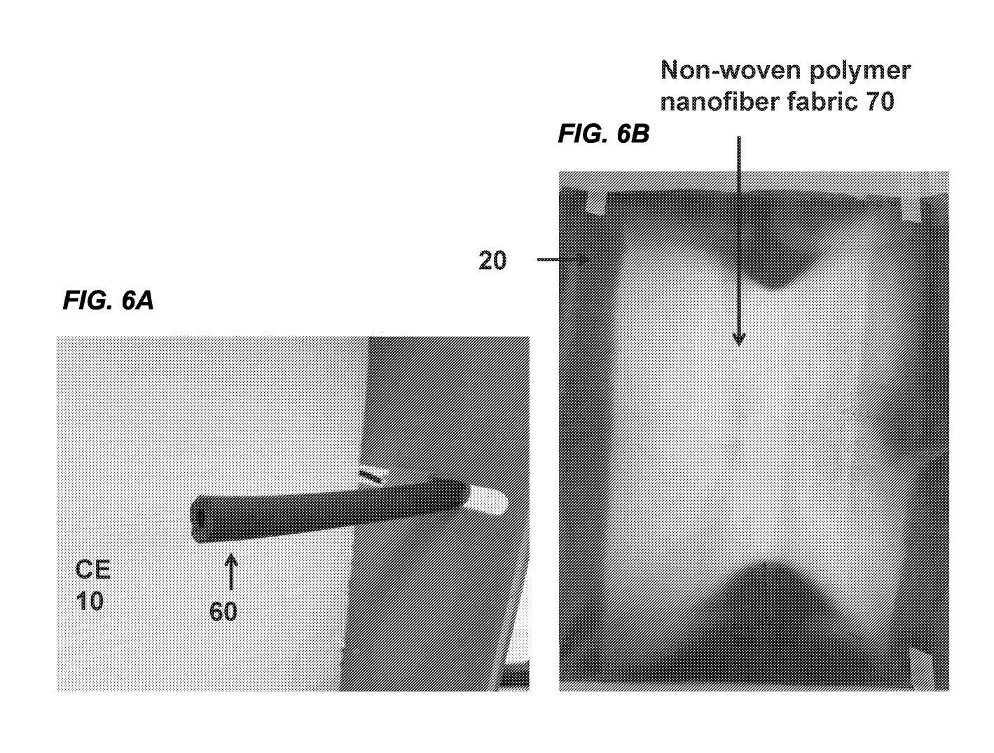

It was found that greater uniformity in the deposited nanofiber fabric could be achieved through use of an insulating material encircling a portion of the collecting electrode. FIG. 6A shows an embodiment in which an insulating material member 60, encircles a portion of the collecting electrode 10. In this embodiment, insulating material element 60 is comprised of butyl rubber. A razor was used to split vacuum tubing and two pieces were placed, one at each end, to create a 22 cm gap of bare collecting electrode. The resulting electrospun, non-woven, nanofiber fabric 70 is shown in FIG. 6B on the waxed paper substrate 20. This modification provides for increased uniformity of a non-woven, nanofiber polymer fabric.

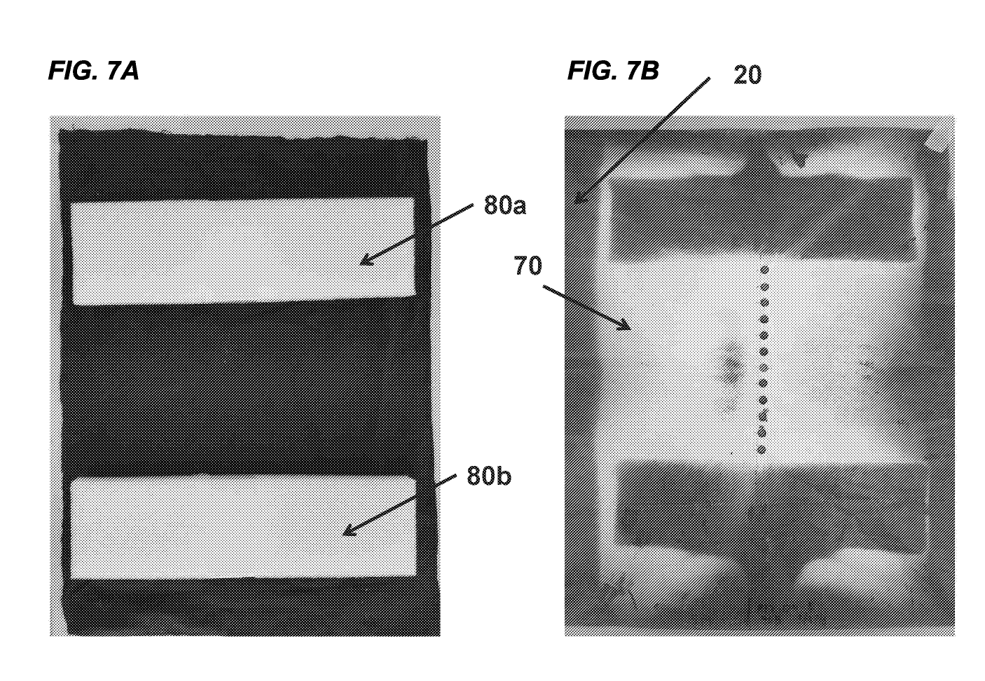

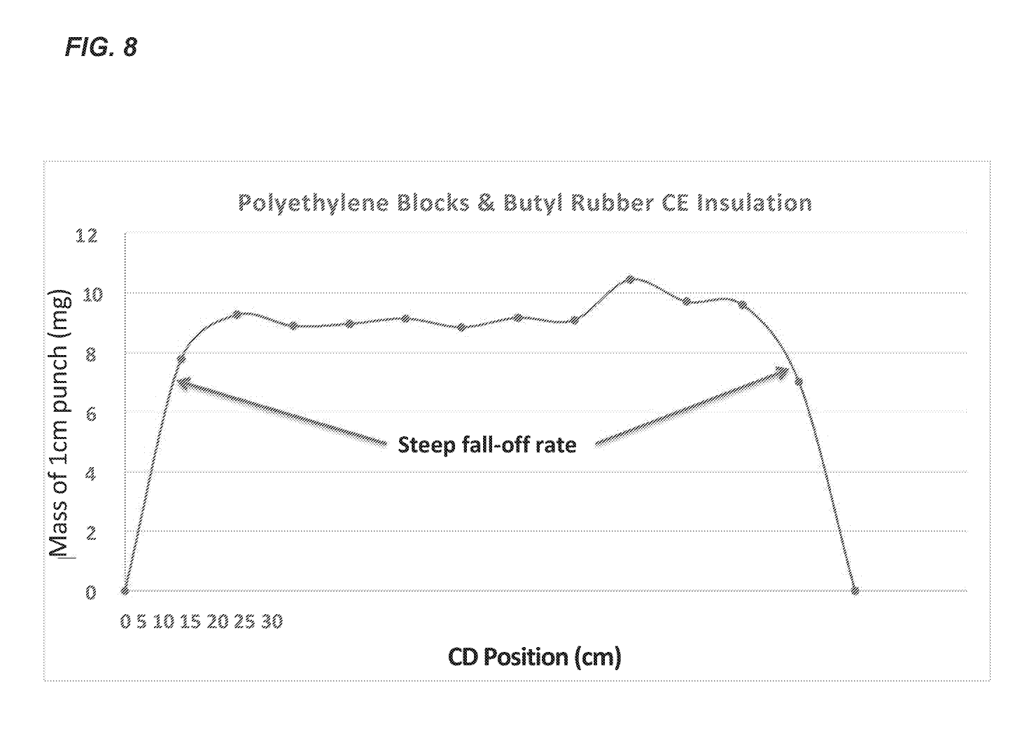

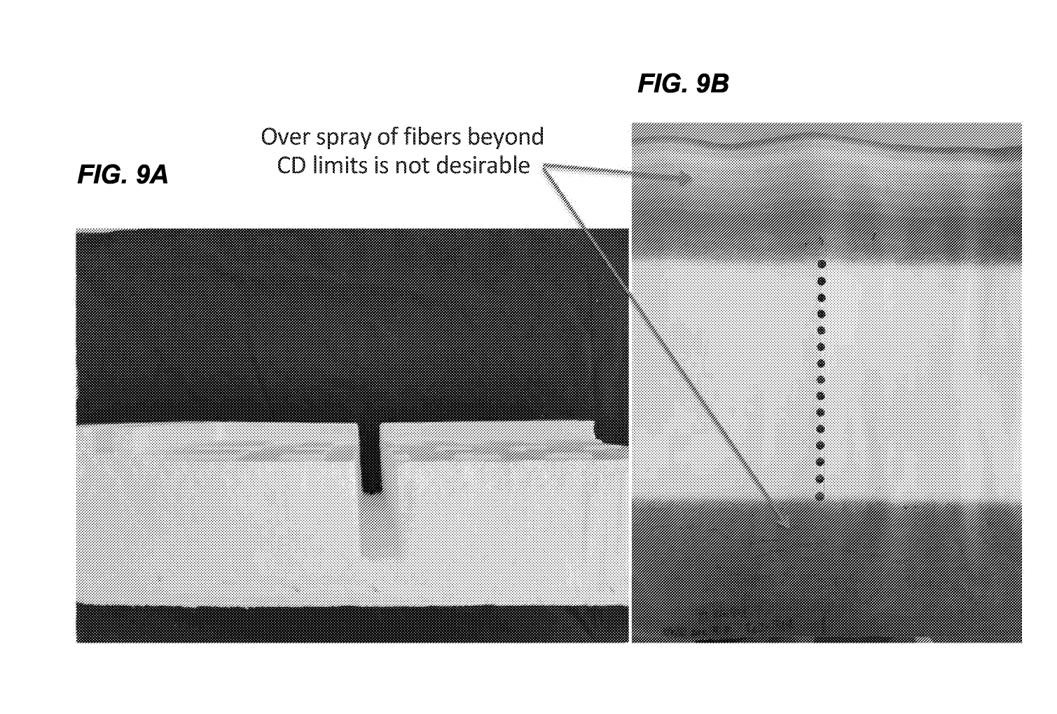

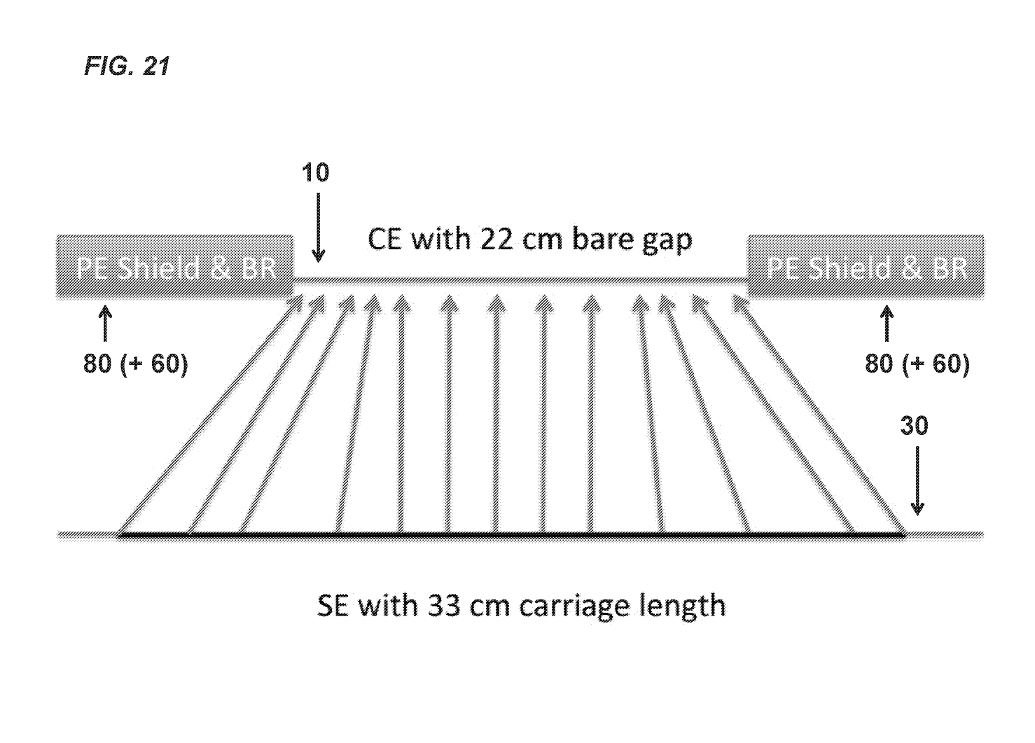

Separately, or in addition to the insulating material member encircling the collecting electrode, it was found that the use of various configurations of shielding further changed the electric field and thereby the pattern of nanofiber deposition. FIG. 7A shows an embodiment in which insulating blocks 80a and 80b (here, polyethylene) placed above the substrate 20, between the substrate 20 and the collecting electrode 10 also increased the area in which nanofibers were deposited uniformly (7B). A schematic view of this general arrangement is shown in FIG. 21, where “PE Shield & BR” indicates polyethylene shielding 80a and 80b, with a butyl rubber insulating material 60a and 60b encircling the collecting electrode 10. FIG. 9A shows an embodiment of this arrangement in which a groove 100 in a block of polyethylene shielding 80 provides clearance for the insulating material 60 encircling the collecting electrode (the butyl rubber insulating material and collecting electrode are not shown in FIG. 9A-9B). In this way, a first shield 80 is combined with an insulating material 60 encircling the collecting electrode 10 to further improve the uniform area of nanofiber deposition, as evident in FIG. 9B and in the graphical representation of mass distribution shown in FIG. 8.

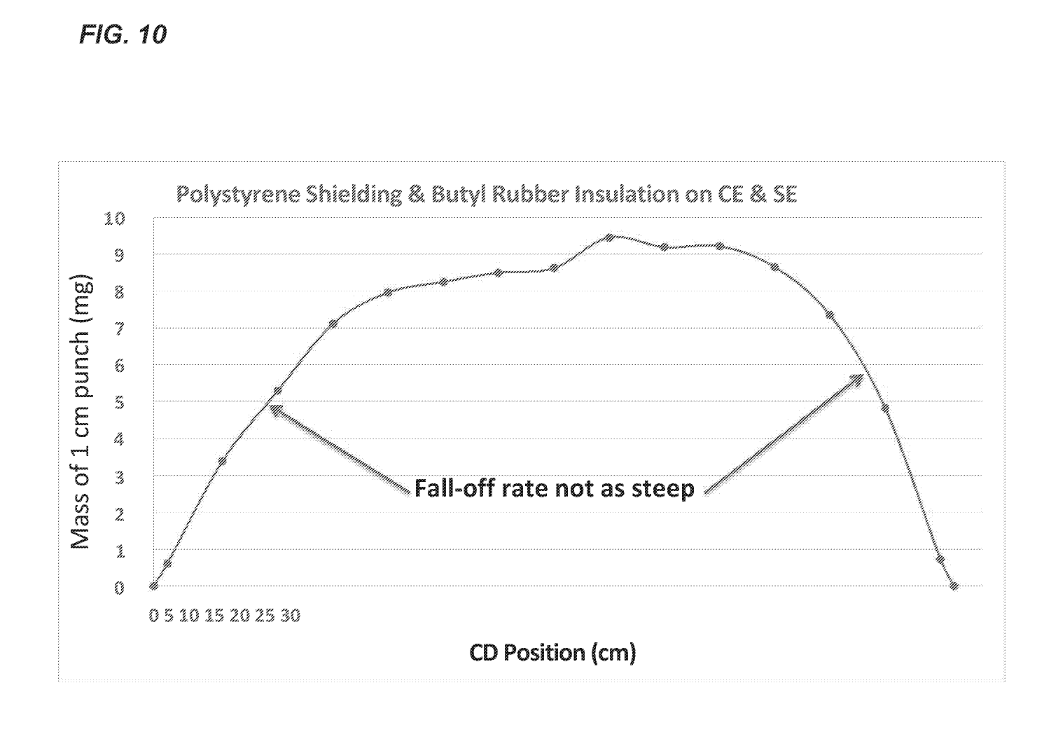

FIG. 10 shows the results using polystyrene shielding and butyl rubber insulation on the collecting electrode and electrospinning electrode. The combination provided an improvement in uniform area relative to no shielding or insulating material use. It was noted that the fall-off rate at the edges was not as steep as achieved with butyl rubber insulating material on the collecting electrode and polyethylene shielding. A steeper fall-off rate helps minimize the non-uniform areas at the edges of the nanofiber fabric, and thereby minimize waste.

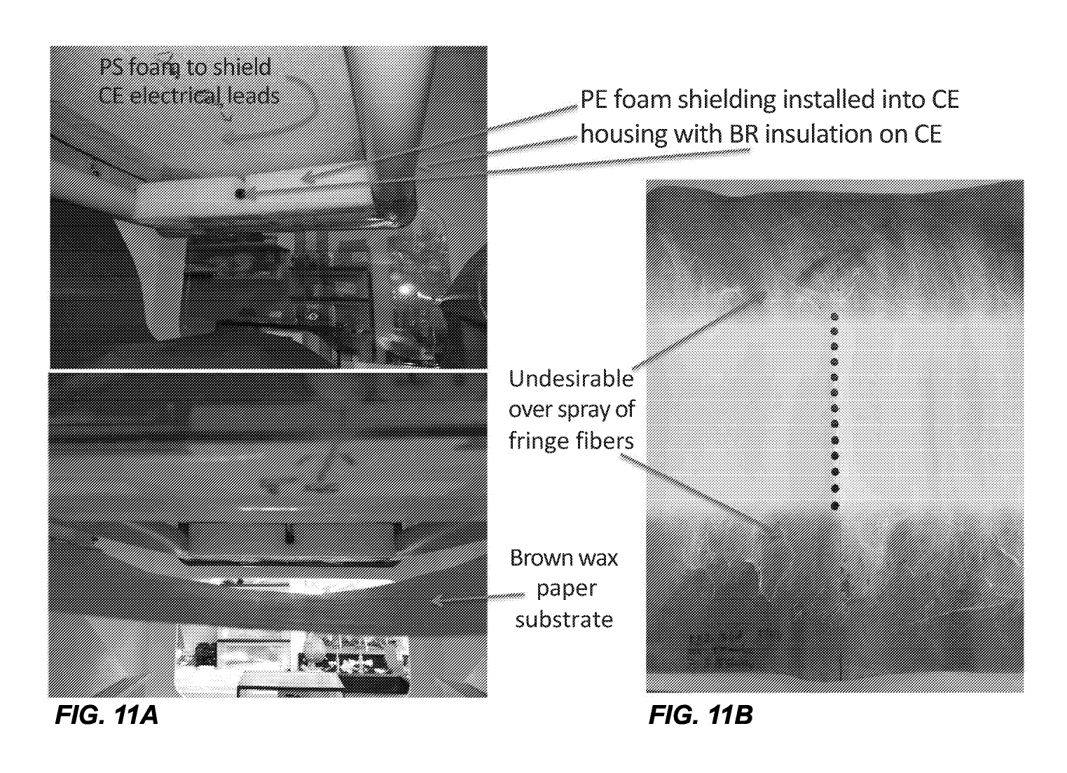

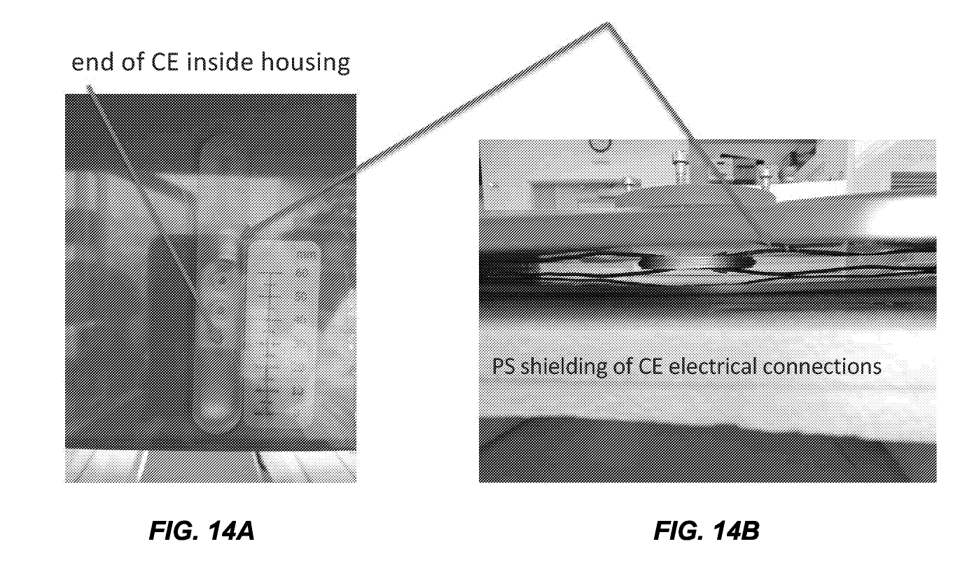

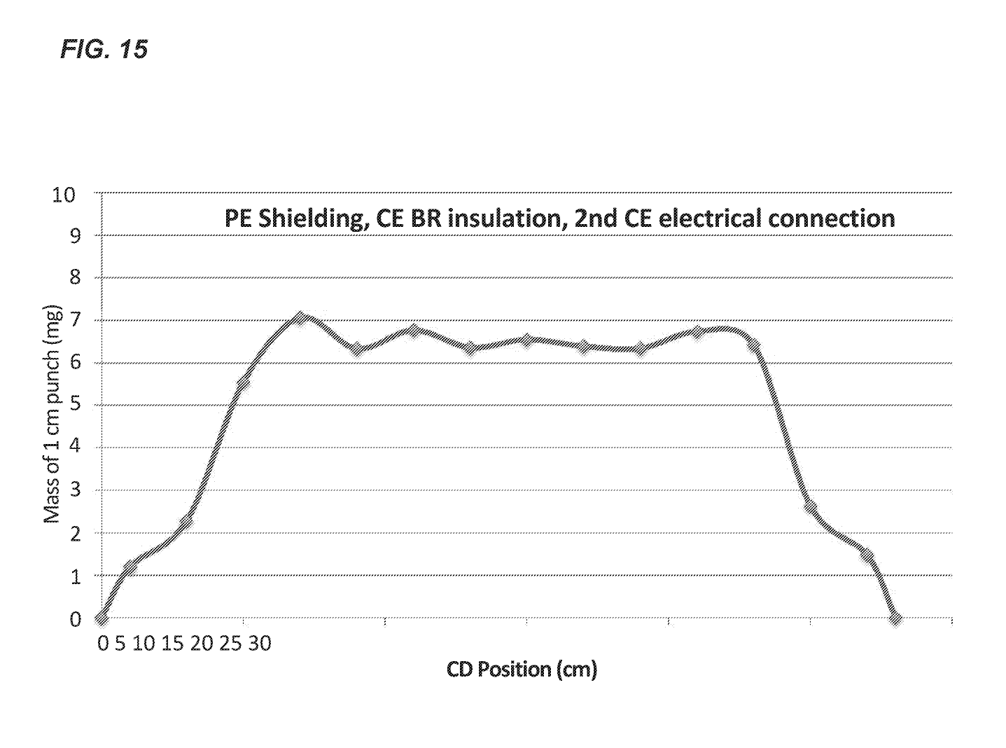

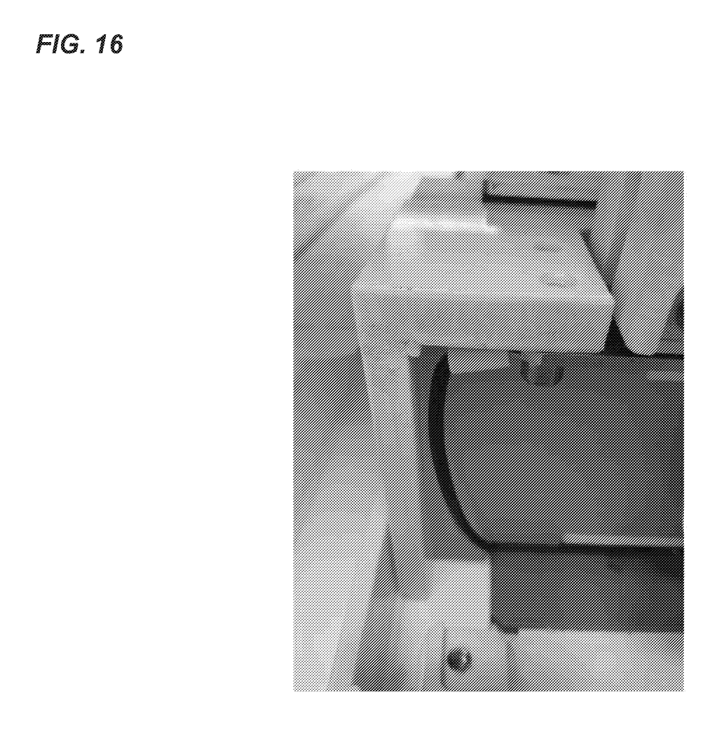

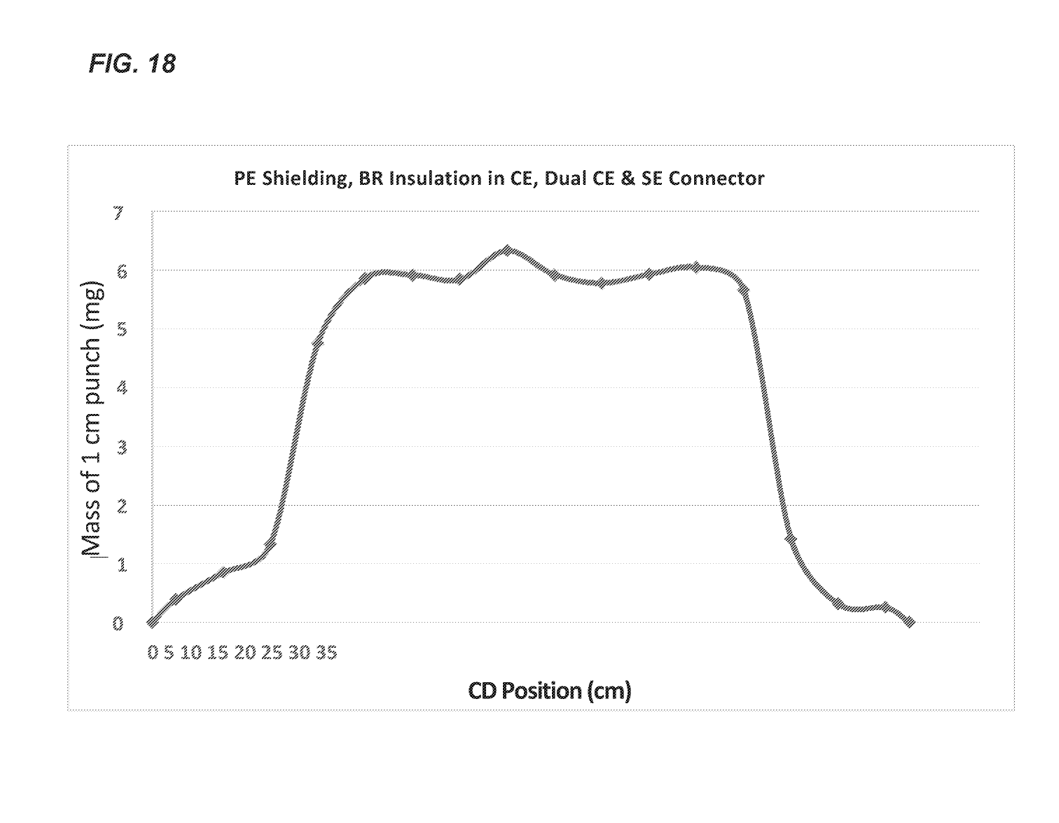

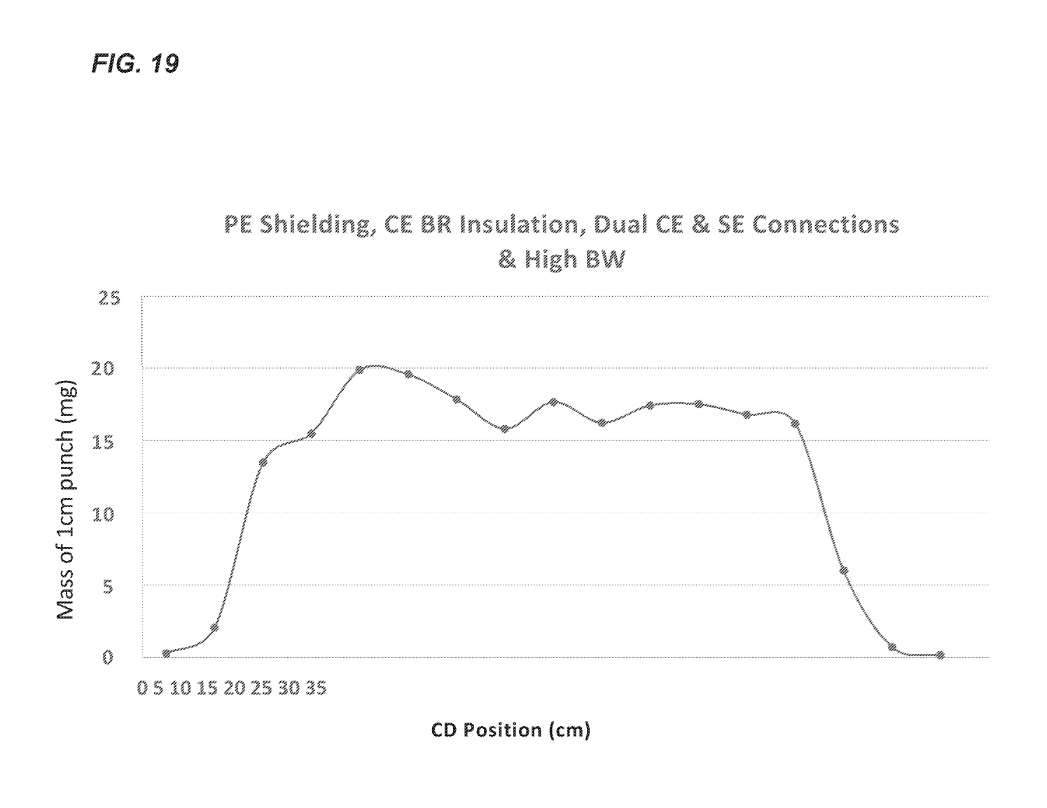

It was also considered whether the electrical connection to the collecting electrode changed the deposition pattern of the nanofiber mat or fabric. FIG. 11A shows how polystyrene foam, placed between the collecting electrode and the collecting electrode electrical leads was used to shield the collecting electrode electrical leads. Another view is shown in FIG. 14B. Results are shown in FIG. 11B. It was also found that providing additional electrical connections to the electrodes, shown in FIG. 16, improved the mass deposition profile. FIG. 15 shows the mass distribution profile when a second collecting electrode electrical connection was added. See also, FIGS. 18 and 19 which show mass deposition profiles using polyethylene shielding, butyl rubber insulation on the collecting electrode, and dual connections on both the spinning electrode and the collecting electrode. In FIG. 18, 15 machine direction passes were performed, while in FIG. 19, 33 machine direction passes were performed, resulting in a roughly 2X increase in mass between FIG. 18 and FIG. 19.

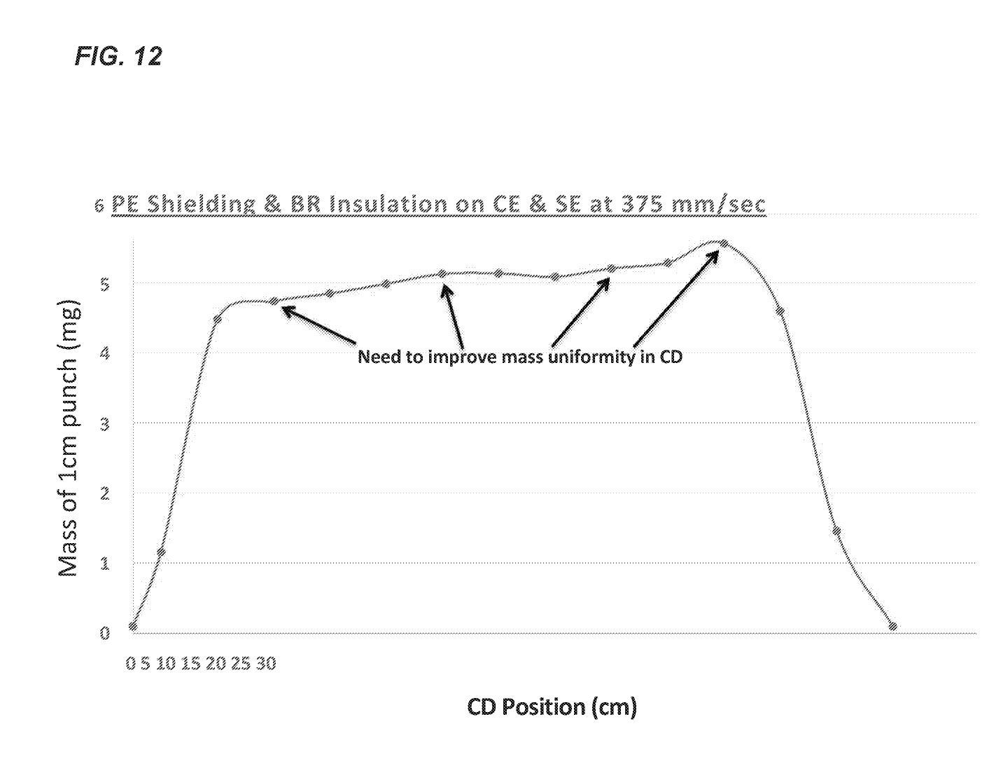

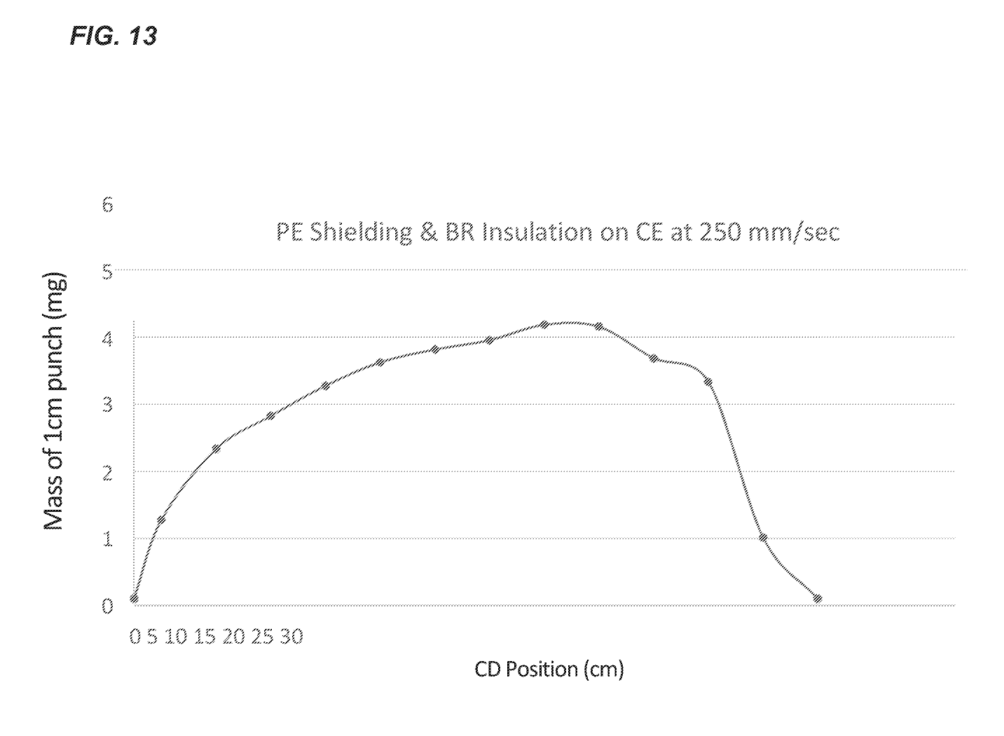

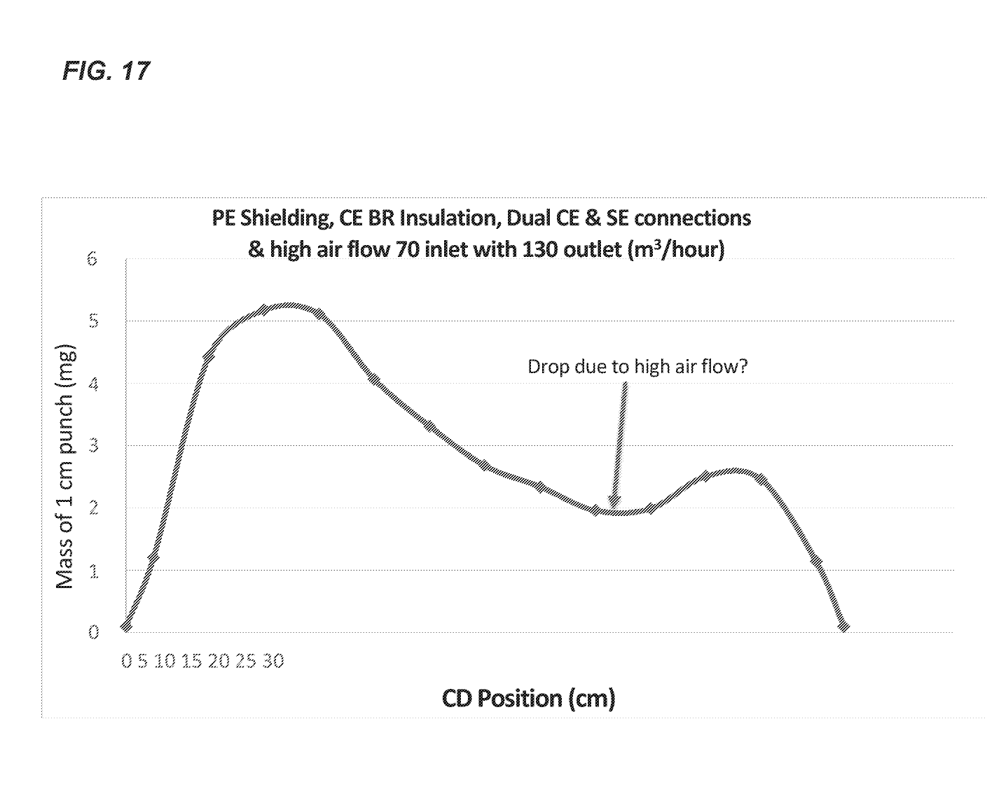

Further improvements in uniformity can be gained by examining different rates of carriage movement. The effects of insulation on the collecting electrode and shielding were examined at different rates of carriage movement as shown in FIGS. 12 and 13. Faster carriage movement (here, 375 mm/sec) provided a sharper drop-off rate and reasonably good uniformity relative to a slower carriage rate (here, 250 mm/sec). Rates can thus be adjusted to optimize deposition characteristics for a given polymer or polymer/biologically active agent combination. FIG. 17 shows the results of an experiment in which the rate of air flow was increased. Typically, the airflow rate used in the experiments described herein was ˜50 m3/hour, and was determined by a differential in inlet vs. outlet air flow, where inlet is 0 and outlet is 47-48. It is believed, without wishing to be bound or limited by theory, that when the inlet vs. outlet airflow differential is much higher, like 100 to 150 m3/hour, the cross flow of air (perpendicular to nanofiber trajectory in the machine direction) causes unequal drift of nanofiber collection on the substrate in the cross-direction. Some airflow is needed for water/solvent evaporation but too much can be detrimental.

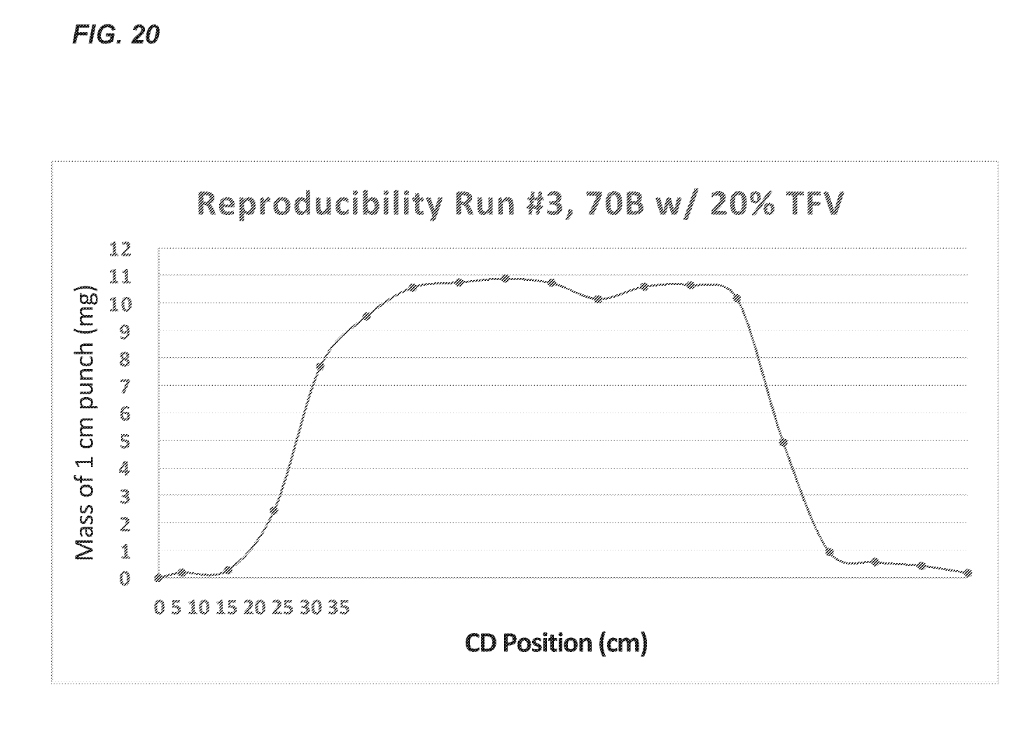

In FIG. 20, it was investigated whether the addition of a biologically active agent would alter the uniformity of non-woven nanofiber fabric deposition. Parameters used were as follows: 142 4×4 cm samples—X=251 grams per square meter (gsm), COV=2.2%−4 columns in machine direction (MD) using 25 cm carriage length with a yield: mass within 90% max. BW/total mass=73%. This effectively doubled previous typical yields—Using shielding, high BW and long MD (152.5 cm)—Loading efficiency: X=20.1 mg TFV/100 mg fiber—Encapsulation efficiency: X=101%, COV=1.5%; Yield target is >80%. This employed all the insulation and shielding technology described to make 100+4×4 cm samples loaded at clinical drug dosing to prove the scale up manufacturing of the nanofiber device at high BW, 250 gsm. TFV is the antiretroviral tenofovir and 70B is a rapid release polymer blend of PVA and PEO. This experimental run demonstrated a doubling in mass of on grade 4×4 samples (yield) relative to previous experimental runs and the uniformity of drug content in those samples is excellent with a low coefficient of variance of 1.5%, when 5% is acceptable. Results demonstrate 73% mass of samples within 90% max basis weight relative to the total mass of fiber electrospun.

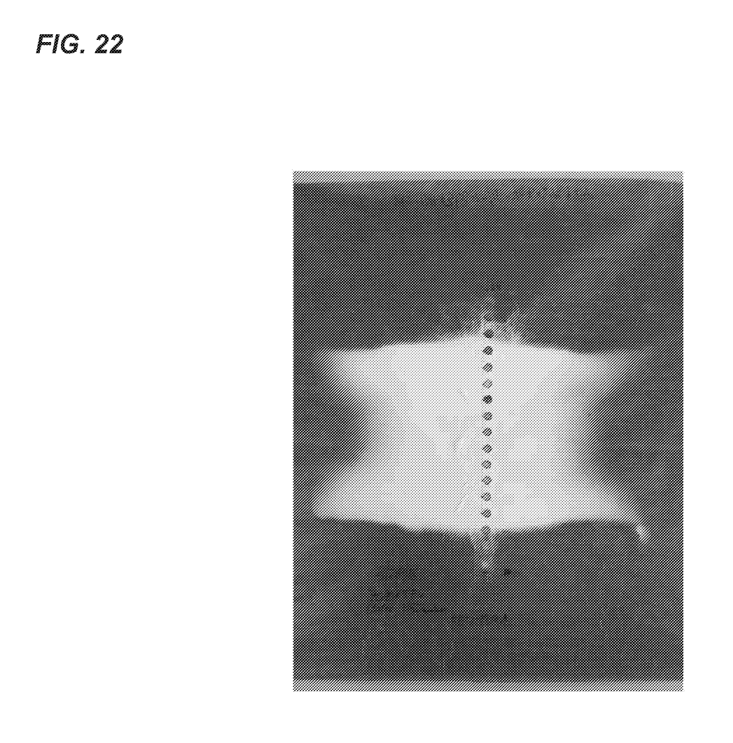

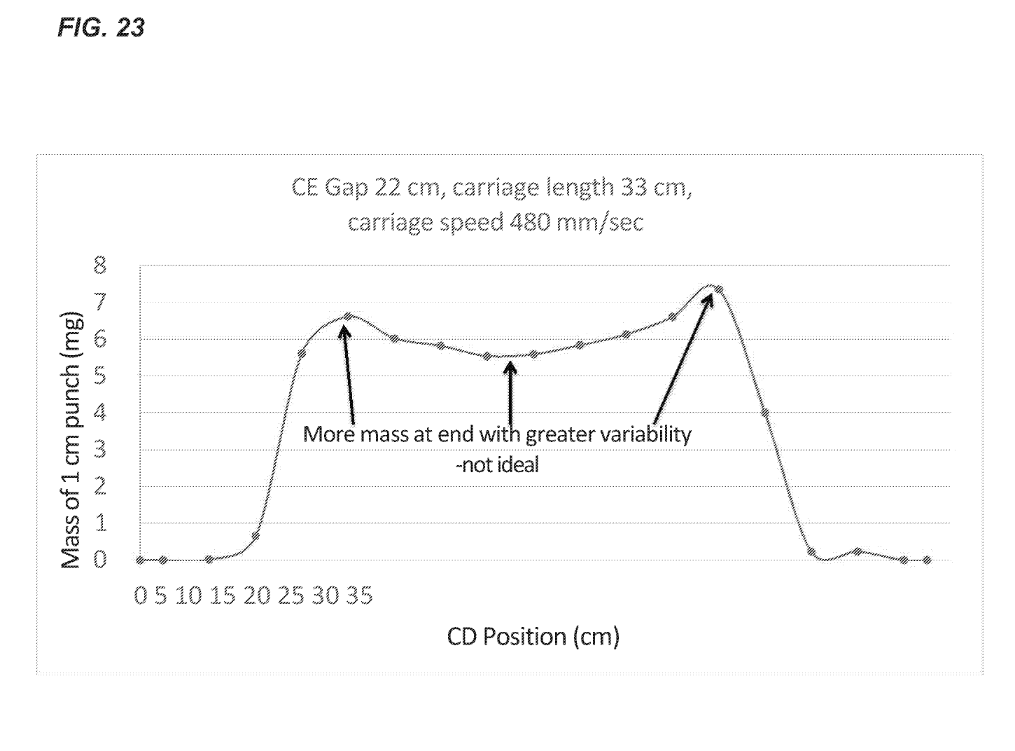

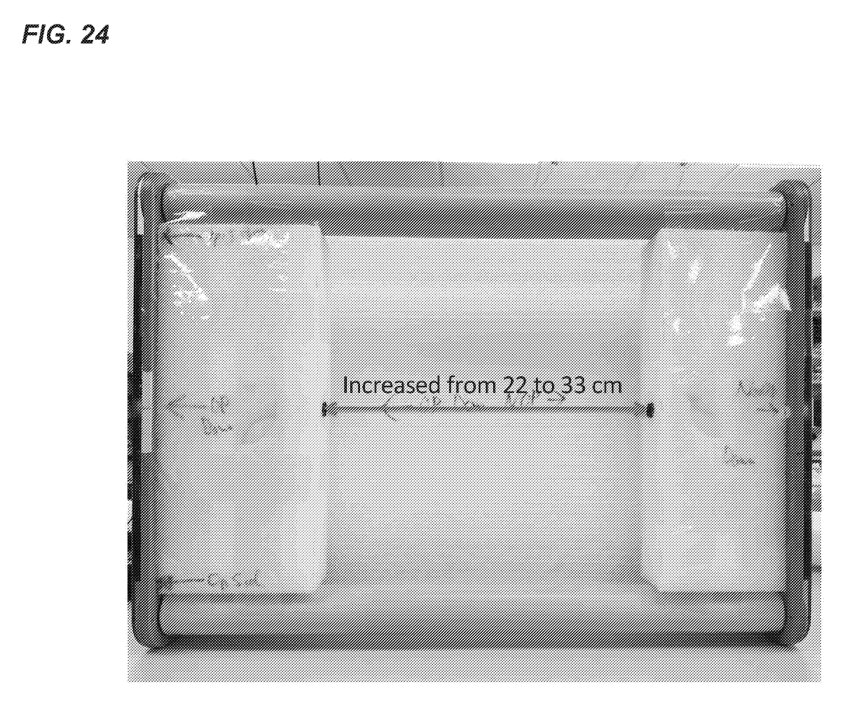

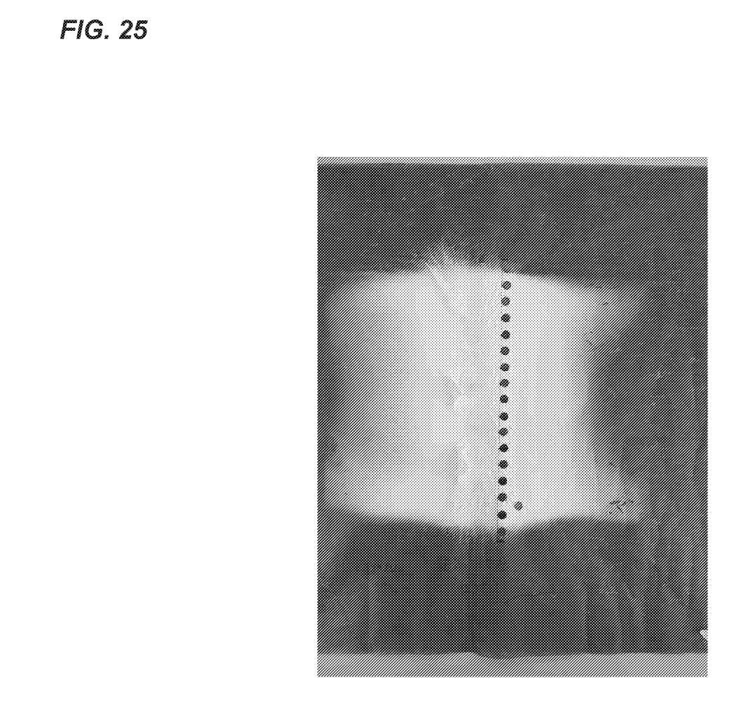

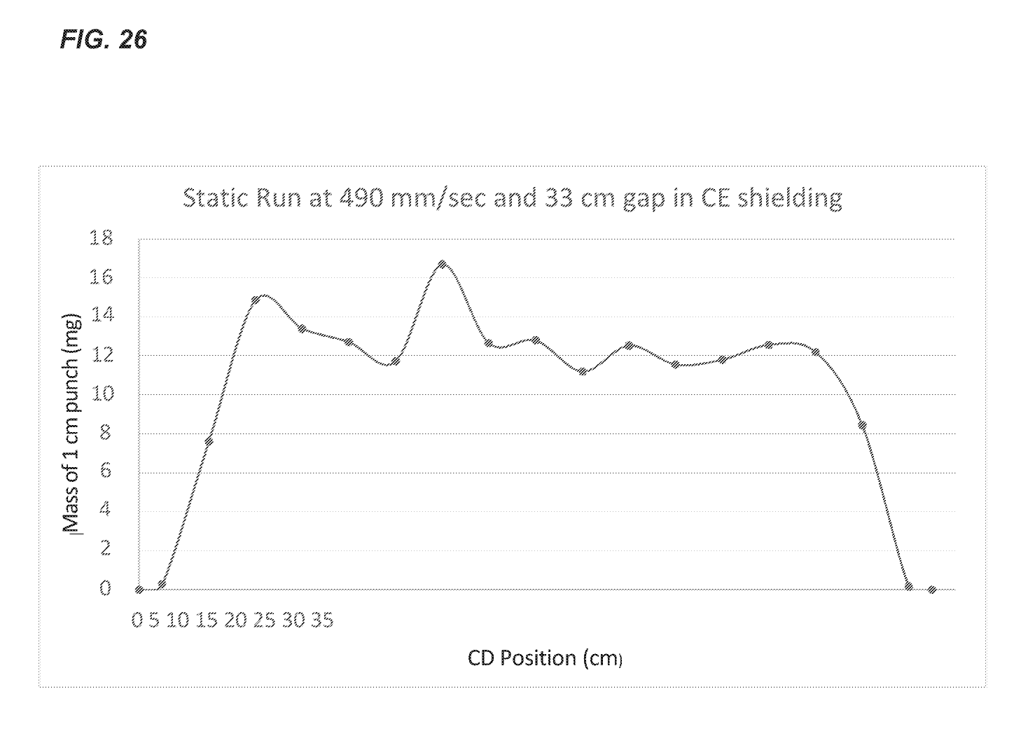

The potential effect on uniformity of nanofiber deposition caused by varying the carriage length of the electrospinning electrode relative to the exposed portion or bare gap on the collecting electrode was also examined. FIG. 21 shows a schematic of an experimental arrangement using polyethylene shielding 80a and 80b and butyl rubber insulation 60a and 60b (set out of sight within the polyethylene shielding in the view shown) providing a 22 cm bare gap on the collecting electrode 10 over a 33 cm carriage length of the electrospinning electrode 30. The resulting fiber deposition pattern is shown in FIG. 22. This approach provided more fiber mass at the ends but greater variability over the deposited nanofiber fabric—see FIG. 23. FIG. 24 shows an embodiment in which the bare gap on the collecting electrode 10 was increased to 33 cm and used with the 33 cm electrospinning electrode carriage length. The resulting nanofiber fabric deposition is shown in FIG. 25, and mass distribution graphically represented in FIG. 26.

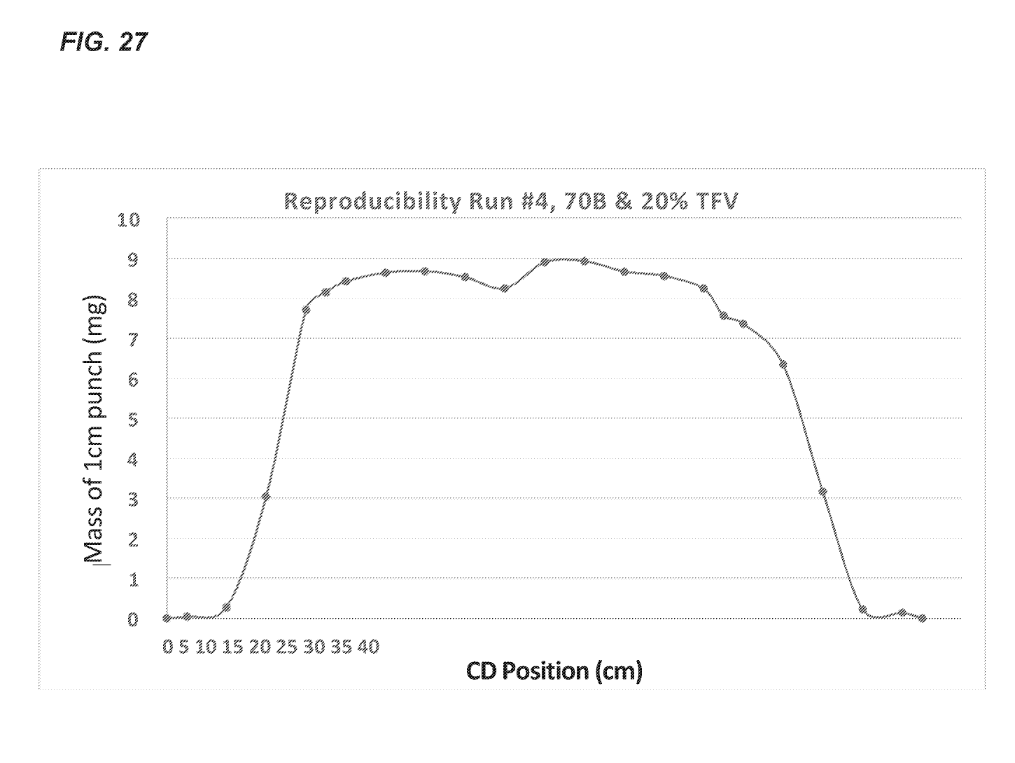

FIG. 27 shows the results when 162 4×4 cm samples were used where X=99 gsm, COV=2.3%. 5 columns were used in MD using 33 cm carriage length giving a yield of mass w/i 90% Max BW/total mass=57%. These results were a lower BW than Run #3 (FIG. 22) and less contiguous samples. Loading efficiency: X=19.8 mg TFV/100 mg fiber. Encapsulation efficiency: X=99%, COV=1.6%. This reproducibility experimental run (#4) demonstrates that 5 MD columns of 4×4 cm samples can be made where only 4 MD columns were made in reproducibility run #3. This was done without the collimating PE side shields, and only increasing the exposed CE and SE lengths from 25 to 33 cm. The resulting yield was 57%. When collimating shielding is employed with higher BW, the 80% target yield is expected to be achieved.

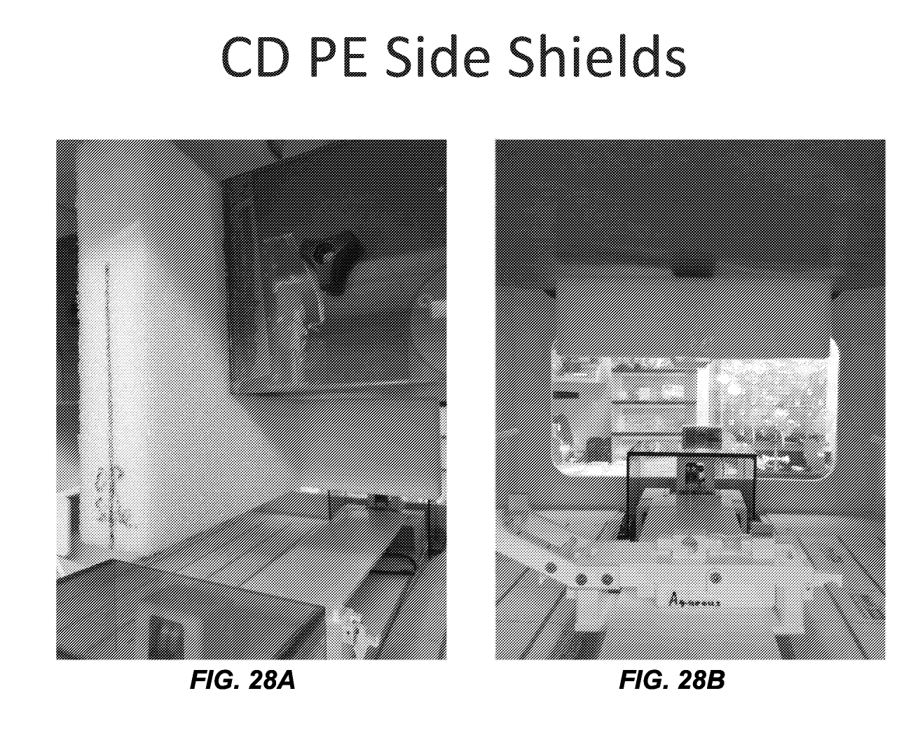

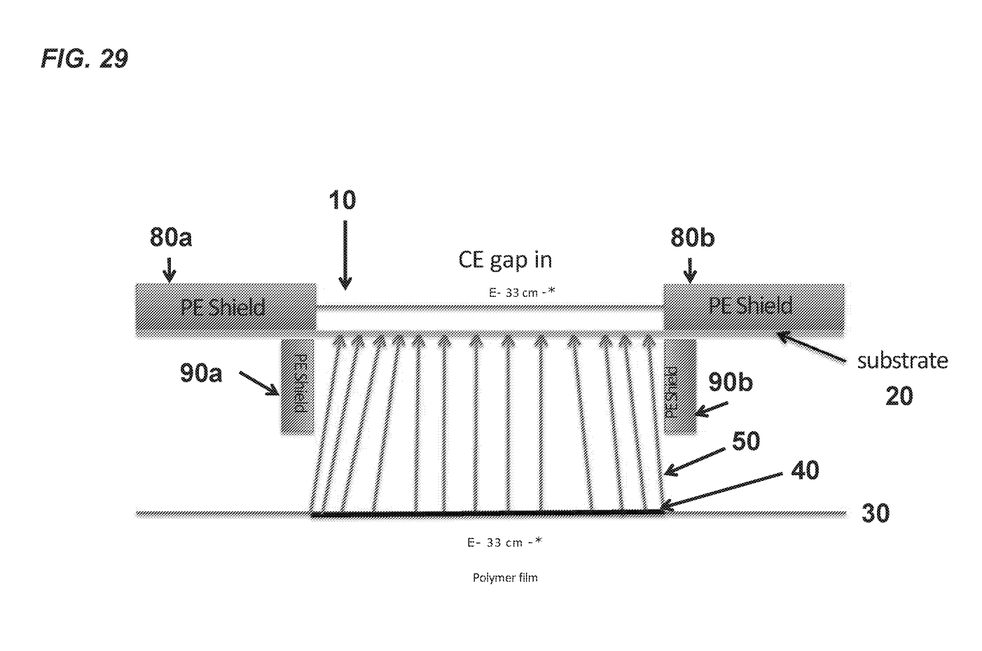

It was also considered that shielding between the electrospinning electrode and the substrate further influences the uniformity of non-woven nanofiber fabrics produced by electrospinning To this end, the effect of collimating side shields was examined. FIG. 28 shows photographs and FIG. 29 shows a schematic of an embodiment in which collimating side shields 90a and 90b were added adjacent to the substrate 20, between the substrate 20 and electrospinning electrode 30. The collimating side shields 90a and 90b are aligned with the edges of the shielding 80a and 80b facing the bare, 33 cm unshielded gap on the collecting electrode 10. Preferably, the collimating shields extend between 30% to 50%, inclusive, of the distance from the bottom of the substrate to the spinning electrode. In some embodiments, the collimating shields extend at least 30%, at least 35%, at least 40%, at least 45%, at least 50% or between 30% to 50%, inclusive, between 35% to 50%, between 40% to 50%, inclusive, between 45% to 50%, inclusive, of the distance from the bottom of the substrate to the spinning electrode.

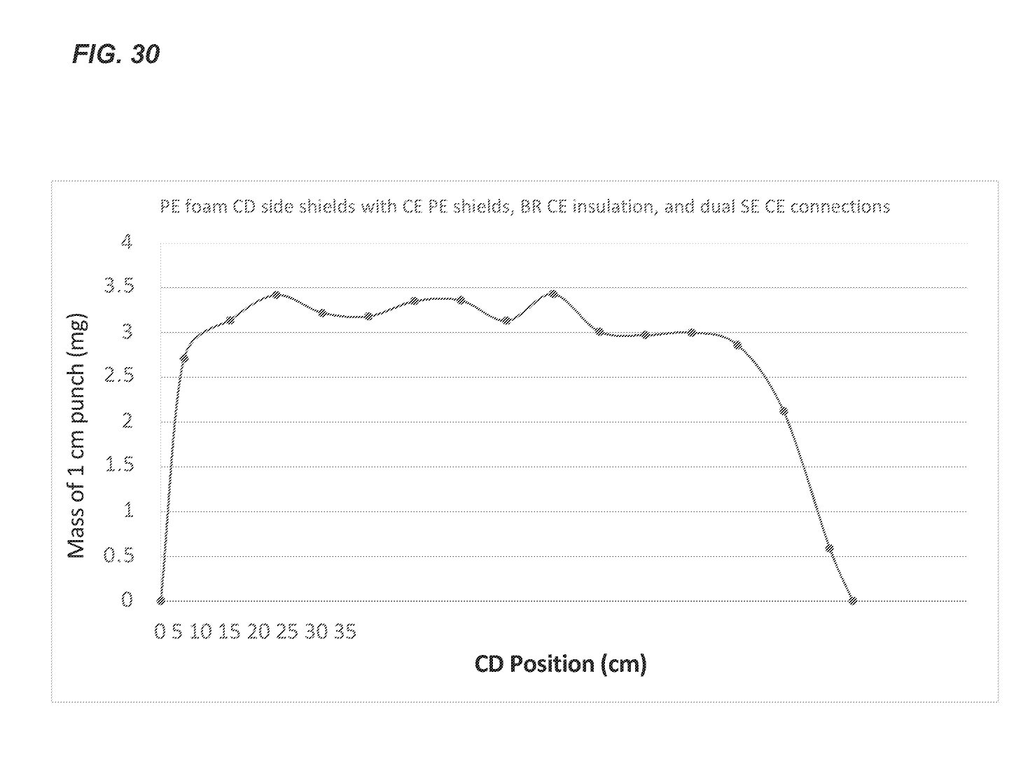

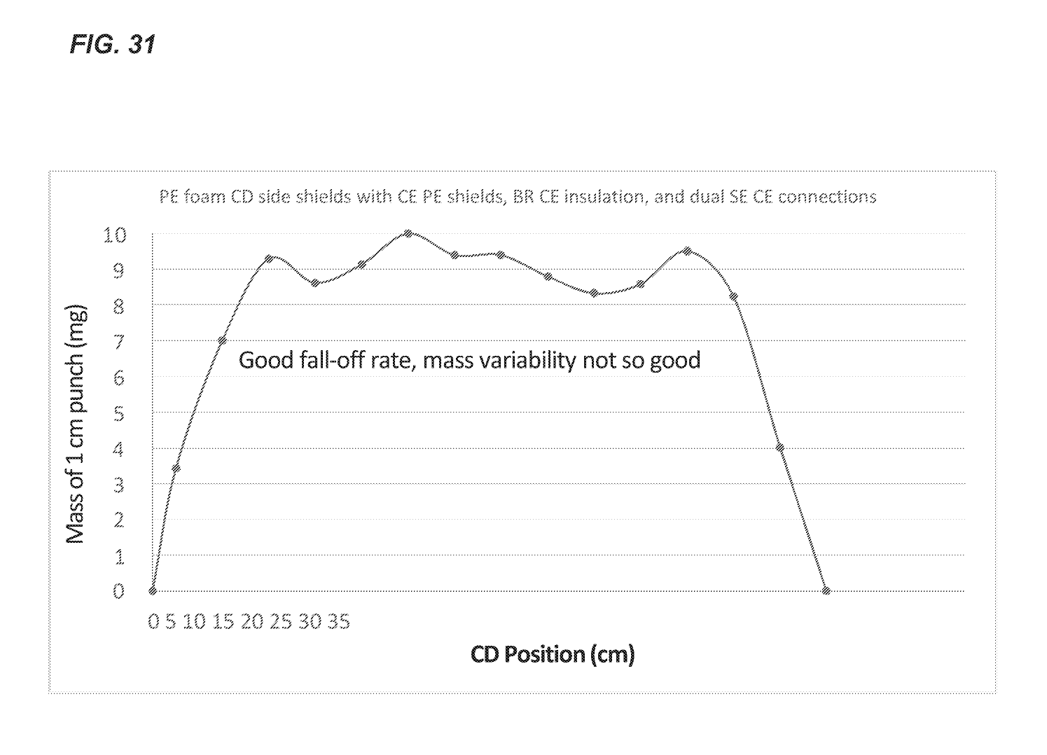

The effects of an embodiment using polyethylene (PE) collimating side shields with PE shielding of the collecting electrode, butyl rubber insulation on the collecting electrode and dual electrical connections to the electrospinning electrode and collecting electrode are shown, for example, in the mass distribution graphs in FIGS. 30 and 31. The collimating side shields improved the fall-off rate at the edges of the nanofiber fabric mat. FIG. 30 shows results with 8 machine direction (MD) passes, and FIG. 31 shows results with 20 machine direction passes.

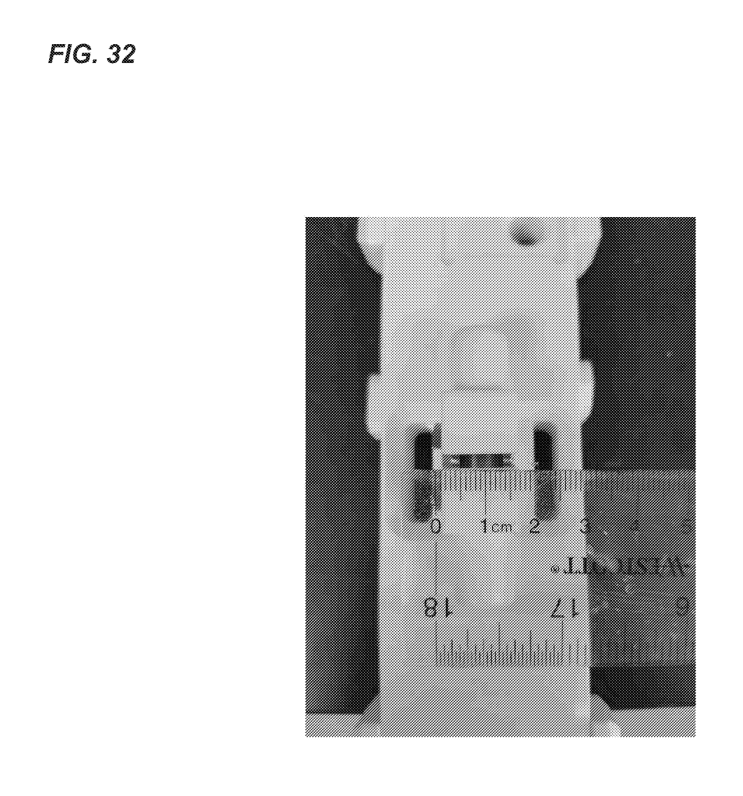

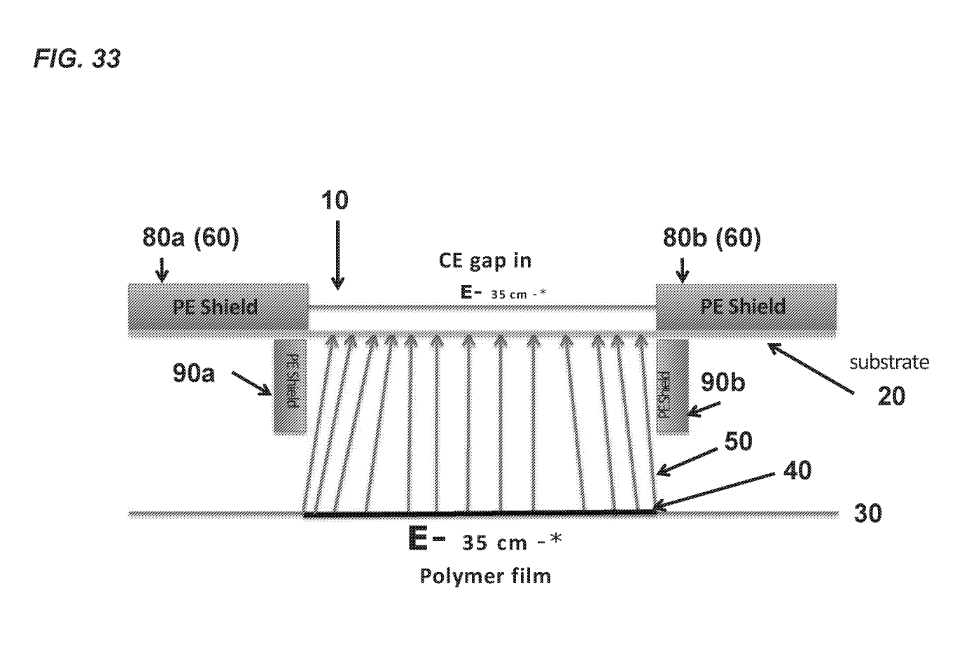

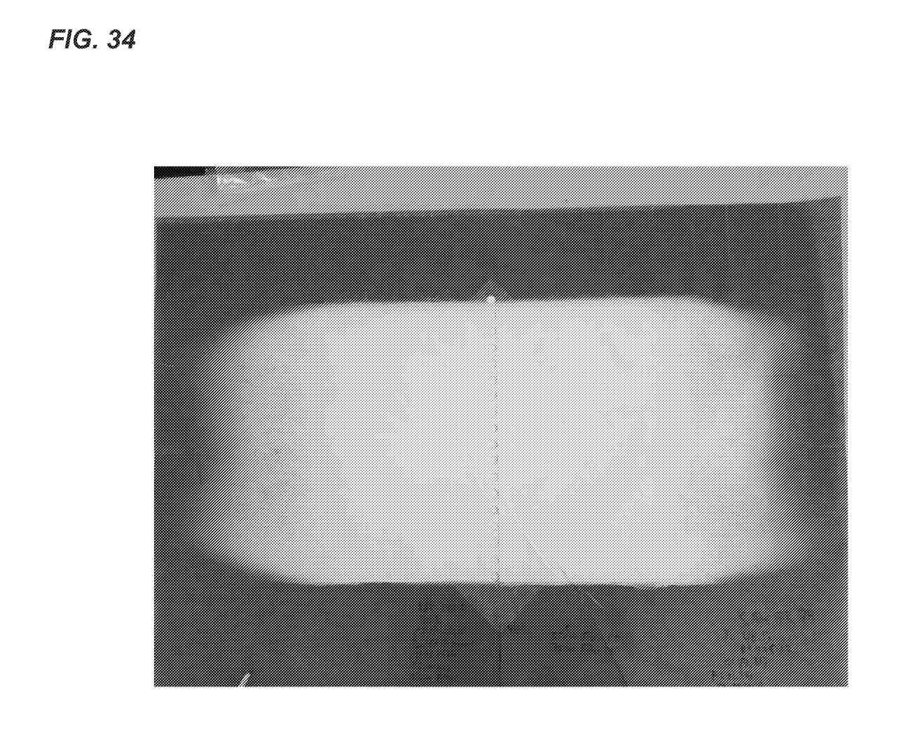

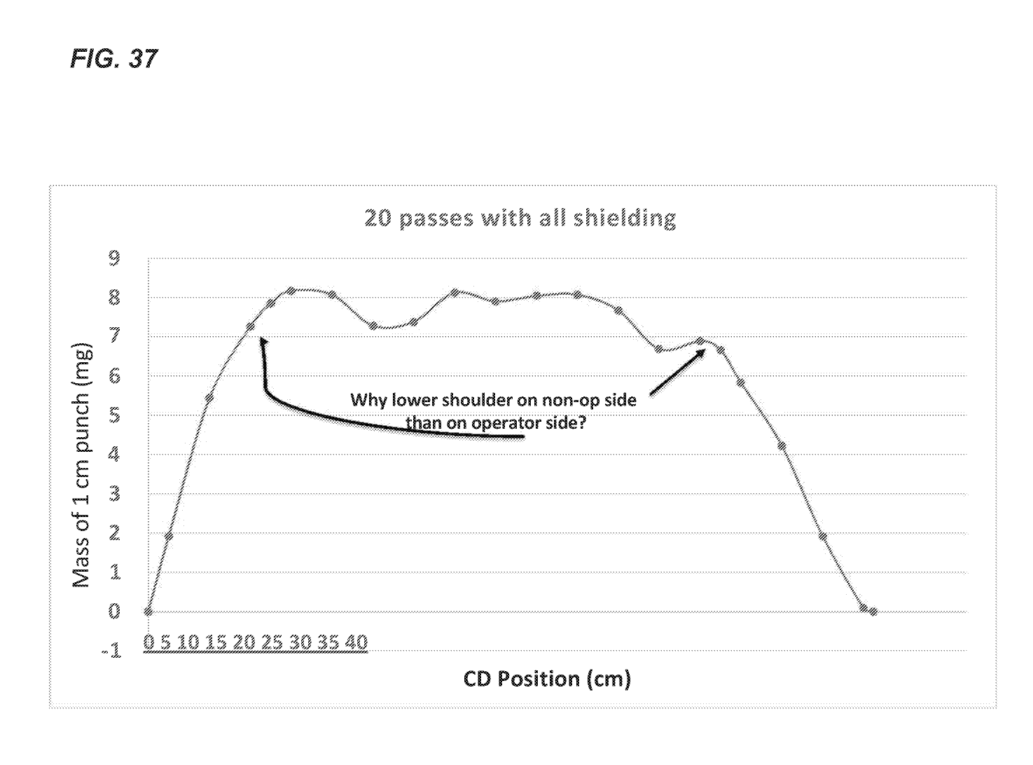

It was noted that fibers accumulated on the face of the collimating side shields. It was determined that the actual length of polymer deposited on electrospinning electrode is 35 cm when carriage length is set to 33 cm, because the carriage insert adds about 1 cm of polymer deposited on each end of the electrospinning electrode. See FIG. 32, which shows the dimension of the carriage insert. In FIG. 33, the collecting electrode gap is optimized to the actual electrospinning carriage length by accounting for the approximately 1 cm added on each end of the 33 cm electrospinning carriage length by the carriage insert. In FIG. 34, the fiber deposition footprint using polyethylene collimating side shields, along with BR insulation, PE CE shielding, dual CE and SE connection and PE collimating side shields is shown. See also FIG. 37.

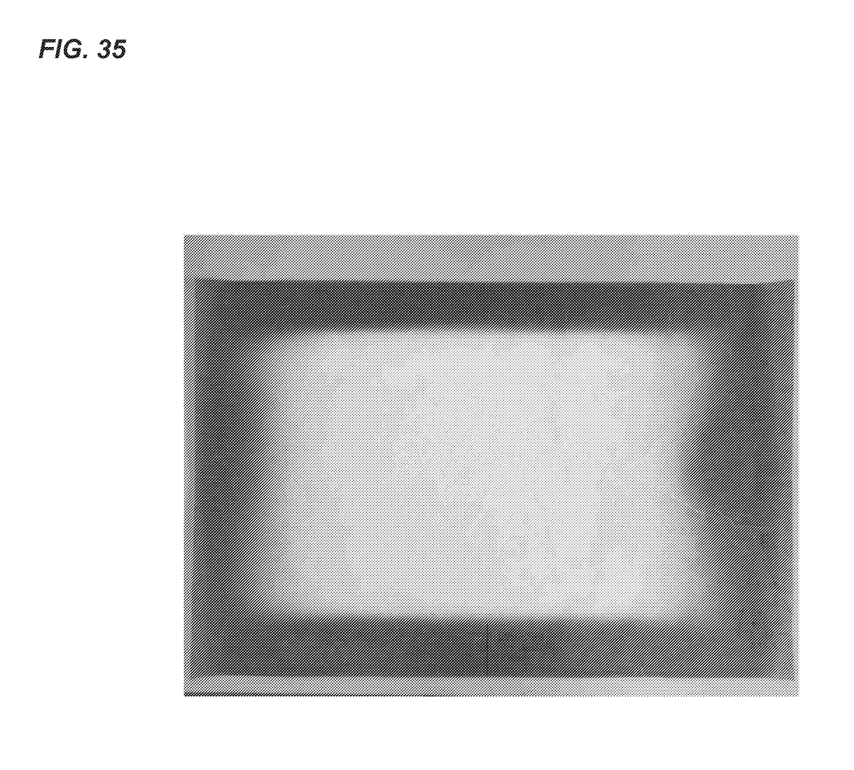

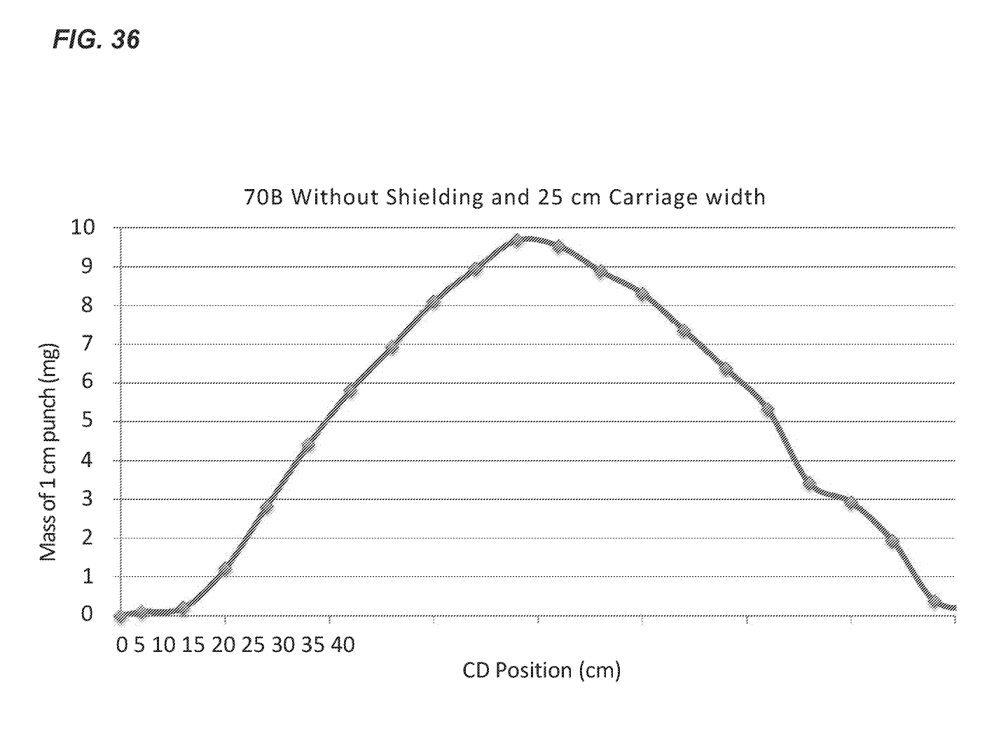

FIGS. 35 and 36 show a picture and CD mass profile of 70B polymer blend electrospun without any insulation, shielding or collimating side shields. The dual CE and SE connections are being used but their effect was not realized, indicating, without wishing to be bound or limited by theory, that their effect is additive to the principle insulation and shielding effects. FIG. 35 shows that a material can appear uniform visually, when indeed it is not as shown by the depiction of mass profile of the same material in FIG. 36.

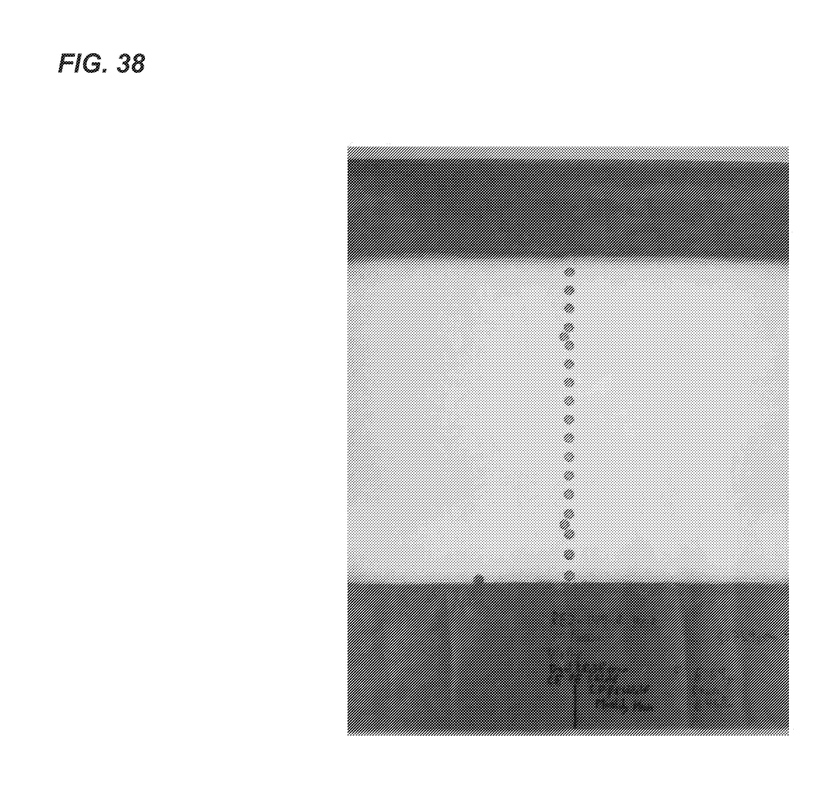

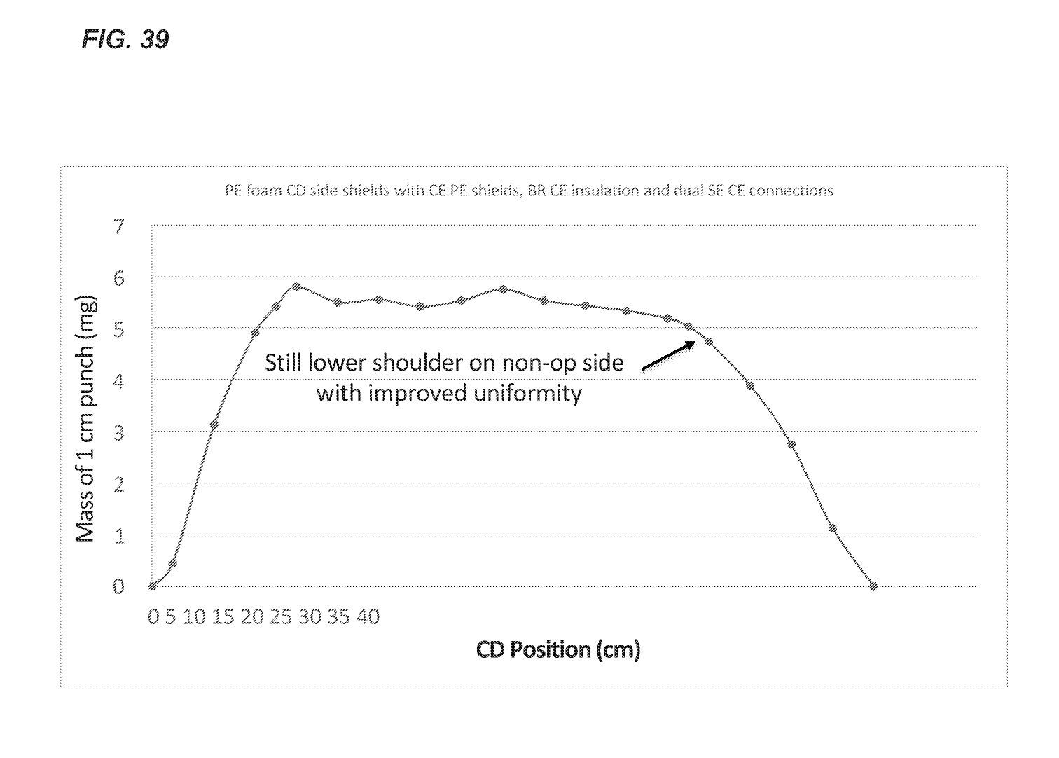

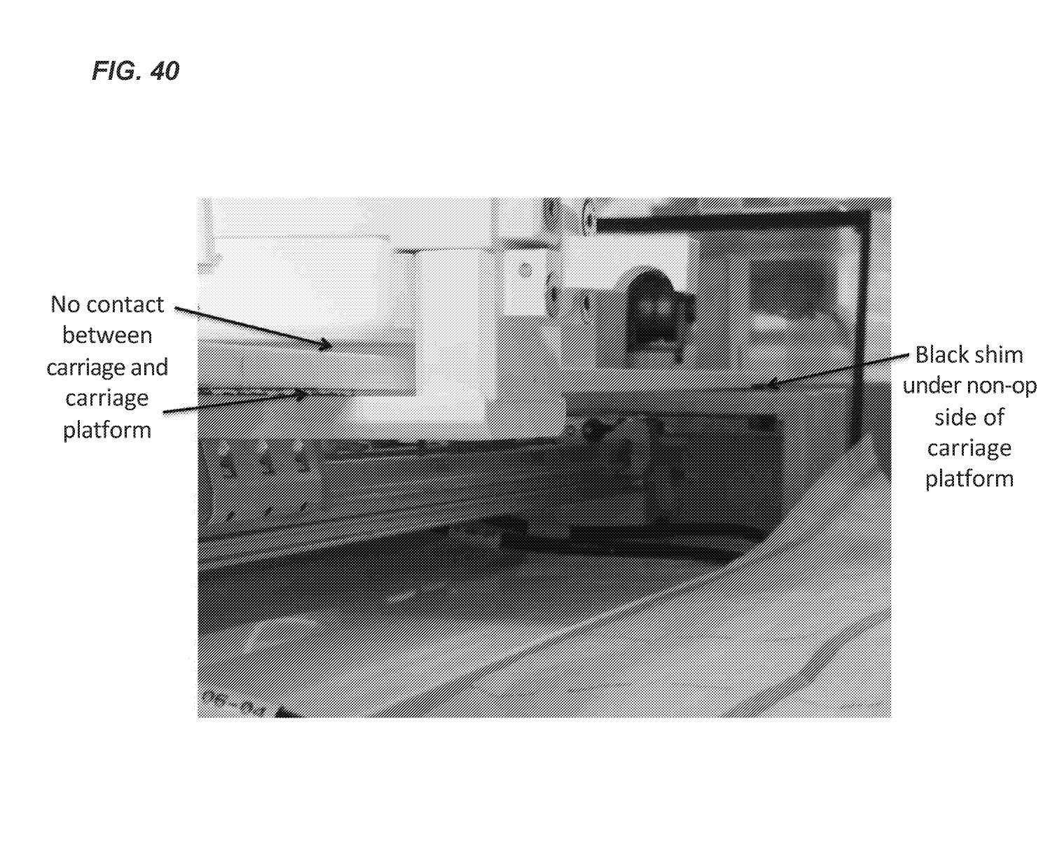

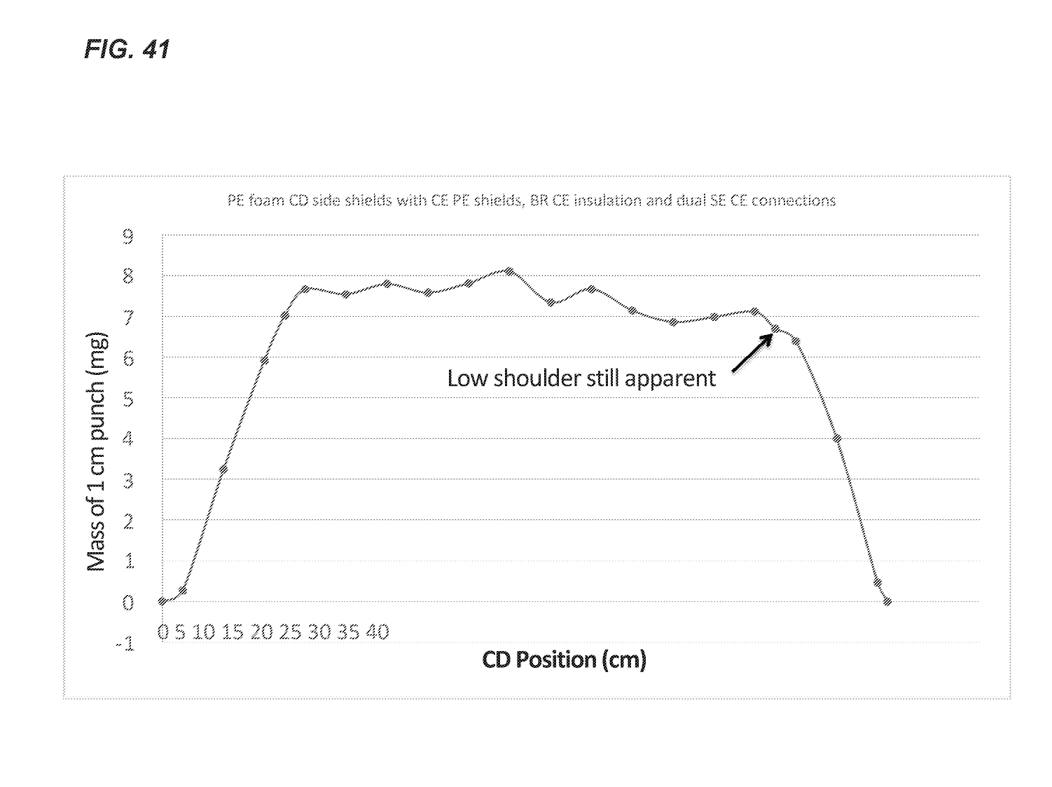

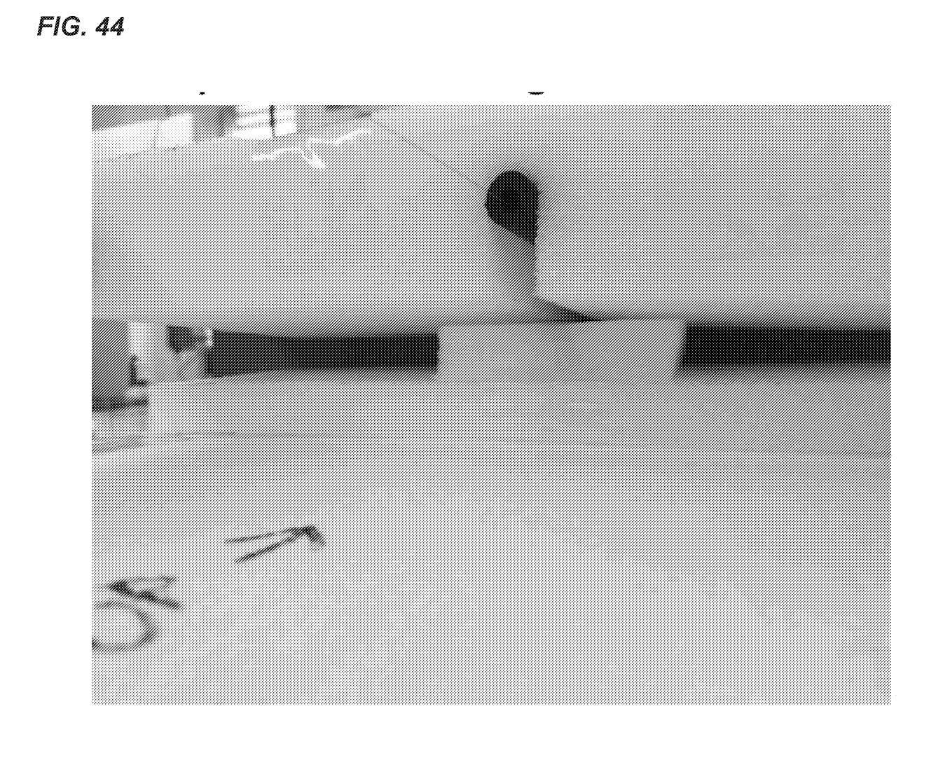

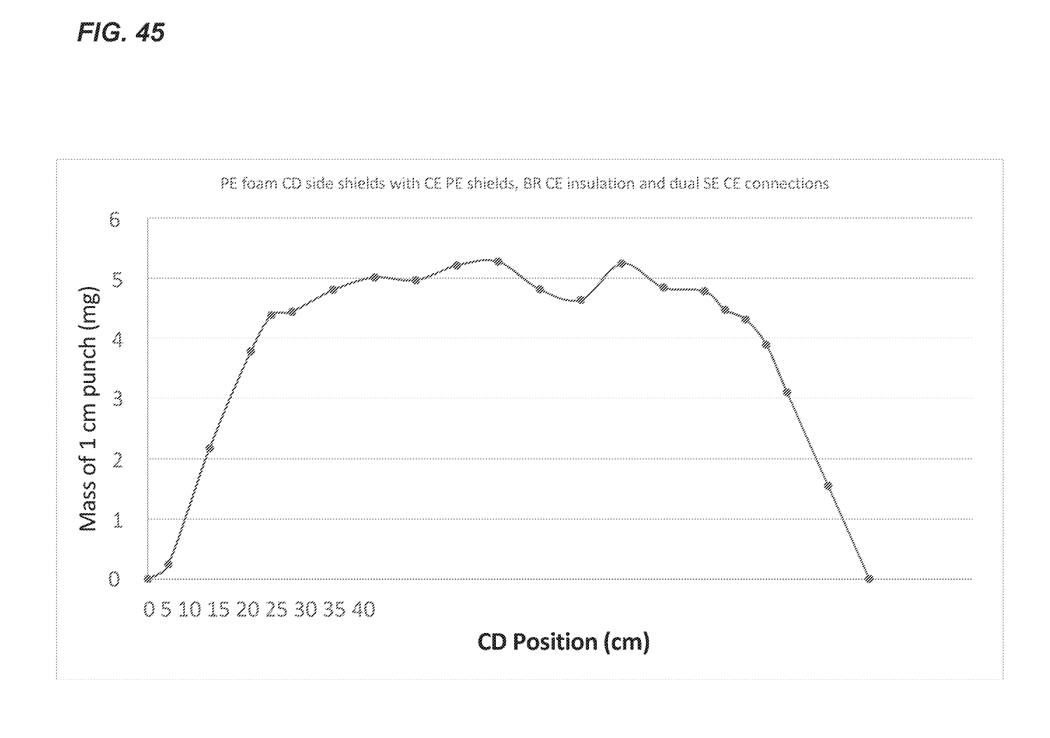

While the modifications including insulation and various configurations of shielding clearly provided significant improvements in uniformity, it was noted that certain other factors could also further influence uniformity. These factors are investigated and shown in FIGS. 38-41 and include, for example, the benefit of regularly (e.g., monthly) cleaning and lubricating the device and adjusting the carriage. This attention to detail improved/maintained uniformity. It was noted that the drop-off in mass distribution was less steep on one side relative to the other despite the use of shielding, insulating materials, and additional electrical connections on the electrodes as described herein. See, e.g., FIG. 39. The causes of this were investigated, and it was found, for example, that the carriage was contacting the carriage platform on one side; a shim was added to prevent carriage/platform contact, and the impact on carriage speed was removed. The improvement provided is evident in FIG. 41. Similar investigation of the need (or lack of need) for such fine tuning is shown in FIGS. 44 and 45. Such factors are discussed to provide guidance for the ordinarily skilled artisan regarding certain sources of variability and ways to correct for or overcome them, such that uniformity of non-woven nanofiber fabrics as described herein can be achieved and maintained over serial production runs.

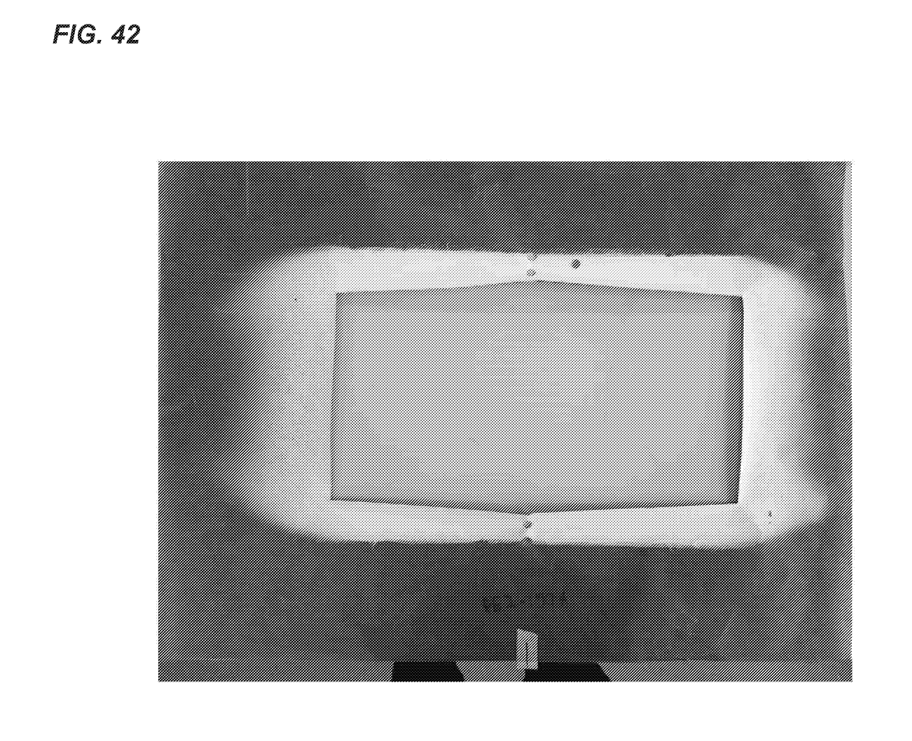

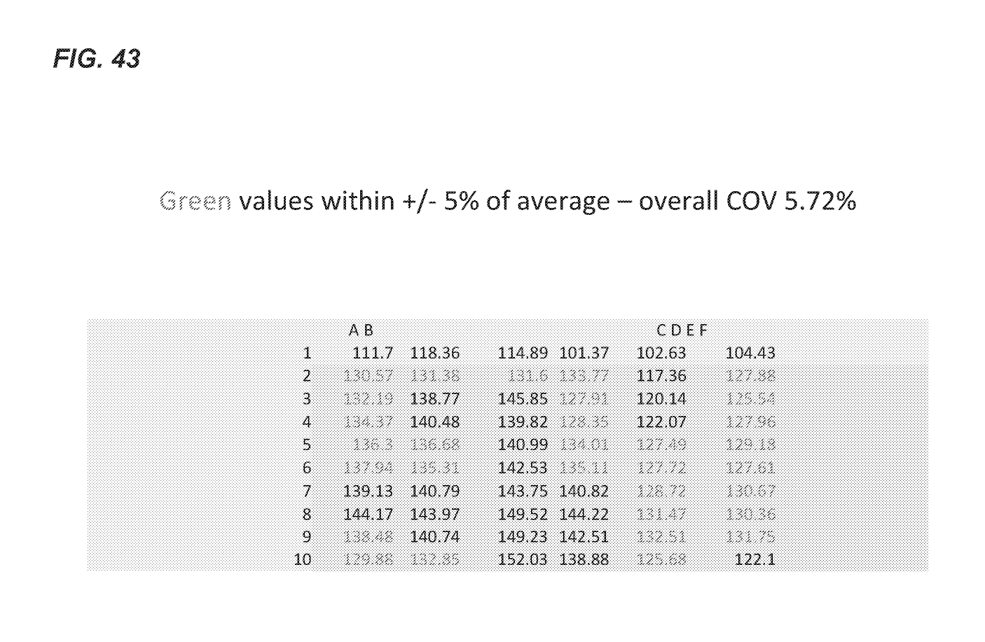

FIGS. 42 and 43 show examples of results and uniformity achieved using the modifications and parameters described herein. The area of uniform nanofiber deposition is clearly increased relative to that achievable without any or all of the insulation, shielding and electrical connection modifications described herein.

### Nanofiber Fabric Compositions and Methods Thereof

Provided herein are novel polymer nanofiber fabric compositions and biologically active agent delivery compositions based on the discoveries described herein that allow, in part, for the production and fabrication of nanofiber fabric compositions having significantly increased uniformity over a larger area and high basis weights.

“Nanofibers” refers to fibers having high aspect ratios (aspect ratio>10:1) and diameters or cross-sections generally less than about 1 um, typically varying from about 20 nm to about 1000 nm, i.e., less than 1000 nm, less than 900 nm, less than 800 nm, less than 700 nm, less than 600 nm, less than 500 nm, less than 400 nm, less than 300 nm, less than 200 nm, less than 100 nm, less than 75 nm, or less than 50 nm. Nanofibers useful in embodiments of the aspects described herein are typically fabricated by electrospinning, which applies electrostatic forces for formation of nanoscale polymer fibers fabricated into fabrics of varying geometries. Electrospinning exploits the interplay between a polymer solution's viscosity, surface tension, and conductivity in an electric field. Polymer nanofibers synthesized by electrospinning have consistent diameters and morphology, which can be controlled by modulating the solution and process parameters, such as concentration and electric field strength. In some embodiments of the compositions described herein, the electrospinning is performed using a nozzle-less electrospinning method as described elsewhere herein.

However, as shown herein, the ability to produce non-woven nanofiber fabrics having both a high basis weight and consistent uniformity over larger areas has not been achieved, thus making it difficult to apply nanofiber fabrics to large-scale industrial manufacturing and applications requiring uniformity and consistency, such as biomedical applications. For example, using a NANOSPIDER™ apparatus, the width of nanofiber fabric produced is, at a maximum, 1.6 meters or 160 cm, with a mean basis weight between 0.03-50 gsm, and having a standard deviation of±30%. Accordingly, by providing both high basis weight and consistent uniformity, the nanofiber fabric compositions described herein are significantly different from those in the art and have increased industrial utility and applicability, for example, in biomedical applications, requiring consistency and accuracy.

The nanofiber compositions described herein have a high basis weight. Basis weight is a term of art used to refer to the mass per square meter of a given fabric. As used herein, “high basis weight” refers to nanofiber fabrics having a real mass between 50 and 500 grams per square meter (gsm or g/m²), as measured on a dry basis, (i.e., after the residual solvent has evaporated or been removed), typically at least 50 gsm, at least 60 gsm, at least 70 gsm, at least 80 gsm, at least 90 gsm, at least 100 gsm, at least, 110 gsm, at least 120 gsm, at least 130 gsm, at least 140 gsm, at least 150 gsm, at least 160 gsm, at least 170 gsm, at least 180 gsm, at least 190 gsm, at least 200 gsm, at least 250 gsm, at least 300 gsm, at least 350 gsm, at least 400 gsm, at least 450 gsm, at least 500 gsm, or more. Thus, in some embodiments of the compositions described herein, the basis weight of the nanofiber fabric is in the range of 50-500 gsm, inclusive.

In addition, the nanofiber fabrics described herein are “uniform,” by which it is meant that the nanofiber fabrics have a high degree of fabric homogeneity such that the basis weight at all locations is within ±10% of the mean basis weight. Basis weight uniformity can be expressed in terms of the percent coefficient of variation (CV % or COV %) for the distribution of basis weight, and is typically computed after measuring the mass of numerous samples of identical area.

Accordingly, in some aspects, provided herein are uniform high basis weight, non-woven, polymer nanofiber fabric compositions.

As demonstrated herein, one significant advantage of the devices, compositions, and methods provided herein is the ability to make nanofiber fabrics having increased uniformity over a larger area compared to those devices and methods known in the art. As used herein, “uniform area” means that within a given area of the deposited nanofiber fabric or mat there is a high degree of fabric homogeneity, such that the basis weight at all locations within the given area is within ±10% of the mean basis weight of that area. Accordingly, in some embodiments of the nozzle-less electrospinning devices described herein, the uniform area of the electrospun polymer mat or fabric deposited on the substrate by the device in the presence of one or more, and up to all of the insulating, shielding, electrical contact addition and process improvements described herein is at least 25 cm in the CD dimension, or more, including, for example, at least 30 cm wide, at least 35 cm wide, at least 40 cm wide, at least 45 cm wide, at least 50 cm wide, at least 55 cm wide, at least 60 cm wide, at least 65 cm wide, at least 70 cm wide, at least 75 cm wide, at least 80 cm wide, at least 85 cm wide, at least 90 cm wide, at least 95 cm wide, at least 100 cm wide, at least 105 cm wide, at least 110 cm wide, at least 115 cm wide, at least 120 cm wide, at least 125 cm wide, at least 130 cm wide, at least 135 cm wide or even 140 cm wide in the CD dimension. The length of such fabrics in the MD dimension depend upon the length of substrate drawn between the electrodes during the production run and can be, for example, at least 100 cm, at least 200 cm, at least 300 cm, at least 400 cm, at least 500 cm or more in length in the MD dimension, including, for example, at least 600 cm, at least 700 cm, at least 800 cm, at least 900 cm or even 1000 cm in the MD dimension. The sizes of uniform, high basis weight nanofiber polymer fabrics that can be produced using improvements described herein can therefore be in the range of at least 25 cm by 100 cm to as much as 140 cm by 1000 cm (or more in the MD dimension). These sizes can be achieved using, for example, a 1.6 m NANOSPIDER™ device or its equivalent modified and used with processes as described herein. Included, for example, are uniform, high basis weight nanofiber polymer fabrics of at least 30 cm (CD dimension) by 100 cm (MD dimension), at least 35 cm by 100 cm, at least 40 cm by 100 cm, at least 45 cm by 100 cm, at least 50 cm by 100 cm, at least 55 cm by 100 cm, at least 60 cm by 100 cm, at least 65 cm by 100 cm, at least 70 cm by 100 cm, at least 75 cm by 100 cm, at least 80 cm by 100 cm, at least 85 cm by 100 cm, at least 90 cm by 100 cm, at least 95 cm by 100 cm, at least 100 cm by 100 cm, at least 105 cm by 100 cm, at least 110 cm by 100 cm, at least 115 cm by 100 cm, at least 120 cm by 100 cm, at least 125 cm by 100 cm, at least 130 cm by 100 cm, at least 135 cm by 100 cm and at least 140 cm by 100 cm. Similar, essentially proportional, improvements in uniform areas of high basis weight nanofiber polymer fabrics are also achievable using smaller free surface electrospinning devices, such as the 0.5 m and 1.0 m NANOSPIDER™ devices known in the art, or their equivalent, by applying the teachings provided herein.

The uniformity of the compositions described herein can be measured or determined by obtaining samples of defined size over various points of a given area of the nanofiber fabric and determining the weights of each such sample. See, for example, U.S. Pat. No. 5,173,356, which is hereby incorporated by reference in its entirety and describes collecting small swatches taken from various locations across the width of the web (sufficiently far enough away from the edges to avoid edge effects) to determine a basis weight uniformity. Additional acceptable methods for evaluating uniformity can be practiced in accordance with “Nonwoven Uniformity-Measurements Using Image Analysis,” disclosed in the Spring 2003 International Nonwovens Journal Vol. 12, No. 1, also incorporated by reference in its entirety. Thus, for example, one of skill in the art can measure the weight of 1 cm discs or 1 cm² areas obtained from a various spots or positions over a total area of nanofiber fabric of between 5 cm²-200 cm² and measure whether the weight of each of those 1 cm discs or 1 cm² areas is within ±10% of the mean basis weight over the entire area of at least 5 cm²-200 cm².

Non-limiting examples of suitable polymers for use in the compositions described herein include poly(lactide-co-glycolide) (PLGA), polylactic acid (PLA), poly c-caprolactone (PCL), polyvinyl alcohol (PVA), polyethylene oxide (PEO), polyvinylpyrrolidone (PVP), poly methacrylic acid (PMAA) and ethyl cellulose (EC).

Suitable polymers for use in the compositions described herein can further be qualified as water soluble polymers; polymers that require on-contact cross-linking, and/or polymers that cannot be readily dissolved at a high enough concentration to provide sufficient viscosity for random entanglement and solvent evaporation to form polymeric fibers, and/or polymers that require precipitation, and/or polymers dissolved in water at low concentrations (e.g., below 2%) and/or polymers that require both extension in air and precipitation (e.g., polyamides, e.g., liquid crystalline polymers). As used herein, the term “water soluble polymer” is intended to denote a polymer that is soluble in water such that at least 50% by weight of the polymer dissolves in water when immersed in 10 or more times its own weight of water for ample time (e.g., 24 hours or longer) at ambient temperature and atmospheric pressure. Synthetic water-soluble polymers refer to synthetic substances that dissolve, disperse or swell in water and, thus, modify the physical properties of aqueous systems in the form of gelation, thickening or emulsification/stabilization. These polymers usually have repeating units or blocks of units- the polymer chains contain hydrophilic groups that are substituents or are incorporated into the backbone. The hydrophilic groups may be nonionic, anionic, cationic or amphoteric. As used herein, the term “water insoluble polymer” is intended to denote a polymer that is sparingly soluble in water such that at least 80% by weight of the polymer does not dissolve in water when immersed in 10 or more times its own weight of water for ample time (e.g., 24 hours or longer) at ambient temperature and atmospheric pressure.

Examples of water soluble polymers include naturally occurring polymers, such as mucopolysaccharides, such as pullulan, hyaluronic acid, chondroitin sulfate, poly-y-glutamic acid, modified corn starch, β-glucan, gluco-oligosaccharides, heparin, and keratosulfate; cellulose, pectin, xylan, lignin, glucomannan, galacturonic acid, psyllium seed gum, tamarind seed gum, gum arabic, tragacanth gum, modified corn starch, soybean water-soluble polysaccharides, alginic acid, carrageenan, laminaran, agar (agarose), fucoidan, methyl cellulose, hydroxypropyl cellulose, and hydroxypropylmethyl cellulose; and water-soluble synthetic polymers, such as partially saponified polyvinyl alcohol (usable in the absence of a crosslinking agent), low-saponified polyvinyl alcohol, polyvinylpyrrolidone (PVP), polyethylene oxide, and sodium polyacrylate. These water soluble polymers can be used either individually or in combination of two or more thereof.

Polymers useful in generating the uniform high basis weight, non-woven, polymer nanofiber fabric compositions described herein can, in some embodiments, be further characterized as rapidly water soluble. As used herein, “rapidly water soluble,” when used in regard to a polymer, refers to a polymer having an aqueous solubility of at least such that at least 75% by weight of the polymer dissolves in water when immersed in 10 or more times its own weight of water for ample time (e.g., 24 hours or longer) at ambient temperature and atmospheric pressure. Non-limiting examples of rapidly water soluble polymers useful in the compositions described herein include polyvinyl alcohol (PVA), polyethylene oxide, polyvinylpyrrolidone (PVP), poly-2-ethyl-2-oxazoline, polyacrylic acid (PAA), polyethylene glycol (PEG), Polyacrylamides N-(2-Hydroxypropyl) methacrylamide (HPMA), and Divinyl Ether-Maleic Anhydride (DIVEMA).

Rapidly water soluble polymers useful in the uniform high basis weight, non-woven, polymer nanofiber fabric compositions described herein can also, in some embodiments, provide burst release of a biologically active agent. As used herein, the terms “burst release” or “burst kinetics” refer to the release of at least 50% of a given biologically active agent within 30 minutes or less of contacting or placement of a polymer nanofiber fabric compositions as described herein at or within a desired tissue or organ or other body site of a given organism or subject. In some embodiments of the compositions described herein, burst release can include release of at least 75% of a given a biologically active agent within 30 minutes, or at least 80%, at least 85%, at least 90%, at least 95% or even all of the biologically active agent (100%) within 30 minutes. In other embodiments, these levels of release are achieved, for example after 20 minutes or less, 15 minutes or less, 10 minutes or less, or even 5 minutes or less.

Polymers useful in generating the uniform high basis weight, non-woven, polymer nanofiber fabric compositions described herein can, in some embodiments, provide sustained release of a given biologically active agent. As used herein, the terms “prolonged release kinetics” or “sustained release kinetics” or “prolonged release” or “sustained release” refers to release of a given biologically active agent from a uniform high basis weight, non-woven, polymer nanofiber fabric composition over a period greater than 48 hours. In other words, it takes greater than 48 hours to achieve 100% release of the biologically active agent from the nanofiber composition. In some embodiments, sustained release can include, for example, release over 72 hours, over 84 hours, over 96 hours or more, including one week or more. Non-limiting examples of polymers providing sustained release useful in the compositions described herein include poly[lactic-co-glycolic] acid, polycaprolactone, ethyl cellulose, hydroxypropylmethyl cellulose (HPMC), hydroxypropyl cellulose (HPC), hydroxyethyl cellulose (HEC), and sodium carboxy methyl cellulose (Na-CMC).

The high basis weight and uniformity of the nanofiber compositions described herein are critical in allowing for their use in the delivery of biologically active agents—without this uniformity, biologically active agents cannot be reliably delivered or administered using nanofiber compositions, since the high degree of variability or non-uniformity typically seen with nanofiber compositions makes it impractical or inadvisable to use them in biomedical applications, such as the delivery compositions described herein.

Accordingly, provided herein, in some aspects, are biologically active agent-delivery compositions comprising any of the uniform high basis weight, non-woven, polymer nanofiber fabric compositions described herein. These biologically active agent-delivery nanofiber compositions allow for the delivery of one or more biologically active agents to a given location, such as a target tissue or organ, in a subject. As shown herein, the uniform and high basis weight characteristics of the nanofiber compositions described herein allow for uniform distribution of one or more, two or more, three or more, four or more, five or more, six or more, seven or more, eight or more, nine or more, or ten or more biologically active agents in the nanofiber fabric composition.

As used herein, the term “biologically active agent” refers to molecules, encompassing small molecule drugs, derivatives, analogs, and salts thereof, further including peptides, proteins, nucleic acids, carbohydrates, and other biological molecules, that have a biological activity when present or administered to a subject. Biologically active agents can include, but are not limited to, compounds that may be classified as medicines, organic and inorganic drugs, nutrients, vitamins, herbal preparations, and other agents that might benefit a human or animal. In general, such classifications include, but are not limited to, ACE inhibitors, adrenergics and anti-adrenergics, alcohol deterrents (for example, disulfiram), anti-allergies, anti-anginals, anti-arthritics, anti-infectives (including but not limited to antibacterials, antibiotics, antifungals, antihelmintics, antimalarials and antiviral agents), analgesics and analgesic combinations, local and systemic anesthetics, appetite suppressants, antioxidants, anxiolytics, anorexics, antiarthritics, anti-asthmatic agents, anticoagulants, anticonvulsants, antidiabetic agents, antidiarrheals, anti-emetics, anti-epileptics, antihistamines, anti-inflammatory agents, antihypertensives, antimigraines, antinauseants, antineoplastics, antioxidants, antiparkinsonism drugs, antipruritics, antipyretics, antirheumatics, antispasmodics, antitussives, adrenergic receptor agonists and antagonists, breath freshening agents (including but not limited to peppermint oil, spearmint oil, wintergreen oil and menthol), cardiovascular preparations (including anti-arrhythmic agents, cardiotonics, cardiac depressants, calcium channel blockers and beta blockers), cholinergics and anticholinergics, contraceptives, cough and cold preparations, diuretics, decongestants, growth stimulants, hormones including but not limited to androgens, estrogens and progestins, steroids and corticosteroids, hypnotics, immunizing agents, such as vaccines, immunomodulators, immunosuppresives, muscle relaxants, neurologically-active agents including anti-anxiety preparations, antidepressants, antipsychotics, psychostimulants, sedatives and tranquilizers, sore throat medicaments, sympathomimetics, vasodilators, vasoconstrictors, xanthine derivatives and combinations thereof.

Additional representative biologically active agents include, by way of example and not for purposes of limitation, bepridil, diltiazen, felodipine, isradipine, nicardipine, nifedipine, nimodipine, nitredipine, verapamil, dobutamine, isoproterenol, carterolol, labetalol, levobunolol nadolol, penbutolol, pindolol, propranolol, solatol, timolol, acebutolol, atenolol, betaxolol, esmolol, metoprolol, albuterol, bitolterol, isoetharine, metaproterenol, pirbuterol, ritodrine, terbutaline, alclometasone, aldosterone, amcinonide, beclomethasone dipropionate, betamethasone, clobetasol, clocortolone, cortisol, cortisone, corticosterone, desonide, desoximetasone, 11-desoxycorticosterone, 11-desoxycortisol, dexamethasone, diflorasone, fludrocortisone, flunisolide, fluocinolone, fluocinonide, fluorometholone, flurandrenolide, halcinonide, hydrocortisone, medrysone, 6a-methylprednisolone, mometasone, paramethasone, prednisolone, prednisone, tetrahydrocortisol, triamcinolone, benoxinate, benzocaine, bupivacaine, chloroprocaine, cocaine, dibucaine, dyclonine, etidocaine, isobutamben, lidocaine, mepivacaine, pramoxine, prilocalne, procaine, proparacaine, tetracaine, zolamine hydrochloride, alfentanil, chloroform, clonidine, cyclopropane, desflurane, diethyl ether, droperidol, enflurane, etomidate, fentanyl, halothane, isoflurane, ketamine hydrochloride, mepridine, methohexital, methoxyflurane, morphine, propofol, sevoflurane, sufentanil, thiamylal, thiopental, acetominophen, allopurinol, apazone, aspirin, auranofin, aurothioglucose, colchicine, diclofenac, diflunisal, etodolac, fenoprofen, flurbiprofen, gold sodium thiomalate, ibuprofen, indomethacin, ketoprofen, meclofenamate, mefenamic acid, mesalamine, methyl salicylate, nabumetone, naproxen, oxyphenbutazone, phenacetin, phenylbutazone, piroxican, salicylamide, salicylate, salicylic acid, salsalate, sulfasalazine, sulindac, tolmetin, acetophenazine, chlorpromazine, fluphenazine, mesoridazine, perphenazine, thioridazine, trifluorperazine, triflupromazine, diisopyramide, encainide, flecainide, indecanide, mexiletine, moricizine, phenytoin, procainamide, propafenone, quinidine, tocainide, cisapride, domperidone, dronabinol, haloperidol, metoclopramide, nabilone, prochlorperazine, promethazine, thiethylperazine, trimethobenzamide, buprenorphine, butorphanol, codeine, dezocine, diphenoxylate, drocode, hydrocodone, hydromorphone, levallorphan, levorphanol, loperamide, meptazinol, methadone, nalbuphine, nalmefene, nalorphine, naloxone, naltrexone, oxybutynin, oxycodone, oxymorphone, pentazocine, propoxyphene, isosorbide dinditrate, nitroglycerin, theophylline, phenylephrine, ephidrine, pilocarpine, furosemide, tetracycline, chlorpheniramine, ketorolac, ketorolac tromethamine, bromocriptine, guanabenz, prazosin, doxazosin, flufenamic acid, benzonatate, dextromethorphan hydrobromide, noscapine, codeine phosphate, scopolamine, minoxidil, combinations of the above-identified active agents, and pharmaceutically acceptable salts thereof. Other representative agents include, but are not limited to, benzodiazepines, such as alprazolan, brotizolam, chlordiazepoxide, clobazam, clonazepam, clorazepate, demoxepam, diazepam, flumazenil, flurazepan, halazepan, lorazepan, midazolam, nitrazepan, nordazepan, oxazepan, prazepam, quazepan, temazepan, triazolan, pharmaceutically acceptable salts thereof, and combinations thereof; anticholinergic agents such as anisotropine, atropine, belladonna, clidinium, cyclopentolate, dicyclomine, flavoxate, glycopyrrolate, hexocyclium, homatropine, ipratropium, isopropamide, mepenzolate, methantheline, oxyphencyclimine, pirenzepine, propantheline, telezepine, tridihexethyl, tropicamide, combinations thereof, and pharmaceutically acceptable salts thereof, estrogens, including but not limited to, 17p-estradiol (or estradiol), 17a-estradiol, chlorotrianisene, methyl estradiol, estriol, equilin, estrone, estropipate, fenestrel, mestranol, quinestrol, estrogen esters (including but not limited to estradiol cypionate, estradiol enanthate, estradiol valerate, estradiol-3-benzoate, estradiol undecylate, and estradiol 16,17-hemisuccinate), ethinyl estradiol, ethinyl estradiol-3-isopropylsulphonate, pharmaceutically acceptable salts thereof, and combinations thereof; androgens such as danazol, fluoxymesterone, methandrostenolone, methyltestosterone, nandrolone, nandrolone decanoate, nandrolone phenproprionate, oxandrolone, oxymetholone, stanozolol, testolactone, testosterone, testosterone cypionate, testosterone enanthate, testosterone propionate, 19-nortestosterone, pharmaceutically acceptable salts thereof, and combinations thereof; and progestins such as cingestol, ethynodiol diacetate, gestaclone, gestodene, bydroxyprogesterone caproate, levonorgestrel, medroxyprogesterone acetate, megestrol acetate, norgestimate, 17-deacetyl norgestimate, norethindrone, norethindrone acetate, norethynodrel, norgestrel, desogestrel, progesterone, quingestrone, tigestol, pharmaceutically acceptable salts thereof, and combinations thereof.

In some embodiments of the delivery compositions described herein, the one or more biologically active agents comprise at least 5-60% by weight of the nanofiber non-woven fabric composition. As used herein, where biologically active agents are incorporated in the nanofiber compositions described herein, dosages of the biologically active agent can be described as a % weight of the biologically active agents/quantity of fiber. For example, dosages can include 5%-60% or more by weight, such as, for example, at least 5% by weight, at least 10% by weight, at least 15% by weight, at least 20% by weight, at least 25% by weight, at least 30% by weight, at least 35% by weight, at least 40% by weight, at least 45% by weight, at least 50% by weight, at least 55% by weight, at least 60% by weight, or more. Range of dosages can include, for example, 5-10% by weight, 5-15% by weight, 5-20% by weight, 5-25% by weight, 5-30% by weight, 5-35% by weight, 5-40% by weight, 5-45% by weight, 5-50% by weight, 5-55% by weight, 5-60% by weight, 10-15% by weight, 10-20% by weight, 10-25% by weight, 10-30% by weight, 10-35% by weight, 10-40% by weight, 10-45% by weight, 10-50% by weight, 10-55% by weight, 10-60% by weight, 15-20% by weight, 15-25% by weight, 15-30% by weight, 15-35% by weight, 15-40%, 15-45% by weight, 15-50% by weight, 15-55% by weight, 15-60% by weight, 20-25% by weight, 20-30% by weight, 20-35% by weight, 20-40% by weight, 20-45% by weight, 20-50% by weight, 20-55% by weight, 20-60% by weight, 25-30% by weight, 25-35% by weight, 25-40% by weight, 25-45% by weight, 25-50% by weight, 25-55% by weight, 25-60% by weight, 30-35% by weight, 30-40% by weight, 30-45% by weight, 30-50% by weight, 30-55% by weight, 30-60% by weight, 35-40% by weight, 35-45% by weight, 35-50% by weight, 35-55% by weight, 35-60% by weight, 40-45% by weight, 40-50% by weight, 40-55% by weight, 40-60% by weight, 45-50% by weight, 45-55% by weight, 45-60% by weight, 50-55% by weight, 50-60% by weight, 55-60% by weight, of the nanofiber non-woven fabric compositions described herein.

Another key advantage of the nanofiber compositions described herein is their ability to allow for uniform distribution of one or more physicochemically diverse biologically active agents. As used herein, the term “different physicochemical properties” or “physicochemically diverse” refers to biologically active agents that fall into different categories with respect to one or more physicochemical properties. For example, two biologically active agents can have differing degrees of hydrophobicity or hydrophilicity (i.e., one is hydrophilic, and the other is hydrophobic), differing degrees of solubility (which are impacted by hydrophobicity/hydrophilicity; i.e., one is highly soluble, and the other is less Soluble—generally, a difference in solubility refers to at least one order of magnitude difference in solubility), differing partition coefficient (LogP; e.g., one has a positive LogP, the other negative—generally, a difference in partition coefficients refers to at least one order of magnitude difference in partition coefficient), differing distribution coefficient (e.g., one is positive, one is negative—generally, a difference in distribution coefficients refers to at least one order of magnitude difference in distribution coefficient), electrical charge/ionization (i.e., one is positively charged, one negatively or uncharged, or similarly, one is negatively charged, the other positively or uncharged), physical states (e.g., solid, crystalline or microcrystalline solid, particulate solid, dispersion solid, semi-solid, liquid, molecularly soluble, etc.). Other relevant properties that can differentiate one or more biologically active agents include, for example, polymeric versus monomeric form, solids suspension or particulate versus molecularly soluble, and substantially crystalline versus substantially amorphous. By “different” in this context is also meant that the biologically active agents will differ by at least 50%, by at least 75%, by at least 1-fold, by at least 2-fold, by at least 5-fold, by at least 10-fold, or more, with respect to the given property. In some embodiments, the physicochemical property is solubility in aqueous solution, and the difference is by a factor of 10-fold (i.e., an order of magnitude) or more. In general, biologically active agents that have a negative LogP are considered hydrophilic, and biologically active agents with a positive LogP are considered hydrophobic. As but one example, two biologically active agents, in which one has a negative LogP and the other has a positive LogP would be considered physicochemically diverse. However, consistent with the use of the term herein, two biologically active agents that have respective LogP values of −1 and −2 are also considered physicochemically diverse, as they differ in partition coefficient by at least an order of magnitude. For example, the USP and BP solubility classifications shown in Table 2 classify solutes, such as the biologically active agents described herein, as “very soluble” to “practically insoluble” based on the criteria shown below.

TABLE 2
USP and BP solubility criteria.

| Descriptive term | Part of solvent required per part of solute |
|---|---|
| Very soluble | Less than 1 |
| Freely soluble | From 1 to 10 |
| Soluble | From 10 to 30 |
| Sparingly soluble | From 30 to 100 |
| Slightly soluble | From 100 to 1000 |
| Very slightly soluble | From 1000 to 10,000 |
| Practically insoluble | 10,000 and over |

Typically, as used herein, a biologically active agent is considered water insoluble if it has a solubility of <0.1 mg/mL, <0.01 mg/mL, <0.001 mg/mL, or less. Similarly, a biologically active agent is considered water soluble if it has a solubility of >1 mg/mL.

The Biopharmaceutical Classification System (BCS), which groups drugs according to solubility and permeability into four different classifications, can also be used to classify biologically active agents as being physicochemically diverse, for incorporation into the nanofiber compositions described herein. The BCS classifies drugs as: Class I if they have high solubility and high permeability, Class II if they have low solubility and high permeability, Class III if they have high solubility and low permeability, and Class IV if they have low solubility and low permeability, where a drug substance is considered highly soluble when the highest dose strength is soluble in 250 ml or less of aqueous media over the pH range of 1-7.5, and a drug substance is considered to be highly permeable when the extent of absorption in humans is determined to be 90 percent or more of an administered dose.

Accordingly, in some embodiments, biologically active agents falling into Class II or Class IV of the BCS are considered water insoluble for the purposes of the nanofiber delivery compositions described herein. Non-limiting examples of BCS Class II biologically active agents include amprenavir, aripiprazole, atorvastatin, atorvastatin calcium, atovaquone, azithromycin, budesonide, calcitriol, candesartan cilexetil, carbamazepine, carisoprodol, celecoxib, clopidogrel bisulfate, clotrimazole/betamethasone, cyclosporine, dapsone, diclofenac sodium, dicyclomine hcl, dronabinol, duloxetine, dutasteride, etodolac, ezetimibe, felbamate, felodipine, fenofibrate, flecainide, fosamprenavir, furosemide, gemfibrozil, glimepiride, glipizide, glyburide, griseofulvin, hydroxychloroquine, hydroxyzine, ibuprofen, indinavir sulfate, indomethacin, irbesartan, isradipine, ketoconazole, lactulose, lamotrigine, lansoprazole, latanoprost, lopinavir/ritonavir, loracarbef, loratadine, lovastatin, mebendazole, meclizine, medroxyprogesterone acetate, meloxicam, metaxalone, methylphenidate HCl, methylphenidate HCl, methylphenidate HCl, methylprednisolone, mycophenolate mofetil, mycophenolic acid, nabumetone, naproxen, nelfinavir mesylate, nevirapine, nifedipine, olanzapine, omeprazole, oxaprozin, phenazopyridine, phenytoin sodium, pioglitazone HCl, piroxicam, primidone, prochlorperazine, pyrimethamine, quetiapine fumarate, raloxifene HCl, rifabutin, rifampin, risperidone, ritonavir, simvastatin, spironolactone, spironolactone, sulfamethoxazole, sulfasalazine, tacrolimus, tacrolimus, telmisartan, temazepam, tipranavir, travoprost, triamcinolone, ursodiol, aka ursodeoxycholic acid, valproic acid, valsartan, vardenafil, verapamil HCl, vitamin d, ergocalciferol, warfarin sodium, ziprasidone HCl and combinations thereof. Non-limiting examples of BCS Class IV biologically active agents include acetaminophen, acetazolamide, acyclovir, azathioprine, azithromycin, bisoprolol, calcitriol, carisoprodol, cefdinir, cefixime, cefuroxime axetil, cephalexin, chlorothiazide, clarithromycin, cyclosporine, dapsone, dicyclomine hcl, dronabinol, dutasteride, etoposide, furosemide, glipizide, griseofulvin, hydrochlorothiazide, indinavir sulfate, isradipine, linezolid, loperamide, mebendazole, mercaptopurine, mesalamine, methylprednisolone, modafinil, nabumetone, nelfinavir mesylate, norelgestromin, nystatin, oxcarbazepine, oxycodone HCl, progesterone, pyrimethamine, ritonavir, spironolactone, sulfamethoxazole, sulfasalazine, tadalafil, triamcinolone acetonide, trimethoprim and combinations thereof

Accordingly, in some embodiments, biologically active agents falling into Class I or Class III of the BCS are considered water soluble for the purposes of the nanofiber delivery compositions described herein. Non-limiting examples of BCS class I biologically active agents include those listed in Kasim et al. Mol. Pharmaceutics 1(1): 85-96 (2004) and Lindenberger et al. Eur. J. Pharm. Biopharm. 58(2):265-78 (2004), the contents of which are herein incorporated by reference in their entireties, such as amitriptyline hydrochloride, biperiden hydrochloride, chloroquine phosphate, chlorpheniramine maleate, chlorpromazine hydrochloride, clomiphene citrate, cloxacillin sodium, ergotamine tartrate, indinavir sulfate, levamisole hydrochloride, levothyroxine sodium, mefloquine hydrochloride, nelfinavir mesylate, neostigmine bromide, phenytoin sodium, prednisolone, promethazine hydrochloride, proguanil hydrochloride, quinine sulfate, salbutamol, warfarin sodium, caffeine, fluvastatin, Metoprolol tartrate, Propranolol, theophylline, verapamil, Diltiazem, Gabapentin, Levodopa, carbidopa, reserpine, ethynyl estradiol, norethindrone, saquinavir mesylate and Divalproex sodium. Non-limiting examples of BCS class III biologically active agents include proteins, peptides, polysaccharides, nucleic acids, nucleic acid oligomers and viruses, and abacavir sulfate, amiloride HCl, atropine sulfate, chloramphenicol, folic acid, hydrochlorthazide, lamivudine, methyldopa, mefloquine HCl, penicillamine, pyrazinamide, salbutamol sulfate, valproic acid, stavudine, ethosuximide, ergometrine maleate, colchicines, didanosine, cimetidine, ciprofloxacin, neomycin B, captopril, Atenolol, and Caspofungin.

Accordingly, in some embodiments of the delivery compositions described herein, the one or more physicochemically diverse biologically active agents are selected from tenofovir (water soluble >1 mg/mL), dapivirine (water insoluble <0.001 mg/mL), levonorgestrel (water insoluble <0.01 mg/mL), etravirine (water insoluble <0.1 mg/mL), raltegravir (ionizable acidic drug and also Potassium salt, pKa 7), and maraviroc (ionizable basic drug, pKa 8).

In some embodiments of the delivery compositions described herein, the one or more physicochemically diverse biologically active agents are electrospun in different solid states. For example, where one biologically active agent is electrospun as a crystalline solid dispersion and the other biologically active agent is molecularly dispersed.

In some embodiments of the delivery compositions described herein, the two or more biologically active agents are selected from tenofovir, dapivirine, levonorgestrel, etravirine, raltegravir, and maraviroc.

Also provided herein, in some aspects, are composite biologically active agent-delivery compositions comprising one or more layers. As demonstrated herein, depending on the different physicochemical properties of two or more biologically active agents, they can distribute within the uniform nanofiber compositions differently, such that compositions comprising two or more different layers can be used to allow for uniform distribution of the different biologically active agents in a single product. For example, it is shown herein that, when highly water insoluble biologically active agents are used, they show uniform distribution and thus can be electrospun into a nanofiber delivery composition together. However, if the different biologically active agents are both water soluble, and ionizable, but are basic in nature, different layers can be required to allow for uniform distribution of the different biologically active agents. Thus, the physicochemical properties of two or more biologically active agents can determine the type of composite biologically active agent-delivery compositions needed.

Accordingly, the composite biologically active agent-delivery compositions comprising one or more layers can, in some aspects, comprise one layer of two or more biologically active agents. In other aspects, composite biologically active agent-delivery compositions can comprise individually electrospun layers, each of which comprises one or more biologically active agents, such that the individually formed layers are combined with each another. In other aspects, composite biologically active agent-delivery compositions can comprise a first electrospun layer comprising one or more biologically active agents, and two or more additional layers, each of which are directly electrospun upon the previous one or more layers, and each of which layers comprises one or more biologically active agents. In some embodiments of these aspects and all such aspects described, the polymer used in different layers of the composite biologically active agent-delivery compositions is the same in two or more layers. In some embodiments of these aspects and all such aspects described, the polymer used in different layers of the composite biologically active agent-delivery compositions is different in two or more layers.

Thus, in some aspects, provided herein are composite biologically active agent-delivery compositions comprising a first layer of uniform high basis weight, non-woven, polymer nanofiber fabric composition comprising a first biologically active agent and a second layer of uniform high basis weight, non-woven, polymer nanofiber fabric composition comprising a second biologically active agent, such that the polymer is the same in the first and second layers.

In some embodiments of such composite biologically active agent-delivery compositions, each of the nanofiber non-woven fabric compositions is uniform over an area of at least at least 25 cm (e.g., in the CD dimension) by at least 100 cm (e.g., in the MD dimension).

In some embodiments of the composite biologically active agent-delivery composition, the weight of any 1 cm disc obtained from the area of at least 25 cm (e.g., in the CD dimension) by at least 100 cm (e.g., in the MD dimension) is within 10% of the mean basis weight over the entire area of at least 25 cm by at least 100 cm.

In some embodiments of the composite biologically active agent-delivery composition the basis weight is in the range of 50-500 gm/m², inclusive.

In some embodiments of the composite biologically active agent-delivery composition, at least one of the nanofiber non-woven fabric compositions is produced by an electrospinning method. In some embodiments, the electrospinning is performed using a nozzle-less electrospinning method.

In some embodiments of the composite biologically active agent-delivery composition, the polymer is rapidly water soluble. Such a rapidly water soluble polymer provides burst biologically active agent release of the first and second biologically active agents. In some embodiments, the rapidly water soluble polymer is selected from polyvinyl alcohol, polyethylene oxide, polyvinylpyrrolidone, poly-2-ethyl-2-oxazoline, and polyacrylic acid, among others.

In other embodiments, the composite biologically active agent-delivery composition, the polymer provides sustained biologically active agent release. In such embodiments, the polymer can be selected from, e.g., poly[lactic-co-glycolic] acid, polycaprolactone, and ethyl cellulose, among others.

In certain embodiments of a composite biologically active agent-delivery composition, the first and second biologically active agents are selected from tenofovir, dapivirine, levonorgestrel, etravirine, raltegravir, and maraviroc.

In another aspect, provided herein are composite biologically active agent-delivery compositions comprising a first layer of uniform high basis weight, non-woven, polymer nanofiber fabric composition comprising a first biologically active agent and a second layer of uniform high basis weight, non-woven, polymer nanofiber fabric composition comprising a second biologically active agent, where each of the layers are separately produced and then combined into the composite compositions. In some embodiments of these composite biologically active agent-delivery compositions, the polymer can be different in the first and second layers. In some embodiments of such a composite biologically active agent-delivery composition, each of the nanofiber non-woven fabric compositions is uniform over an area of at least 25 cm (e.g., in the CD dimension) by at least 100 cm (e.g., in the MD dimension).

In some embodiments of such a composite biologically active agent-delivery composition, the weight of any 1 cm disc obtained from the area of at least 25 cm (e.g., in the CD dimension) by at least 100 cm (e.g., in the MD dimension) is within 10% of the mean basis weight over the entire area of at least 25 cm by at least 100 cm.

In some embodiments of such a composite, the basis weight of each layer is in the range of 50-500 gm/m², inclusive.

In some embodiments of such a composite, at least one of the nanofiber non-woven fabric compositions is produced by an electrospinning method. The electrospinning method can be a nozzle-less electrospinning method.

In some embodiments of such a composite, either or both of the different polymers is/are rapidly water soluble. As noted above, a rapidly water soluble polymer provides burst biologically active agent release of the first and second biologically active agents. Rapidly water soluble polymer can be selected, for example, from polyvinyl alcohol, polyethylene oxide, polyvinylpyrrolidone, poly-2-ethyl-2-oxazoline, and polyacrylic acid, among others.

In some embodiments of such a composite, either or both of the polymers provide(s) sustained biologically active agent release. As noted above, polymers selected from poly[lactic-co-glycolic] acid, polycaprolactone, and ethyl cellulose, among others, can provide sustained release characteristics.

In some embodiments, the first layer polymer is rapidly water soluble, such that the first layer provides burst release kinetics, and the second layer polymer provides sustained biologically active agent release. Rapidly water soluble polymers useful for such layers are described above or known in the art. Similarly, polymers that provide sustained release kinetics are described above or known in the art.

In certain embodiments, such a composite biologically active agent-delivery composition can include first and second biologically active agents selected from tenofovir, dapivirine, levonorgestrel, etravirine, raltegravir, and maraviroc, among others.

In another aspect, provided herein are composite biologically active agent-delivery compositions comprising a first layer of uniform high basis weight, non-woven, polymer nanofiber fabric composition comprising a first biologically active agent and a second layer of uniform high basis weight, non-woven, polymer nanofiber fabric composition comprising a second biologically active agent, where the second layer of uniform high basis weight, non-woven, polymer nanofiber fabric composition comprising a second biologically active agent is directly electrospun onto the first layer. In some embodiments of these composite biologically active agent-delivery compositions, the polymer can be different in the first and second layers. In some embodiments of these composite biologically active agent-delivery compositions, the polymer is the same in the first and second layers. In some embodiments of such a composite biologically active agent-delivery composition, each of the nanofiber non-woven fabric compositions is uniform over an area of at least 25 cm (e.g., in the CD dimension) by at least 100 cm (e.g., in the MD dimension).

In some embodiments of such a composite biologically active agent-delivery composition, the weight of any 1 cm disc obtained from the area of at least 25 cm (e.g., in the CD dimension) by at least 100 cm (e.g., in the MD dimension) is within 10% of the mean basis weight over the entire area of at least at least 25 cm by at least 100 cm.

In some embodiments of such a composite, the basis weight of each layer is in the range of 50-500 gm/m², inclusive.

In some embodiments of such a composite, at least one of the nanofiber non-woven fabric compositions is produced by an electrospinning method. The electrospinning method can be a nozzle-less electrospinning method.

In some embodiments of such a composite, either or both of the different polymers is/are rapidly water soluble. As noted above, a rapidly water soluble polymer provides burst biologically active agent release of the first and second biologically active agents. Rapidly water soluble polymer can be selected, for example, from polyvinyl alcohol, polyethylene oxide, polyvinylpyrrolidone, poly-2-ethyl-2-oxazoline, and polyacrylic acid, among others.

In some embodiments of such a composite, either or both of the polymers provide(s) sustained biologically active agent release. As noted above, polymers selected from poly[lactic-co-glycolic] acid, polycaprolactone, and ethyl cellulose, among others, can provide sustained release characteristics.

In some embodiments, the first layer polymer is rapidly water soluble, such that the first layer provides burst release kinetics, and the second layer polymer provides sustained biologically active agent release. Rapidly water soluble polymers useful for such layers are described above or known in the art. Similarly, polymers that provide sustained release kinetics are described above or known in the art.

In certain embodiments, such a composite biologically active agent-delivery composition can include first and second biologically active agents selected from tenofovir, dapivirine, levonorgestrel, etravirine, raltegravir, and maraviroc, among others.

Nanofiber non-woven fabric compositions described herein can be fabricated by a method comprising electrospinning fibers from a solution comprising a polymer dissolved in a solvent. As discussed above, the electrospinning method can include a nozzle-less, needle-less or so called free surface electrospinning method—such methods permit a higher degree of uniformity in the resulting non-woven, nanofiber polymer fabric, over a greater area than nozzle- or needle-fed electrospinning, and the area of uniformity, at high basis weight, can be increased relative to standard nozzle-less or needle-less electrospinning using the modifications described herein above. Indeed, the device described herein in which a nozzle-less or needle-less electrospinning device is modified with one or more of collecting electrode insulation, shielding, collimating shielding, or additional electrode connections for the electrospinning electrode and/or collecting electrode can produce nanospun fabrics at basis weight and uniformity not achievable with existing electrospinning devices. Thus, a uniform, high basis weight, non-woven polymer nanofiber fabric composition produced by a device including any combination or all of the modifications described herein will necessarily differ structurally from a non-woven polymer nanofiber fabric composition produced using an existing device.

Also provided herein, in some aspects, are optimization methods and modules to determine the values of the parameters that should be used to obtain the desired throughput, content uniformity, and material yield of a uniform, high basis weight, non-woven polymer nanofiber fabric composition using any of the devices described herein, as well as, in some aspects, any of the electrospinning devices known in the art. The optimization methods, modules, and processes described herein can be used separately or in conjunction with the devices and improvements described herein and provide insight into how the electrospinning process itself works and identifies methods by which electrospinning can be controlled to be highly productive and significantly increase the uniformity of a high basis weight, non-woven polymer nanofiber fabric composition described herein.

In some aspects, provided herein are functional optimization modules that can be used to optimize throughput and uniformity for a given polymer solution depending on the settings of a free-surface electrospinning device, such as a NANOSPIDER™. The optimization provides for the identification of optimized polymer solution parameters and machine settings for a particular polymer solution. For example, throughput can be measured empirically after a series of experiments and quantified into a modular function. To identify optimal parameters for uniformity, however, other outcomes are measured and a functional module is created into which those outcomes are fed such that optimal parameters are identified and provided as an output.

In some aspects, the optimization methods comprise first collecting data by conducting a static first run with the polymer material for which optimization is required. Input data is collected from the static run along with, for example, high quality video of the spinning electrode wire during electrospinning Such input data includes, for example, machine direction distribution profile, cross direction single jet standard deviation, jetting time, jet initiation time, jet-time profile, jet spacing, and entrainment volume. Subsequently, the measured quantities from this static first run are provided as inputs into the empirical modules provided herein to describe the mass deposition profile of the polymer material. The model can then be used to determine how to alter the solution and processing parameters to achieve desired or optimized throughput, content uniformity, and material yield, in some embodiments.

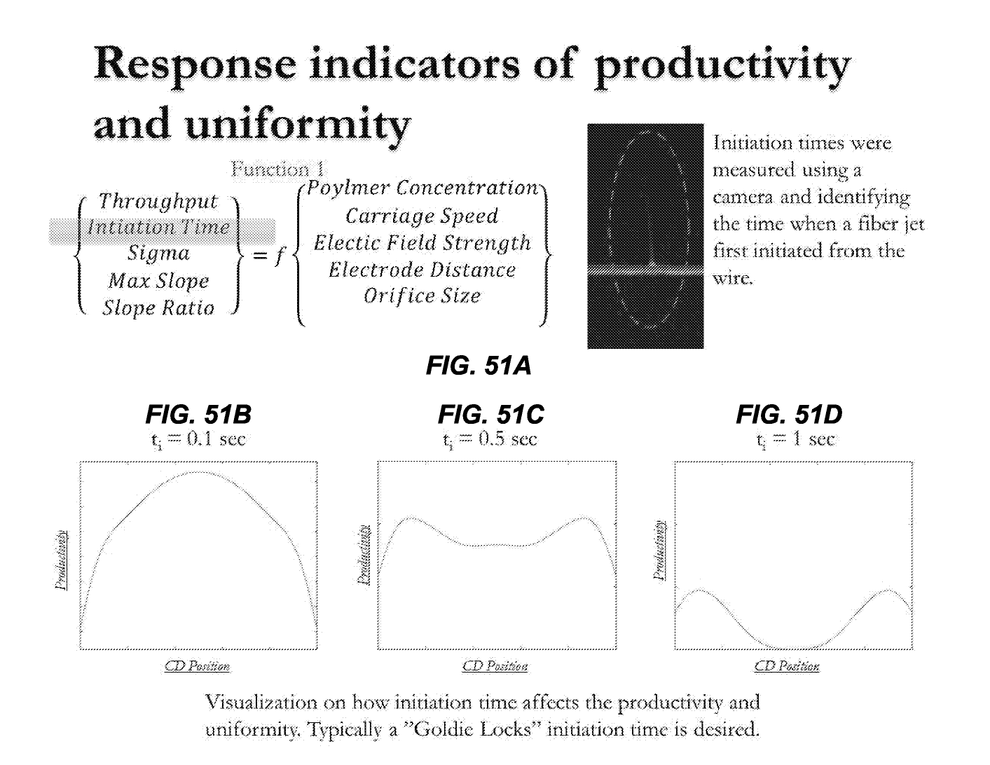

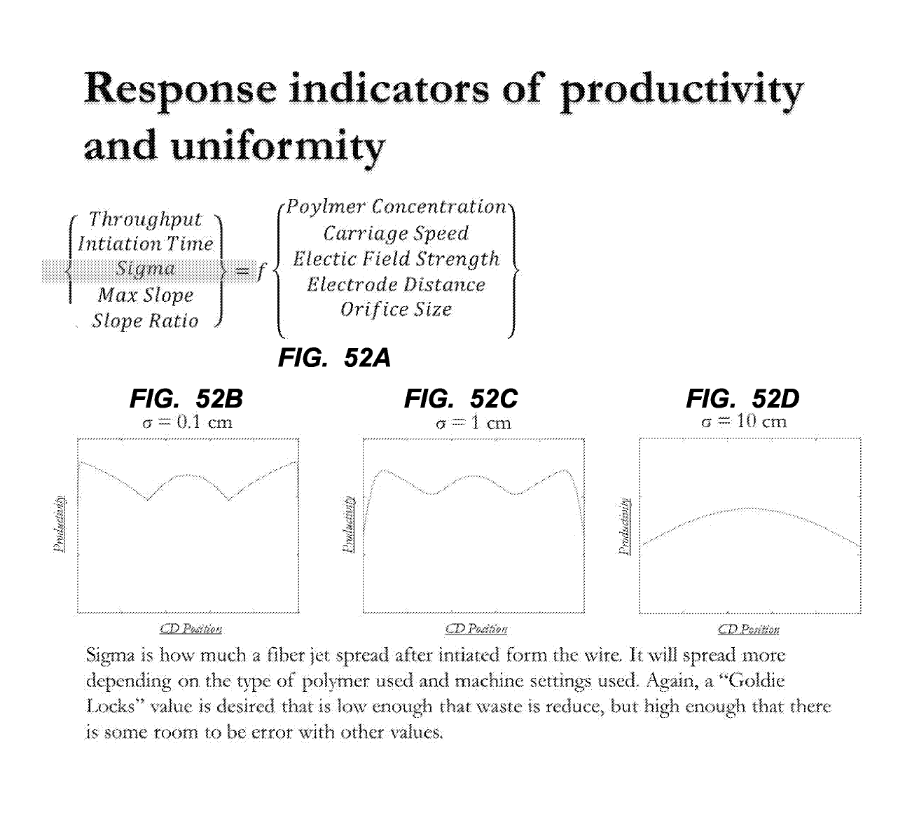

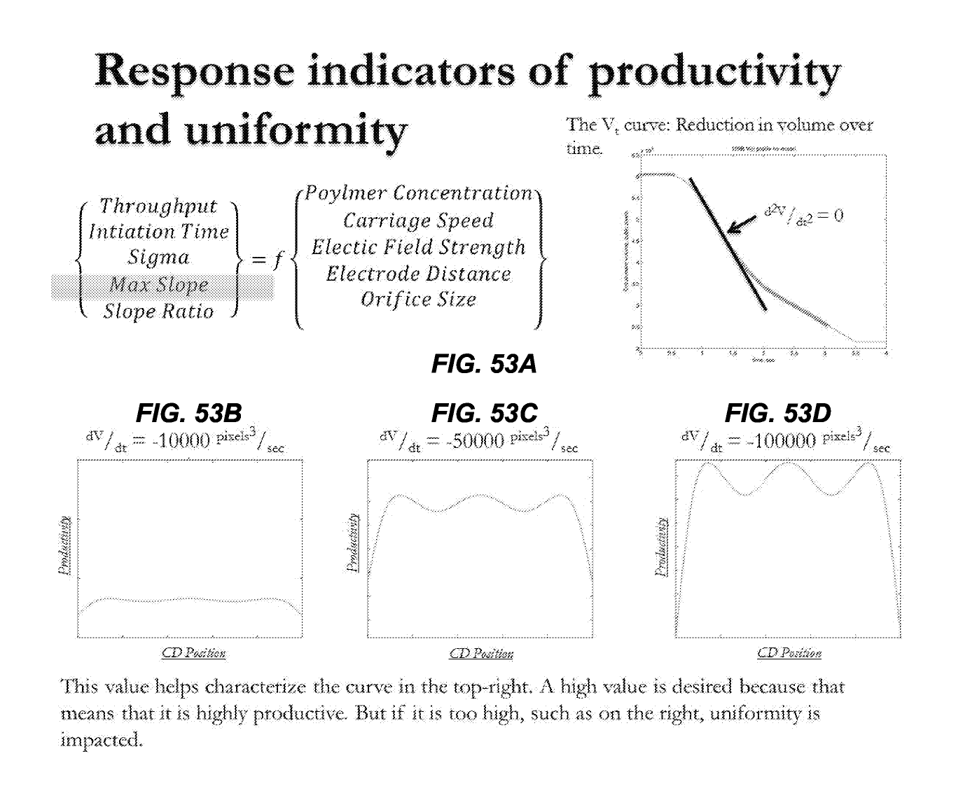

For example, as shown herein at FIGS. 51-53, six variables, including drug concentration, were varied on the right hand side of a functional module using an experiment designed to determine how the six variables changed the outcomes on the left hand side of the functional module (function 1). The outcomes, including throughput, initiation time, sigma, maximum slope and slope ratio, were outcomes that were measured and fed into a model to predict uniformity. The functional modules described herein provide quantifiable methods that can be used to determine how the volume that is entrained on the wire can change for different variable (values shown on the right hand side of the functional module) levels.

As used herein, the variable “initiation time” refers to the time it takes for a polymer jet to initiate after the carriage of a device has passed over a spot. As shown herein, higher surface tensions of a polymer solution appear to initiate less reliably in the same place. As also demonstrated herein, viscosity and electric field strength can also impact initiation time, as can carriage speed of a device, if the polymer solution is non-Newtonian When keeping all other variables equal, it is important to note that increasing the initiation time will decrease the throughput of the usable area of the high basis weight, non-woven polymer nanofiber fabric composition produced from the polymer solution.

As used herein, the variable “sigma” refers to how much a polymer fiber jet spreads after initiating from the wire of the electrospinning electrode. A polymer fiber jet's spread varies depending on the type of polymer used and machine settings used. Again, an optimal sigma value is desired that is low enough that waste is reduced, but high enough that there is some room for variability in conjunction with other values. Without wishing to be bound or limited by theory, it is assumed that the spread of a fiber jet has a Gaussian distribution of mass.

As used herein, the variable “max slope” or “maximum slope” refers to the point at which the polymer is spinning most intensely. Max slope affects throughput of a high basis weight, non-woven polymer nanofiber fabric composition, but increasing the magnitude of the value of max slope will also amplify whatever features are present in the profile and therefore impact uniformity.

As used herein, the variable “slope ratio” refers to a qualitative measure of how intensely a polymer solution is spinning from the wire, such that R approaches 0 indicates High intensity spinning, R approaches 1 indicates Low intensity spinning Depending on the value of “R,” the mass deposition of the polymer high basis weight, non-woven polymer nanofiber fabric composition will be focused either in the center, edges, or neither.

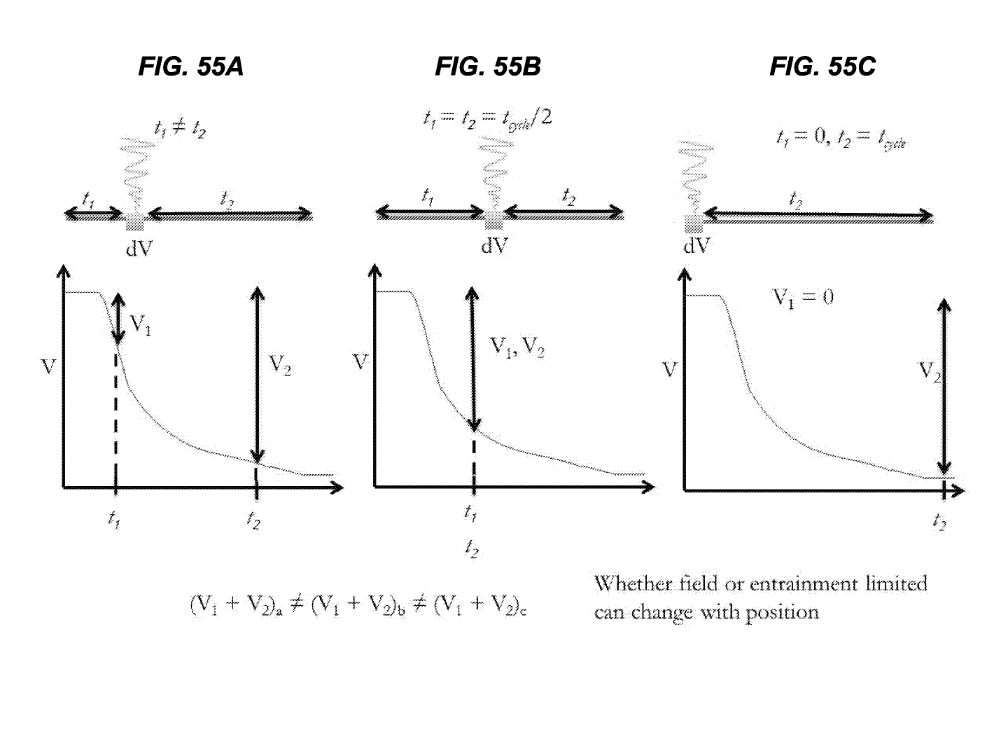

Accordingly, using the quantified outcomes from the optimization modules established herein between the outcomes and variables, the change in volume over time curve can be used to determine how the polymer fibers will be deposited on a substrate given different variables (e.g., polymer concentration, carriage speed, etc.) levels, as shown at FIG. 55. Different carriage speeds indicate that different amounts of polymer fiber get deposited on the substrate since the rate at which the volume is reduced on the wire is not linear.

Once a V(t) curve is obtained, the mass that is deposited on the substrate can calculated by determining the difference between the initial entrainment and any point on the curve, for every point on the wire. Using a predicted curve of volume reduction over time, a mass distribution curve can be constructed using the optimization modules described herein. By constructing several of these distributions, an optimal distribution (greatest uniformity, highest throughput) can be determined using the optimization modules described herein.

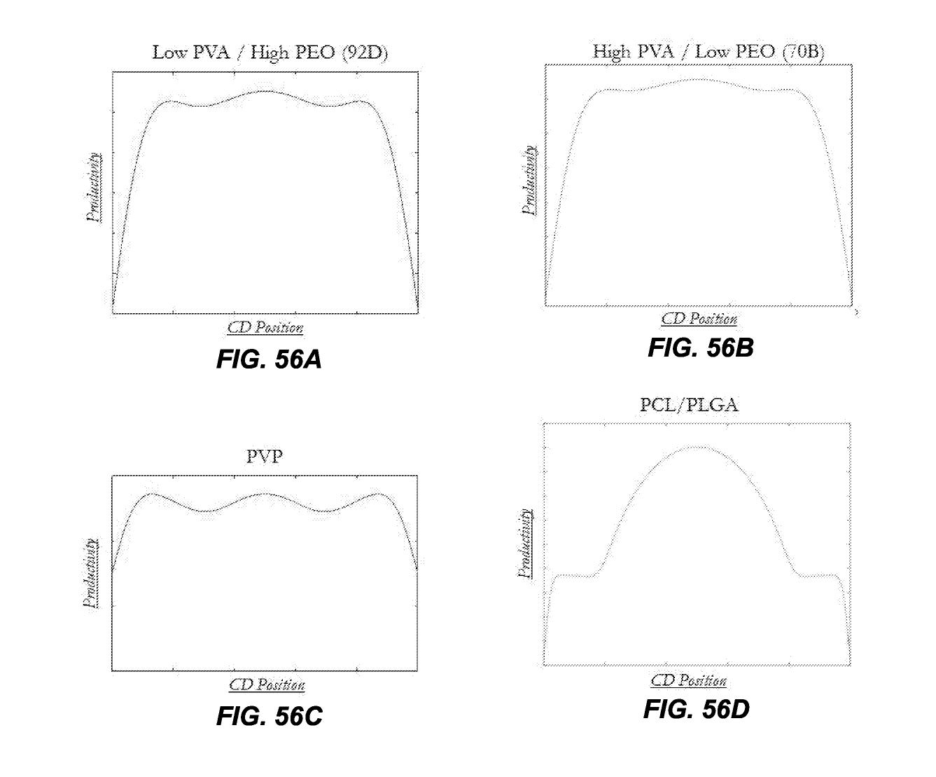

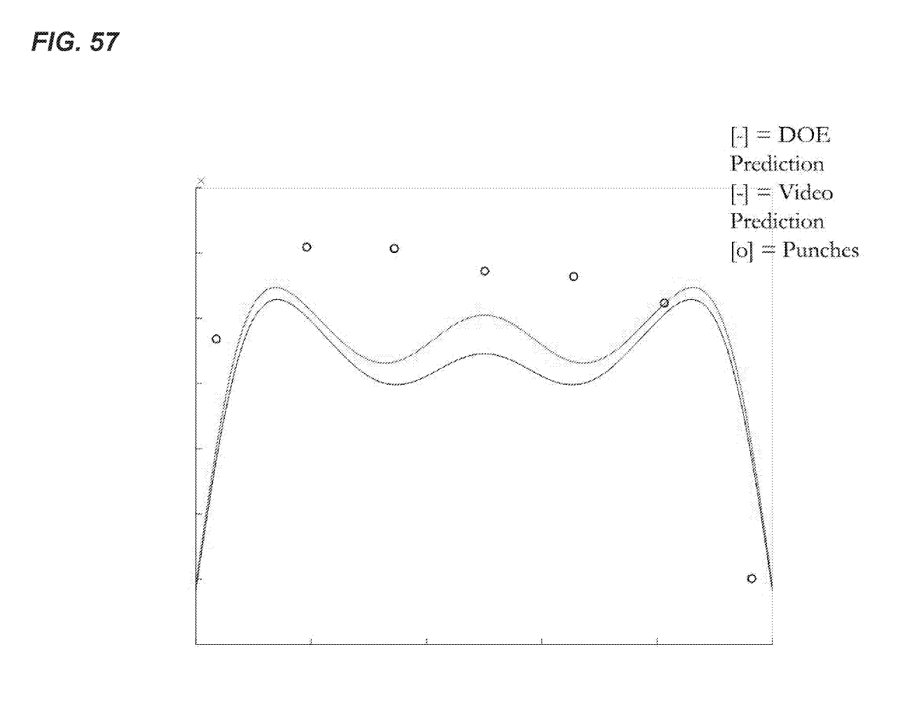

For example, optimal distributions for four different formulations are shown herein at FIG. 56. To confirm the accuracy of the optimization modules described herein, the parameters that are predicted to create an optimal distribution are used in practice and the empirical data are compared to the prediction, as shown in FIG. 57. “Video prediction” refers to the prediction based upon data gained from a camera used to directly observe the reduction in volume over time for a specific run of a polymer solution in a device. “Punches” refers to the empirical mass data gathered to compare to the two predictions. As shown at FIG. 56, for each formulation, uniformity typically plateaued at ˜30-35% of the carriage direction length. The percentage is the uniform Carriage Direction length over the total Carriage Direction fiber length. In regards to optimal productivities (gm/hour), non-limiting examples for various polymer formulations include, 70B: 12 grams/hour; 92D: 6 grams/hour; PVP: 4 grams/hour; and PLGA/PCL: 35 grams/hour.

It is understood that the foregoing description and examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

We claim:

1. A nozzle-less electrospinning device comprising:
an electrospinning electrode and a collecting electrode comprising connections for a DC power supply, the electrospinning electrode and the collecting electrode spaced apart and establishing an electric field between the electrospinning electrode and the collecting electrode when DC power is supplied;
the collecting electrode comprising a first end and a second end;
the electrospinning electrode comprising a continuously fed or static charged electrode member partially submerged or carrying an entrained polymer solution to permit electrospinning fibers of the polymer towards the collecting electrode;
a substrate located between the electrospinning electrode and the collecting electrode such that electrospun polymer fibers become deposited on the substantially planar substrate when in use;
a first shield comprised of a first insulating material member, having a dielectric constant of at least 1.2, situated between the substrate and the collecting electrode and extending from the first end of the collecting electrode towards the second end of the collecting electrode such that a portion of the electric field is shielded by the first insulating material member and a gap of unshielded collecting electrode is formed extending from an end of the first insulating material member towards the second end of the collecting electrode;
wherein the first shield increases the uniform area of an electrospun polymer mat deposited on the substrate relative to the uniform area of a polymer mat deposited in the absence of the first shield.

2. The nozzle-less electrospinning device of claim 1, further comprising a second shield comprised of a second insulating material member, having a dielectric constant of at least 1.2, situated between the substrate and the collecting electrode and extending from the second end of the collecting electrode towards the first end of the collecting electrode such that a portion of the electric field is shielded by the second insulating material member and the gap of unshielded collecting electrode extends between an end of the first insulating material member and an end of the second insulating material member, wherein the second shield further increases the uniform area of an electrospun polymer mat deposited on the substrate relative to the uniform area of a polymer mat deposited in the absence of the second shield.

3. The nozzle-less electrospinning device of claim 1, further comprising a first collimating shield comprised of a third insulating material member, having a dielectric constant of at least 1.2, the first collimating shield supported and situated adjacent to the substrate and between the substrate and the electrospinning electrode, the first collimating shield extending substantially perpendicular to the collecting electrode, an edge of the first collimating shield facing the gap of unshielded collecting electrode aligned with the end of the first shield insulating material member adjacent the gap of unshielded collecting electrode, wherein the first collimating shield increases the uniform area of an electrospun polymer mat deposited on the substrate relative to the uniform area of a polymer mat deposited in the absence of the collimating shield.

4. The nozzle-less electrospinning device of claim 3, further comprising a second collimating shield comprised of a fourth insulating material member, having a dielectric constant of at least 1.2, the second collimating shield supported and situated adjacent to the substrate and between the substrate and the electrospinning electrode, the second collimating shield extending substantially perpendicular to the collecting electrode, an edge of the second collimating shield facing the gap of unshielded collecting electrode aligned with the end of the first shield insulating material member adjacent the gap of unshielded collecting electrode, wherein the first collimating shield increases the uniform area of an electrospun polymer mat deposited on the substrate relative to the uniform area of a polymer mat deposited in the absence of the collimating shield.

5. The nozzle-less electrospinning device of claim 4, further comprising a first encircling insulating material member, having a dielectric constant of at least 1.2, encircling the collecting electrode and extending along the collecting electrode from the first end of the collecting electrode towards the second end of the collecting electrode such that a portion of the electrode is covered by the first encircling insulating material member and a gap of exposed collecting electrode is formed extending from an end of the first encircling insulating material member towards the second end of the collecting electrode.

6. The nozzle-less electrospinning device of claim 5, further comprising a second encircling insulating material member, having a dielectric constant of at least 1.2, encircling the collecting electrode and extending along the collecting electrode from the second end of the collecting electrode towards the first end of the collecting electrode such that a portion of the electrode is covered by the second encircling insulating material member and a gap of exposed collecting electrode is defined extending from an end of the first encircling insulating material member to an end of the second encircling insulating material member.

7. The nozzle-less electrospinning device of claim 1, wherein the uniform area of the electrospun polymer mat deposited on the substrate by the device in the presence of the first insulating material member is at least 25 cm by 100 cm.

8. The nozzle-less electrospinning device of claim 1, wherein the substrate is substantially planar.

9. The nozzle-less electrospinning device of claim 1, wherein the substrate is configured to move perpendicular to the direction of the collecting electrode when the device is in use.

10. The nozzle-less electrospinning device of claim 1, wherein the electrospinning electrode comprises a charged surface from which fibers are electrospun, and wherein the length of the gap of exposed collecting electrode is aligned with and substantially the same length as the charged surface of the electrospinning electrode from which fibers are electrospun.

11. A uniform high basis weight, non-woven, polymer nanofiber fabric composition, wherein the composition is uniform over an area of at least 25 cm by 100 cm.

12. The composition of claim 11, wherein the weight of any 1 cm disc obtained from the area of at least 25 cm by 100 cm is within 10% of a mean basis weight over the entire area of at least 25 cm by 100 cm.

13. The composition of claim 11, having a basis weight is in the range of 50-500 gm/m², inclusive.

14. The composition of claim 11, wherein the nanofiber non-woven fabric composition is produced by an electrospinning method.

15. A biologically active agent-delivery composition comprising the nanofiber non-woven fabric composition of claim 11, wherein the fabric comprises a uniform distribution of one or more biologically active agents.

16. The biologically active agent-delivery composition of claim 15, wherein the nanofiber non-woven fabric composition comprises at least 5-60% by weight of the one or more biologically active agents.

17. The biologically active agent-delivery composition of claim 15, wherein the one or more biologically active agents are selected from tenofovir, dapivirine, levonorgestrel, etravirine, raltegravir, and maraviroc.

18. A composite biologically active agent-delivery composition comprising a first layer of uniform high basis weight, non-woven, polymer nanofiber fabric composition comprising a first biologically active agent, and a second layer of uniform high basis weight, non-woven, polymer nanofiber fabric composition comprising a second biologically active agent, wherein each of the nanofiber non-woven fabric compositions is uniform over an area of at least 25 cm by 100 cm and wherein each of the nanofiber non-woven fabric compositions has a basis weight in the range of 50-500 gm/m², inclusive.

19. The composite biologically active agent-delivery composition of claim 18, wherein the polymer is different in the first and second layers, and wherein one layer provides sustained biologically active agent release and the other layer provides burst biologically active agent release.

20. A method of producing the biologically active agent-delivery composition of claim 15, the method comprising electrospinning fibers from a solution comprising a polymer and one or more biologically active agents from a nozzle-less electrospinning device.
